(19) United States
(12) Patent Application Publication
(10) Pub. No.: US 2026/0245734 A1
(43) Pub. Date: Aug. 20, 2026

(54) METHODS AND SYSTEMS FOR DETERMINING A PREGNANCY-RELATED STATE OF A SUBJECT

(71) Applicant: Mirvie, Inc., South San Francisco, CA (US)

(72) Inventors: Maneesh JAIN, San Francisco, CA (US); Eugeni NAMSARAEV, Santa Clara, CA (US); Morten RASMUSSEN, South San Francisco, CA (US); Joan Camunas SOLER, South San Francisco, CA (US); Farooq SIDDIQUI, South San Francisco, CA (US); Mitsu REDDY, South San Francisco, CA (US); Elaine GEE, South San Francisco, CA (US); Arkady KHODURSKY, South San Francisco, CA (US); Rory NOLAN, South San Francisco, CA (US); Manfred LEE, Belmont, CA (US); Alison Anne Dormer COWAN, South San Francisco, CA (US); Nathaniel DELANEY-BUSCH, South San Francisco, CA (US); Aram GIAHI SARAVANI, South San Francisco, CA (US)

(21) Appl. No.: 19/374,611

(22) Filed: Oct. 30, 2025

Related U.S. Application Data
(63) Continuation of application No. PCT/US2024/027444, filed on May 2, 2024.
(60) Provisional application No. 63/463,781, filed on May 3, 2023.
(60) Provisional application No. 63/631,100, filed on Apr. 8, 2024.

(51) Int. Cl. G16H 50/30; C12Q 1/6809; C12Q 1/6883; G16H 20/10; G16H 50/70

(52) U.S. Cl. CPC G16H 50/30; C12Q 1/6809; C12Q 1/6883; G16H 20/10; G16H 50/70; C12Q 2600/158

(57) ABSTRACT

The present disclosure provides methods and systems directed to cell-free identification and/or monitoring of pregnancy-related states. A method for identifying or monitoring a presence or elevated risk of a pregnancy-related state of a pregnant subject may comprise assaying a cell-free biological sample derived from said pregnant subject to detect a set of biomarkers, and analyzing the set of biomarkers with a trained algorithm to determine the presence or elevated risk of the pregnancy-related state.

### CROSS-REFERENCE

This application is a continuation of International Application No. PCT/US2024/027444, filed May 2, 2024, which claims the benefit of U.S. Provisional Application No. 63/463,781, filed May 3, 2023, and U.S. Provisional Application No. 63/631,100, filed Apr. 8, 2024, each of which is incorporated by reference herein in its entirety.

### BACKGROUND

Every year, about 15 million pre-term births as well as 10 million cases of preeclampsia are reported globally, and over 300,000 women die of pregnancy related complications such as hemorrhage and hypertensive disorders like preeclampsia. Pre-term birth may affect as many as about 10% of pregnancies, of which the majority are spontaneous pre-term births. Preeclampsia may affect as many as about 8% of pregnancies, and is a leading cause of maternal mortality and morbidity. Pregnancy-related complications such as pre-term birth are a leading cause of neonatal death and of complications later in life. Further, such pregnancy-related complications can cause negative health effects on maternal health.

### SUMMARY

Currently, there may be a lack of meaningful, clinically actionable diagnostic screenings or tests available for many pregnancy-related complications such as pre-term birth (e.g., spontaneous pre-term birth or medically-indicated pre-term birth), preeclampsia, and growth restriction of the fetus. Antenatal screening tools may fail to detect up to 70% of pregnancies with a growth restricted fetus, therefore failing to identify those at greatest risk for adverse outcomes, including fetal mortality.

Thus, to make pregnancy as safe as possible, there exists a need for rapid, accurate methods for identifying and monitoring pregnancy-related states that are non-invasive and cost-effective, toward improving maternal and fetal health.

The present disclosure provides methods, systems, and kits for identifying or monitoring pregnancy-related states by processing cell-free biological samples obtained from or derived from subjects. Cell-free biological samples (e.g., plasma samples) obtained from subjects may be analyzed to identify the pregnancy-related state (which may include, e.g., measuring a presence, absence, or relative assessment of the pregnancy-related state). Such subjects may include subjects with one or more pregnancy-related states and subjects without pregnancy-related states. Pregnancy-related states may include, for example, pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In an aspect, the present disclosure provides a method for identifying a presence or elevated risk of a pregnancy-related state of a pregnant subject, comprising assaying a cell-free biological sample derived from the pregnant subject to detect a set of biomarkers, and processing the set of biomarkers with a trained algorithm or against a reference value to determine the presence or elevated risk of the pregnancy-related state among a set of at least three distinct pregnancy-related states.

In some embodiments, the pregnancy-related state is selected from the group consisting of pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development).

In some embodiments, the pregnancy-related state is a subtype of pre-term birth, and the at least three distinct pregnancy-related states include at least two distinct subtypes of pre-term birth. In some embodiments, the subtype of pre-term birth is a molecular subtype of pre-term birth, and the at least two distinct subtypes of pre-term birth include at least two distinct molecular subtypes of pre-term birth. In some embodiments, the molecular subtype of pre-term birth is selected from the group consisting of history of prior pre-term birth, spontaneous pre-term birth, race/ethnicity specific pre-term birth risk, and preterm prelabor rupture of membranes (PPROM). In some embodiments, the molecular subtype of pre-term birth is spontaneous pre-term birth, and the set of biomarkers comprises a genomic locus associated with spontaneous pre-term birth. In some embodiments, the genomic locus associated with spontaneous pre-term birth is selected from the group consisting of genes listed in Table 1, genes listed in Table 2, genes listed in Table 3, genes corresponding to a pathway listed in Table 4, genes listed in Table 9, genes listed in Table 10, and genes listed in Table 11. In some embodiments, the spontaneous pre-term birth comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the method further comprises identifying a clinical intervention for the pregnant subject based at least in part on the presence or elevated risk of the molecular subtype of pre-term birth. In some embodiments, the clinical intervention is selected from a plurality of clinical interventions. In some embodiments, the clinical intervention comprises a drug, a supplement, a lifestyle recommendation, a cervical cerclage, a cervical pessary, electrical contraction inhibition, ultrasound surveillance, serial cervical length assessment, or diagnostic testing (e.g., fetal fibronectin (fFN) test or infectious testing). In some embodiments, the drug is selected from the group consisting of progesterone, erythromycin, azithromycin, a tocolytic medication, a corticosteroid, a vaginal flora, and an antioxidant.

In some embodiments, the pregnancy-related state is a subtype of preeclampsia and the at least three distinct pregnancy-related states include at least two distinct subtypes of preeclampsia, at least three distinct subtypes of preeclampsia, or at least four distinct subtypes of preeclampsia. In some embodiments, the subtype of preeclampsia is a molecular subtype of preeclampsia, and the at least two distinct subtypes of preeclampsia include at least two distinct molecular subtypes of preeclampsia, at least three distinct subtypes of preeclampsia, or at least four distinct subtypes of preeclampsia. In some embodiments, the molecular subtype of preeclampsia is selected from the group consisting of history of chronic or pre-existing hypertension, superimposed preeclampsia, presence or history of gestational hypertension, presence or history of preeclampsia without severe features, presence or history of preeclampsia with severe features, presence or history of eclampsia, presence or history of preeclampsia with severe features, and presence or history of hemolysis, elevated liver enzymes, and low platelets (HELLP) syndrome. In some embodiments, the molecular subtype of preeclampsia is pre-term preeclampsia or term preeclampsia with severe features, and the set of biomarkers comprises a genomic locus associated with pre-term preeclampsia or term preeclampsia with severe features. In some embodiments, the genomic locus associated with pre-term preeclampsia or term preeclampsia with severe features is selected from the group consisting of genes listed in Table 5, genes listed in Table 6, genes listed in Table 7, genes listed in Table 8, genes listed in Table 21, genes listed in Table 22, genes listed in Table 23, and genes listed in Table 24.

In some embodiments, the pre-term preeclampsia comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the method further comprises identifying a clinical intervention for the pregnant subject based at least in part on the presence or elevated risk of the molecular subtype of preeclampsia. In some embodiments, the clinical intervention is selected from a plurality of clinical interventions. In some embodiments, the clinical intervention comprises include monitoring, additional evaluation, sleep study, a drug, a supplement, or a lifestyle recommendation. In some embodiments, the drug is selected from the group consisting of aspirin, progesterone, magnesium sulfate, a cholesterol medication, a heartburn medication, an angiotensin II receptor antagonist, a calcium channel blocker, a diabetes medication, an erectile dysfunction medication, or a small interfering ribonucleic acid (siRNA) based therapy.

In some embodiments, the molecular subtype of preeclampsia is selected from the group consisting of preterm preeclampsia (e.g., pregnant subjects with preeclampsia who delivered before 37 weeks (<37 weeks)). In some embodiments, the molecular subtype of preeclampsia is pre-term preeclampsia (<37 weeks), and the set of biomarkers comprises a genomic locus associated with pre-term preeclampsia (<37 weeks). In some embodiments, the genomic locus associated with pre-term preeclampsia (<37 weeks) is selected from the group consisting of genes listed in Table 21 and genes listed in Table 24. In some embodiments, the pre-term preeclampsia comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks.

In some embodiments, the method further comprises identifying a clinical intervention for the pregnant subject based at least in part on the presence or elevated risk of the molecular subtype of preeclampsia. In some embodiments, the clinical intervention is selected from a plurality of clinical interventions. In some embodiments, the clinical intervention comprises a drug, a supplement, or a lifestyle recommendation. In some embodiments, the drug is selected from the group consisting of aspirin, progesterone, magnesium sulfate, a cholesterol medication, a heartburn medication, an angiotensin II receptor antagonist, a calcium channel blocker, a diabetes medication, and an erectile dysfunction medication.

In some embodiments, the molecular subtype of preeclampsia is selected from the group consisting of 1) preterm preeclampsia with delivery <37 weeks, and 2) term preeclampsia with severe features delivered at 37 weeks or later (>37 weeks). In some embodiments, the genomic locus associated with the 1) preterm preeclampsia with delivery <37 weeks or the 2) term preeclampsia with severe features delivered >37 weeks is selected from the group consisting of genes listed in Table 22. In some embodiments, the pre-term preeclampsia comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the preeclampsia comprises term preeclampsia with severe features, which may be delivered after 37 weeks, delivery after 38 weeks, delivery after 39 weeks, delivery after 40 weeks, delivery at less than 29 weeks, delivery at less than 30 weeks, delivery at less than 31 weeks, delivery after 41 weeks, delivery after 42 weeks, or delivery after 43 weeks.

In some embodiments, the method further comprises identifying a clinical intervention for the pregnant subject based at least in part on the presence or elevated risk of the molecular subtype of preeclampsia. In some embodiments, the clinical intervention is selected from a plurality of clinical interventions. In some embodiments, the clinical intervention comprises a drug, a supplement, or a lifestyle recommendation. In some embodiments, the drug is selected from the group consisting of aspirin, progesterone, magnesium sulfate, a cholesterol medication, a heartburn medication, an angiotensin II receptor antagonist, a calcium channel blocker, a diabetes medication, and an erectile dysfunction medication.

In some embodiments, the molecular subtype of preeclampsia comprises preterm preeclampsia (<37 weeks) without major risk factors. In some embodiments, the genomic locus associated with preterm preeclampsia (<37 weeks) without major risk factors is selected from the group consisting of genes listed in Table 23.

In some embodiments, the pre-term preeclampsia comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the preeclampsia comprises group of composing term preeclampsia with severe features delivered after 37 weeks. In some embodiments, the method further comprises identifying a clinical intervention for the pregnant subject based at least in part on the presence or elevated risk of the molecular subtype of preeclampsia. In some embodiments, the clinical intervention is selected from a plurality of clinical interventions. In some embodiments, the clinical intervention comprises a drug, a supplement, or a lifestyle recommendation. In some embodiments, the drug is selected from the group consisting of aspirin, progesterone, magnesium sulfate, a cholesterol medication, a heartburn medication, an angiotensin II receptor antagonist, a calcium channel blocker, a diabetes medication, and an erectile dysfunction medication.

In some embodiments, the discovery of a molecular subtype of preeclampsia may be improved by applying corrections such as: 1) correction for technical variation, such as computationally stabilizing assay variation; 2) correction for sample-to-sample variation, such as normalizing or correcting using genes (e.g., identified by negative binomial dispersion residuals); 3) correcting for collinearity effects (e.g., due to correlated and biochemically related genes); 4) fetal fraction corrections based on gene correlations to male fetus signals; or a combination thereof. In some embodiments, the preeclampsia normalization or correction genes are selected from the group of genes consisting of KISS1, XAGE2, PSG2, ADAM12, PSG9, CYP19A1, CSH1, HSD17B1, CSH2. KRT7, KRT8, CGA, SVEP2, and IGFBP5.

In some embodiments, the pregnancy-related state is a subtype of gestational diabetes (GDM), and the at least three distinct pregnancy-related states include at least two distinct subtypes of gestational diabetes, at least three distinct subtypes of gestational diabetes, or at least four distinct subtypes of gestational diabetes. In some embodiments, the subtype of gestational diabetes is a molecular subtype of gestational diabetes, and the at least two distinct subtypes of gestational diabetes include at least two distinct molecular subtypes of gestational diabetes, at least three distinct molecular subtypes of gestational diabetes, or at least two distinct molecular subtypes of gestational diabetes. In some embodiments, the subtypes of gestational diabetes mellitus (GDM) belong to two distinctive classification groups. Cases of type A1 GDM (GDMA1) may be managed with diet and exercise, and cases of A2 type GDM (GDMA2) may require pharmacotherapy to manage hypoglycemia.

In some embodiments, the genomic locus associated with type A1 GDM (GDMA1) or A2 type GDM (GDMA2) is selected from the group consisting of genes listed in Table 16, genes listed in Table 17, and genes listed in Table 18. In some embodiments, the set of biomarkers comprises a genomic locus associated with gestational diabetes. In some embodiments, the genomic locus associated with gestational diabetes is selected from the group consisting of PDK4, CSH1, PLAC4, TBCEL, and FBXO7. In some embodiments, the method further comprises identifying a clinical intervention for the pregnant subject based at least in part on the presence or elevated risk of the molecular subtype of gestational diabetes. In some embodiments, the clinical intervention is selected from a plurality of clinical interventions. In some embodiments, the clinical intervention comprises blood glucose surveillance follow-up by 3 hr GTT test, a drug, a supplement, and or a lifestyle recommendation.

Antenatal screening tools may fail to detect up to 70% of pregnant subjects with a growth restricted fetus, thereby illustrating an unmet need in current standard of antenatal care. Odds of still birth increase significantly as gestational age adjusted birth weight decreases from SGA10 (10th percentile) down to SGA1 (1st percentile). In some embodiments, the pregnancy-related state (e.g., pregnancy complication) is a subtype of intrauterine growth restriction or fetal growth restriction, resulting in a baby that is born small for gestational age (SGA). In some embodiments, SGA corresponds to a birth weight at percentiles less than or equal to 1st, 2nd, 3rd, 4th, 5th, 6th, 7th, 8th, 9th, or 10th percentile (e.g., among a general population of baby birth weights). In some embodiments, the subtype of intrauterine or fetal growth restriction is a molecular subtype of intrauterine or fetal growth restriction, and the distinct subtypes of intrauterine or fetal growth restriction include at least two, at least three, at least four, at least five, at least six, at least seven, at least eight, at least nine, or at least ten distinct molecular subtypes of intrauterine or fetal growth restriction (e.g., corresponding to less than or equal to 1st, 2nd, 3rd, 4th, 5th, 6th, 7th, 8th, 9th, or 10th percent baby weight at birth). In some embodiments, the genomic locus is associated with the 3rd baby weight percentile (SGA3), and may be selected from the group consisting of genes listed in Table 19 and genes listed in Table 20.

In some embodiments, the method further comprises identifying a clinical intervention for the pregnant subject based at least in part on the presence or elevated risk of the molecular subtype of intrauterine or fetal growth restriction. In some embodiments, the clinical intervention is selected from a plurality of clinical interventions. In some embodiments, the clinical intervention comprises a drug, a supplement, a lifestyle recommendation, evaluation of fetal biometry using ultrasound examination and doppler monitoring or biophysical profile, evaluation of fetal distress by fetal heart rate monitoring, and medically-indicated preterm delivery.

In some embodiments, the discovery molecular subtype of a subtype of intrauterine or fetal growth restriction may be improved by applying corrections such as: 1) correction for technical variation, such as computationally stabilizing assay variation; 2) correction for sample-to-sample variation, such as normalizing or correcting using genes (e.g., identified by negative binomial dispersion residuals); 3) correcting for collinearity effects (e.g., due to correlated and biochemically related genes); 4) fetal fraction corrections based on gene correlations to male fetus signals; or a combination thereof. In some embodiments, the subtype intrauterine or fetal growth restriction normalization or correction genes are selected from the group consisting of RAP1B and IRAK3.

In some embodiments, the pregnant subject may be provided a clinical care plan which may be tailored to the molecular subtype of intrauterine or fetal growth restriction or molecular subtype of babies born small for gestational age (SGA). The clinical care plan may include growth ultrasound done at 24-28 weeks, umbilical artery dopplers, fetal monitoring such as non-stress test and biophysical profile, scheduling delivery timing such as scheduled induction or c-section to avert still birth, or a combination thereof.

In some embodiments, the set of biomarkers comprises at least 5 distinct genomic loci, at least 10 distinct genomic loci, at least 15 distinct genomic loci, at least 20 distinct genomic loci, at least 25 distinct genomic loci, at least 30 distinct genomic loci, at least 35 distinct genomic loci, at least 40 distinct genomic loci, at least 45 distinct genomic loci, at least 50 distinct genomic loci, at least 100 distinct genomic loci, at least 150 distinct genomic loci, or at least 200 distinct genomic loci.

In some embodiments, the assaying comprises using cell-free ribonucleic acid (cfRNA) molecules derived from the cell-free biological sample to generate transcriptomic data, using transcription products derived from the cell-free biological sample to generate transcription product data, using cell-free deoxyribonucleic acid (cfDNA) molecules derived from the cell-free biological sample to generate genomic data and/or methylation data, using proteins derived from the first cell-free biological sample to generate proteomic data, or using metabolites derived from the first cell-free biological sample to generate metabolomic data.

In some embodiments, the cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, the cell-free biological sample is obtained or derived from the pregnant subject using an ethylenediaminetetraacetic acid (EDTA) collection tube, a cell-free RNA collection tube, or a cell-free deoxyribonucleic acid (DNA) collection tube. In some embodiments, the method further comprises fractionating a whole blood sample of the pregnant subject to obtain the cell-free biological sample. In some embodiments, the assaying comprises a cell-free ribonucleic acid (cfRNA) assay or a metabolomics assay. In some embodiments, the metabolomics assay comprises targeted mass spectroscopy (MS) or an immune assay. In some embodiments, the cell-free biological sample comprises cell-free ribonucleic acid (cfRNA) or urine. In some embodiments, the assaying comprises quantitative polymerase chain reaction (qPCR). In some embodiments, the assaying comprises a home use test configured to be performed in a home setting.

In some embodiments, the trained algorithm determines the presence or elevated risk of the pregnancy-related state of the pregnant subject at a sensitivity of at least about 60%, at least about 65%, at least about 70%, at least about 75%, at least about 80%, at least about 85%, at least about 90%, or at least about 95%. In some embodiments, trained algorithm determines the presence or elevated risk of the pregnancy-related state of the pregnant subject at a positive predictive value (PPV) of at least about 60%, at least about 65%, at least about 70%, at least about 75%, at least about 80%, at least about 85%, at least about 90%, or at least about 95%. In some embodiments, the trained algorithm determines the presence or elevated risk of the pregnancy-related state of the pregnant subject with an Area Under Curve (AUC) of at least about 0.60, at least about 0.65, at least about 0.70, at least about 0.75, at least about 0.80, at least about 0.85, at least about 0.90, or at least about 0.95.

In some embodiments, the pregnant subject is asymptomatic for the pregnancy-related state.

In some embodiments, the trained algorithm is trained using a first set of independent training samples associated with a presence or elevated risk of the pregnancy-related state and a second set of independent training samples associated with an absence or no elevated risk of the pregnancy-related state.

In some embodiments, the method further comprises using the trained algorithm or another trained algorithm to process a set of clinical health data of the pregnant subject to determine the presence or elevated risk of the pregnancy-related state.

In some embodiments, the method further comprises subjecting the cell-free biological sample to conditions that are sufficient to isolate, enrich, or extract a set of ribonucleic (RNA) molecules, deoxyribonucleic acid (DNA) molecules, proteins, or metabolites; and wherein the assaying comprises analyzing the set of RNA molecules, DNA molecules, proteins, or metabolites. In some embodiments, the method further comprises extracting a set of nucleic acid molecules from the cell-free biological sample, and subjecting the set of nucleic acid molecules to sequencing to generate a set of sequencing reads. In some embodiments, the sequencing comprises massively parallel sequencing. In some embodiments, the sequencing comprises nucleic acid amplification. In some embodiments, the nucleic acid amplification comprises polymerase chain reaction (PCR). In some embodiments, the PCR comprises digital PCR. In some embodiments, the digital PCR comprises digital droplet PCR. In some embodiments, the sequencing comprises RNA sequencing. In some embodiments, the RNA sequencing comprises single-molecule RNA sequencing. In some embodiments, the sequencing comprises use of reverse transcription (RT) and polymerase chain reaction (PCR). In some embodiments, the method further comprises using probes configured to selectively enrich the set of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises a genomic locus associated with spontaneous pre-term birth, wherein the genomic locus is selected from the group consisting of genes listed in Table 1, genes listed in Table 2, genes listed in Table 3, genes corresponding to a pathway listed in Table 4, genes listed in Table 9, genes listed in Table 10, and genes listed in Table 11. In some embodiments, the panel of the one or more genomic loci comprises a genomic locus associated with pre-term preeclampsia, wherein the genomic locus is selected from the group consisting of genes listed in Table 5, genes listed in Table 6, genes listed in Table 7, genes listed in Table 8. In some embodiments, the panel of the one or more genomic loci comprises a genomic locus associated with pre-term preeclampsia with delivery less than 37 weeks, or term preeclampsia with severe features, or preterm preeclampsia without severe features, wherein the genomic locus is selected from the group consisting of genes listed in Table 21, genes listed in Table 22, genes listed in Table 23, and genes listed in Table 24. In some embodiments, the panel of the one or more genomic loci comprises a genomic locus associated with the gestational diabetes mellitus type A1 (GDMA1) and type A2 (GDMA2), wherein the genomic locus is selected from the group consisting of genes listed in Table 16, genes listed in Table 17, and genes listed in Table 18.

In some embodiments, the panel of the one or more genomic loci comprises a genomic locus associated with subtype of intrauterine growth restriction or fetal growth restriction or small for gestational age (SGA), wherein the genomic locus is selected from the group consisting of genes listed in Table 19, and genes listed in Table 20. In some embodiments, the panel of the one or more genomic loci comprises at least 5 distinct genomic loci, at least 10 distinct genomic loci, at least 15 distinct genomic loci, at least 20 distinct genomic loci, at least 25 distinct genomic loci, at least 30 distinct genomic loci, at least 35 distinct genomic loci, at least 40 distinct genomic loci, at least 45 distinct genomic loci, at least 50 distinct genomic loci, at least 100 distinct genomic loci, at least 150 distinct genomic loci, or at least 200 distinct genomic loci.

In some embodiments, the cell-free biological sample is processed without nucleic acid isolation, enrichment, or extraction.

In some embodiments, the method further comprises generating an electronic report comprising an indication of the determined presence or elevated risk of the pregnancy-related state.

In some embodiments, the method further comprises determining a likelihood of the determination of the presence or elevated risk of the pregnancy-related state of the pregnant subject.

In some embodiments, the trained algorithm comprises a trained machine learning algorithm. In some embodiments, the trained machine learning algorithm comprises a deep learning algorithm, a support vector machine (SVM), a neural network, a Random Forest, a linear regression model, a logistic regression model, or an ANOVA model.

In some embodiments, the method further comprises processing the set of biomarkers to reduce systematic variations. In some embodiments, reducing the systematic variations comprises using residuals from multivariate linear regression to correct data residuals, performing a ComBat method based on an empirical Bayes approach, and performing a surrogate variables analysis (SVA) correction. In some embodiments, the systematic variations comprise a depth of sequencing per sample, batch effects for individual process operations, use of various raw materials, local outside temperature of sample collection, BMI of the subject, fetal fraction, fetal gestational age at sample collection, or a combination thereof.

In some embodiments, the method further comprises monitoring the presence or elevated risk of the pregnancy-related state, wherein the monitoring comprises assessing the presence or elevated risk of the pregnancy-related state of the pregnant subject at a plurality of time points, wherein the assessing is based at least on the presence or elevated risk of the pregnancy-related state determined at each of the plurality of time points. In some embodiments, a difference in the assessment of the presence or elevated risk of the pregnancy-related state of the pregnant subject among the plurality of time points is indicative of one or more clinical indications selected from the group consisting of (i) a diagnosis of the presence or elevated risk of the pregnancy-related state of the pregnant subject, (ii) a prognosis of the presence or elevated risk of the pregnancy-related state of the pregnant subject, and (iii) an efficacy or non-efficacy of a course of treatment for treating the presence or elevated risk of the pregnancy-related state of the pregnant subject.

In some embodiments, the pregnant subject is in a first trimester of pregnancy, a second trimester of pregnancy, or a third trimester of pregnancy.

In some embodiments, the reference value is determined from pregnant subjects and/or non-pregnant subjects. In some embodiments, processing the set of biomarkers against the reference value comprises determining a difference between the set of biomarkers and the reference value.

In another aspect, the present disclosure provides a method for identifying a presence or susceptibility of a pregnancy-related state of a subject, comprising assaying transcripts and/or metabolites in a cell-free biological sample derived from the subject to detect a set of biomarkers, and analyzing the set of biomarkers with a trained algorithm to determine the presence or susceptibility of the pregnancy-related state. In some embodiments, the method comprises assaying the transcripts in the cell-free biological sample derived from the subject to detect the set of biomarkers. In some embodiments, the transcripts are assayed with nucleic acid sequencing. In some embodiments, the method comprises assaying the metabolites in the cell-free biological sample derived from the subject to detect the set of biomarkers. In some embodiments, the metabolites are assayed with a metabolomics assay.

In another aspect, the present disclosure provides a method for identifying a presence or susceptibility of a pregnancy-related state of a subject, comprising assaying a cell-free biological sample derived from the subject to detect a set of biomarkers, and analyzing the set of biomarkers with a trained algorithm to determine the presence or susceptibility of the pregnancy-related state among a set of at least three distinct pregnancy-related states (e.g., at an accuracy of at least about 80%).

In some embodiments, the pregnancy-related state is selected from the group consisting of pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the pregnancy-related state is a subtype of pre-term birth, and the at least three distinct pregnancy-related states include at least two distinct subtypes of pre-term birth. In some embodiments, the subtype of pre-term birth is a molecular subtype of pre-term birth, and the at least two distinct subtypes of pre-term birth include at least two distinct molecular subtypes of pre-term birth. In some embodiments, the distinct molecular subtypes of pre-term birth comprise a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the pregnancy-related state is a subtype of preeclampsia, and the at least three distinct pregnancy-related states include at least two distinct subtypes of preeclampsia. In some embodiments, the distinct molecular subtypes of preeclampsia comprise a molecular subtype of preeclampsia selected from the group consisting of presence or history of chronic or pre-existing hypertension, presence or history of gestational hypertension, presence or history of mild preeclampsia (e.g., with delivery at greater than 34 weeks gestational age), presence or history of severe preeclampsia (e.g., with delivery at less than 34 weeks gestational age), presence or history of eclampsia, and presence or history of HELLP syndrome.

In some embodiments, the method further comprises identifying a clinical intervention for the subject based at least in part on the presence or susceptibility of the pregnancy-related state. In some embodiments, the clinical intervention is selected from a plurality of clinical interventions. In some embodiments, the method further comprises determining a likelihood of the determination of the susceptibility of the pregnancy-related state of the subject, after which subject can be provided with the clinical intervention. In some embodiments, the clinical intervention comprises a pharmacological, surgical, or procedural treatment to reduce severity, delay, or eliminate the future susceptibility pregnancy-related state of the subject (e.g., aspirin for preeclampsia and steroids for pre-term birth).

In another aspect, the present disclosure provides a method comprising assaying a cell-free biological sample derived from a subject; identifying the subject as having or at risk of having preeclampsia; and upon identifying the subject as having or at risk of having preeclampsia, administering an anti-hypertensive drug to the subject.

In some embodiments, the cell-free biological sample is collected from the subject within a given gestational age interval for detection of a pregnancy-related state. In some embodiments, the given gestational age interval is within about 1 day, about 2 days, about 3 days, about 4 days, about 5 days, about 6 days about 7 days, about 8 days, about 9 days, about 10 days, about 11 days, about 12 days, about 13 days, about 14 days, about 3 weeks, or about 4 weeks from a given gestational age. In some embodiments, the given gestational age is about 0 weeks, about 1 week, about 2 weeks, about 3 weeks, about 4 weeks, about 5 weeks, about 6 weeks, about 7 weeks, about 8 weeks, about 9 weeks, about 10 weeks, about 11 week, about 12 weeks, about 13 weeks, about 14 weeks, about 15 weeks, about 16 weeks, about 17 weeks, about 18 weeks, about 19 weeks, about 20 weeks, about 21 week, about 22 weeks, about 23 weeks, about 24 weeks, about 25 weeks, about 26 weeks, about 27 weeks, about 28 weeks, about 29 weeks, about 30 weeks, about 31 week, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, or about 45 weeks. In some embodiments, the pregnancy-related state comprises one or more of pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, (a) comprises (i) subjecting the cell-free biological sample to conditions that are sufficient to isolate, enrich, or extract a set of ribonucleic (RNA) molecules, deoxyribonucleic acid (DNA) molecules, transcription products (e.g., messenger RNA, transfer RNA, or ribosomal RNA), proteins (e.g., pregnancy-associated proteins corresponding to pregnancy-associated genomic loci or genes), or metabolites, and (ii) analyzing the set of RNA molecules, DNA molecules, proteins, or metabolites using the first assay to generate the first dataset. In some embodiments, the method further comprises extracting a set of nucleic acid molecules from the cell-free biological sample, and subjecting the set of nucleic acid molecules to sequencing to generate a set of sequencing reads, wherein the first dataset comprises the set of sequencing reads. In some embodiments, (b) comprises (i) subjecting the vaginal or cervical biological sample to conditions that are sufficient to isolate, enrich, or extract a population of microbes, and (ii) analyzing the population of microbes using the second assay to generate the second dataset.

In some embodiments, the sequencing is massively parallel sequencing. In some embodiments, the sequencing comprises nucleic acid amplification. In some embodiments, the nucleic acid amplification comprises polymerase chain reaction (PCR). In some embodiments, the PCR comprises digital PCR. In some embodiments, the digital PCR comprises digital droplet PCR. In some embodiments, the sequencing comprises RNA sequencing. In some embodiments, the RNA sequencing comprises single-molecule RNA sequencing. In some embodiments, the sequencing comprises use of simultaneous reverse transcription (RT) and polymerase chain reaction (PCR). In some embodiments, the method further comprises using probes configured to selectively enrich the set of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci.

In some embodiments, the panel of the one or more genomic loci comprises a genomic locus associated with due date. In some embodiments, the panel of the one or more genomic loci comprises a genomic locus associated with gestational age. In some embodiments, the panel of the one or more genomic loci comprises a genomic locus associated with pre-term birth. In some embodiments, the panel of the one or more genomic loci comprises a genomic locus associated with preeclampsia. In some embodiments, the panel of the one or more genomic loci comprises a genomic locus associated with fetal organ development. In some embodiments, the set of biomarkers comprises a genomic locus associated with gestational diabetes mellitus.

In some embodiments, the cell-free biological sample is processed without nucleic acid isolation, enrichment, or extraction.

In some embodiments, the report is presented on a graphical user interface of an electronic device of a user. In some embodiments, the user is the subject.

In some embodiments, the method further comprises determining a likelihood of the determination of the presence or susceptibility of the pregnancy-related state of the subject.

In some embodiments, the trained algorithm comprises a supervised machine learning algorithm. In some embodiments, the supervised machine learning algorithm comprises a deep learning algorithm, a support vector machine (SVM), a neural network, or a Random Forest. In some embodiments, the trained algorithm comprises a differential expression algorithm. In some embodiments, the differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof.

In some embodiments, the method further comprises providing the subject with a therapeutic intervention for the presence or susceptibility of the pregnancy-related state. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points.

In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular subtype of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using the trained algorithm to determine a molecular subtype of the preeclampsia from among a plurality of distinct molecular subtypes of preeclampsia comprising a molecular subtype of preeclampsia selected from the group consisting of history of chronic/pre-existing hypertension, gestational hypertension, mild preeclampsia (e.g., with delivery at >34 weeks), severe preeclampsia (e.g., with delivery at <34 weeks), eclampsia, and HELLP syndrome.

In some embodiments, the method further comprises providing the subject with a therapeutic intervention based at least in part on the risk score indicative of the risk of pre-term birth. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist, COX inhibitors, calcium channel blockers (i.e. nifedipine), magnesium sulfate, betamethasone, NSAIDs for tocolysis e.g. indomethacin.

In some embodiments, the method further comprises providing the subject with a therapeutic intervention based at least in part on the risk score indicative of the risk of preeclampsia. In some embodiments, the therapeutic intervention comprises antihypertensive drug therapy (such as but not limited to hydralazine, labetalol, nifedipine, and sodium nitroprusside), management or prevention of seizures (such as but not limited to magnesium sulfate, phenytoin, and diazepam), or prevention by low-dose aspirin therapy (e.g., 160 mg per day or less) to reduce the incidence of preeclampsia.

In some embodiments, the method further comprises monitoring the risk of pre-term birth, wherein the monitoring comprises assessing the risk of pre-term birth of the subject at a plurality of time points, wherein the assessing is based at least on the risk score indicative of the risk of pre-term birth determined in (b) at each of the plurality of time points.

In some embodiments, the method further comprises monitoring the risk of preeclampsia, wherein the monitoring comprises assessing the risk of preeclampsia of the subject at a plurality of time points, wherein the assessing is based at least on the risk score indicative of the risk of preeclampsia determined in (b) at each of the plurality of time points.

In some embodiments, the method further comprises refining the risk score indicative of the risk of preeclampsia of the subject by performing one or more subsequent clinical tests for the subject, and processing results from the one or more subsequent clinical tests using a trained algorithm to determine an updated risk score indicative of the risk of preeclampsia of the subject. In some embodiments, the one or more subsequent clinical tests comprise an ultrasound imaging or a blood test. In some embodiments, the risk score comprises a likelihood of the subject having preeclampsia within a pre-determined duration of time.

In some embodiments, the method further comprises refining the risk score indicative of the risk of preeclampsia of the subject by performing one or more subsequent clinical tests for the subject, and processing results from the one or more subsequent clinical tests using a trained algorithm to determine an updated risk score indicative of the risk of preeclampsia of the subject. In some embodiments, the one or more subsequent clinical tests comprise an ultrasound imaging or a blood test. In some embodiments, the risk score comprises a likelihood of the subject having a preeclampsia within a pre-determined duration of time.

In some embodiments, the pre-determined duration of time is about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

In some embodiments, the method further comprises providing the subject with a therapeutic intervention for the presence or susceptibility of the pregnancy-related state. In some embodiments, therapeutic intervention comprises a progesterone treatment such as hydroxyprogesterone caproate (e.g., 17-alpha hydroxyprogesterone caproate (17-P), LPCN 1107 from Lipocine, Makena from AMAG Pharma), a vaginal progesterone, or a natural progesterone IVR product (e.g., DARE-FRT1 (JNP-0301) from Juniper Pharma); a prostaglandin F2 alpha receptor antagonist (e.g., OBE022 from ObsEva); or a beta2-adrenergic receptor agonist (e.g., bedoradrine sulfate (MN-221) from MediciNova). Therapeutic interventions may be described by, for example, “WHO Recommendations on Interventions to Improve Preterm Birth Outcomes,” ISBN 9789241508988, World Health Organization, 2015, which is hereby incorporated by reference in its entirety. In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points. In some embodiments, a difference in the assessment of the presence or susceptibility of the pregnancy-related state of the subject among the plurality of time points is indicative of one or more clinical indications selected from the group consisting of (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular subtype of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), history of cervical insufficiency, and presence or history of preterm prelabor rupture of membrane (PPROM).

In some embodiments, the method further comprises stratifying the preeclampsia by using the trained algorithm to determine a molecular subtype of the preeclampsia from among a plurality of distinct molecular subtypes of preeclampsia. In some embodiments, the plurality of distinct molecular subtypes of preeclampsia comprises a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In some embodiments, the method further comprises analyzing the set of biomarkers with a trained algorithm. In some embodiments, the health or physiological condition is selected from the group consisting of pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). In some embodiments, the set of biomarkers comprises a genomic locus associated with due date, gestational age, pre-term birth, preeclampsia, fetal organ development, or gestational diabetes mellitus. In some embodiments, the method further comprises selecting a therapeutic intervention for the health or physiological condition of the fetus of the pregnant subject or of the pregnant subject, based at least in part on the set of biomarkers. In some embodiments, the therapeutic intervention is selected from among a plurality of therapeutic interventions. In some embodiments, the therapeutic intervention is selected based at least in part on a molecular subtype of the health or physiological condition determined based at least in part on the set of biomarkers.

In some embodiments, the therapeutic intervention is selected based on molecular subtype of preterm birth related to collagen-containing extracellular matrix pathway and comprising drug selected from group of collagen modulating therapeutics like thrombin, Y-27632, TNF alpha and indomethacin.

In some embodiments, the therapeutic intervention is selected based on molecular subtype of preterm birth related to extracellular matrix (ECM) pathway and comprising a therapeutic agent, wherein the therapeutic agent is an oncofetal fibronectin modulating agent. In some embodiments, the therapeutic agent is a glucocorticoid inhibitor. In some embodiments, the therapeutic agent is dexamethasone or betamethasone. In some embodiments, the therapeutic agent is cycloheximide.

In some embodiments, the therapeutic intervention is selected based on molecular subtype of preterm birth related to endoplasmic reticulum (ER) lumen pathway associated with endoplasmic reticulum stress induced by oxidative stress in decidual cells and comprising drug selected from group of ER stress inhibitors 4-phenylbutyric acid (4-PBA) and tauroursodeoxycholic acid (TUDCA).

In some embodiments, the therapeutic intervention is selected based on molecular subtype of preterm birth related inflammation pathways and comprising drug selected from group of non-specific NF-κB inhibitors, TLR4 antagonists, TNF-α biologics, CTHE (novel class of anti-inflammatory drugs): p38 MAPK inhibitors (SKF-86002, SB202190 and SB239063), IKK complex inhibitors (NBNI, parthenolide, and, TPCA-1), or TAK1 inhibitors (5z-7-oxozeaenol (OxZnl)).

In some embodiments, the therapeutic intervention is selected based on molecular subtype of preterm birth related insulin growth-factor transport pathway and comprising a therapeutic agent selected from a group of Metformin, Insulin-like growth factor 1 (IGF-1), Insulin-Like Growth Factor Binding Protein-3 (IGFBP-3), and modulators of glucose transporters (GLUT3, GLUT8 and GLUT9). In some embodiments, the therapeutic agent is metformin. In some embodiments, the therapeutic agent is IGF-1. In some embodiments, the therapeutic agent is IGFBP-3. In some embodiments, the therapeutic agent is a modulator of GLUT3. In some embodiments, the therapeutic agent is a modulator of GLUT8. In some embodiments, the therapeutic agent is a modulator of GLUT9.

In some embodiments, the therapeutic intervention is selected based on molecular subtype of preterm birth related metabolism of amino acids and derivatives and comprising therapeutic agent, wherein the therapeutic agent is an endogenous metabolic modulator (EMM).

In some embodiments, the health or physiological condition comprises preeclampsia. In some embodiments, the therapeutic intervention for the preeclampsia comprises a drug, a supplement, remote patient monitoring or a lifestyle recommendation. In some embodiments, the drug is selected from the group consisting of aspirin, progesterone, magnesium sulfate, a cholesterol medication (such as pravastatin), a heartburn medication (such as esomeprazole), an angiotensin II receptor antagonist (such as losartan), a calcium channel blocker (such as nifedipine), a diabetes medication (such as myo-inositol, metformin, glyburide, and liraglutide), and an erectile dysfunction medication (such as sildenafil citrate). In some embodiments, the supplement is selected from the group consisting of calcium, vitamin D, vitamin B3, and DHA. In some embodiments, the lifestyle recommendation is selected from the group consisting of exercise, nutrition counseling, meditation, stress relief, weight loss or maintenance, improving sleep quality with sleep study, and prescription of continuous positive airway pressure for treatment of obstructive sleep apnea. In some embodiments, the therapeutic intervention for the preeclampsia is selected from a therapeutic intervention (e.g., treatment or prophylaxis) as disclosed in “WHO recommendations: Prevention and treatment of pre-eclampsia and eclampsia,” World Health Organization, ISBN 9789241548335, World Health Organization, 2011, which is incorporated by reference herein in its entirety. In some embodiments, the therapeutic intervention for the preeclampsia is selected from a therapeutic intervention (e.g., treatment or prophylaxis) as disclosed in “Summary of recommendations: Prevention and treatment of pre-eclampsia and eclampsia,” World Health Organization, WHO reference number WHO/RHR/11.30, World Health Organization, 2011, which is incorporated by reference herein in its entirety. In some embodiments, the therapeutic intervention for the preeclampsia is selected from a therapeutic intervention (e.g., treatment or prophylaxis) as disclosed in “WHO recommendations: Drug treatment for severe hypertension in pregnancy,” World Health Organization, ISBN 9789241550437, World Health Organization, 2018, which is incorporated by reference herein in its entirety.

In some embodiments, the therapeutic intervention is selected based on molecular subtype of preeclampsia related to early placentation steps responsible for modulating the vasodilatory mediators and inhibiting vascular remodeling, platelet aggregation, and platelet adhesion and comprising drug selected from group of direct-acting vasodilators (hydralazine, minoxidil, nitrates, nitroprusside); calcium channel blockers (verapamil, diltiazem, nifedipine, amlodipine); an antagonist of the renin-angiotensin-aldosterone system (angiotensin receptor blockers, angiotensin-converting-enzyme inhibitors); Beta-2 receptor agonist (salbutamol, terbutaline); ostsynaptic alpha-1 receptor antagonist (prazosin, phenoxybenzamine, phentolamine); centrally acting alpha-2 receptor agonist (clonidine, α-methyldopa); centrally acting alpha-2 receptor agonist (clonidine, α-methyldopa); Centrally acting alpha-2 receptor agonist (clonidine, α-methyldopa).

In some embodiments, the therapeutic intervention is selected based on molecular subtype of preeclampsia related to the molecular subtype of PE associated with keratinocyte endothelium pathway and comprising drug selected from group of proton pump inhibitors (PPI): omeprazole, esomeprazole, pantoprazole, rabeprazole, or lansoprazole.

In some embodiments, the health or physiological condition comprises pre-term birth. In some embodiments, the therapeutic intervention for the pre-term birth comprises a drug, a supplement, a lifestyle recommendation, a cervical cerclage, a cervical pessary, or electrical contraction inhibition. In some embodiments, the drug is selected from the group consisting of progesterone, erythromycin, a tocolytic medication (such as indomethacin), a corticosteroid, a vaginal flora (such as clindamycin and metronidazole), and an antioxidant (such as N-acetylcysteine). In some embodiments, the supplement is selected from the group consisting of calcium, vitamin D, and a probiotic (such as lactobacillus). In some embodiments, the lifestyle recommendation is selected from the group consisting of exercise, nutrition counseling, meditation, stress relief, weight loss or maintenance, and improving sleep quality. In some embodiments, the therapeutic intervention for the pre-term birth is selected from a therapeutic intervention (e.g., treatment or prophylaxis) as disclosed “WHO Recommendations on Interventions to Improve Preterm Birth Outcomes,” ISBN 9789241508988, World Health Organization, 2015, which is incorporated by reference herein in its entirety.

In some embodiments, the health or physiological condition comprises gestational diabetes mellitus (GDM). In some embodiments, the therapeutic intervention for the GDM comprises a drug, a supplement, or a lifestyle recommendation. In some embodiments, the drug is selected from the group consisting of insulin and a diabetes medication (such as myo-inositol, metformin, glyburide, and liraglutide). In some embodiments, the supplement is selected from the group consisting of vitamin D, choline, probiotics, and DHA. In some embodiments, the lifestyle recommendation is selected from the group consisting of exercise, nutrition counseling, meditation, stress relief, weight loss or maintenance, and improving sleep quality. In some embodiments, the therapeutic intervention for the gestational diabetes mellitus (GDM) is selected from a therapeutic intervention (e.g., treatment or prophylaxis) as disclosed “Diagnostic criteria and classification of hyperglycaemia first detected in pregnancy,” WHO reference number WHO/NMH/MND/13.2, World Health Organization, 2013, which is incorporated by reference herein in its entirety.

In some embodiments, the therapeutic intervention is selected based on molecular subtype of gestational diabetes mellitus (GDM) related to the molecular subtype of GDM associated placenta deterioration, placenta insufficiency, placenta failure, placenta dysfunction, premature ageing, calcification and comprising drug selected from group of sildenafil citrate, tempol ((superoxide dismutase dismutase), resveratrol, melatonin, sofalcone, statins, metformin, or [Leu27] insulin-like growth factor-II (IGF-II).

In some embodiments, the subtypes of gestational diabetes mellitus GDM may fall into two distinctive classification groups. Cases of type A1 GDM (GDMA1) may be managed with diet and exercise, and cases of A2 type GDM (GDMA2) may require pharmacotherapy to manage hypoglycemia.

In some embodiments, the therapeutic intervention is selected based on molecular subtype of gestational diabetes mellitus (GDM) related to the molecular subtype of GDM associated with mediated hyperglycemic memory and comprising drug selected from group pravastatin, aminoguanidine, rosiglitazone, grape seed proanthocyanidins extracts (GSPE), hesperidin, epalrestat, pyridoxamine, telmisartan, metformin, and pioglitazone.

In some embodiments, the therapeutic intervention is selected based on molecular subtype of gestational diabetes mellitus (GDM) related to the molecular subtype of GDM associated with adaptive immune system and antigen-specific pathways and comprising drug selected from group azathioprine, mycophenolate mofeti, otelixizumab, teplizumab, GAD65, DiaPep277, anti-CD20 mAb, Rapamycin/IL-2, sulfonylureas, metformin, TZDs, dipeptidyl peptidase-4 Inhibitors, sodium-glucose cotransporter 2 inhibitors, diacerein, salsalate, or GLP-1 RAs.

In some embodiments, the therapeutic or clinical intervention is selected based at least in part on a molecular subtype of intrauterine growth restriction or fetal growth restriction associated with placenta deterioration, placenta insufficiency, placenta failure, placenta dysfunction, or a combination thereof. In some embodiments, the clinical intervention comprises a drug, a supplement, a lifestyle recommendation, evaluation of fetal biometry (e.g., using ultrasound examination and/or Doppler monitoring), evaluation of fetal distress (e.g., by antepartum testing such as fetal heart rate monitoring), a medically-indicated preterm delivery (e.g., by labor induction or Caesarian section), or a combination thereof.

In some embodiments, the panel of the one or more genomic loci comprises a genomic locus associated with subtype of PAPPA2+ preeclampsia. In some embodiments, the genomic locus is selected from the group consisting of genes listed in Table 25. In some embodiments, the panel of the one or more genomic loci comprises at least 5 distinct genomic loci, at least 10 distinct genomic loci, at least 15 distinct genomic loci, at least 20 distinct genomic loci.

In some embodiments, the panel of the one or more genomic loci comprises a genomic locus associated with preeclampsia in samples in the lower three quartiles of PAPPA2 expression. In some embodiments, the genomic locus is selected from the group consisting of genes listed in Table 26. In some embodiments, the panel of the one or more genomic loci comprises at least 5 distinct genomic loci, at least 10 distinct genomic loci, at least 15 distinct genomic loci, at least 20 distinct genomic loci.

In some embodiments, the panel of the one or more genomic loci comprises a genomic locus associated with hypertensive disorders of pregnancy in samples in the lower three quartiles of PAPPA2 expression. In some embodiments, the genomic locus is selected from the group consisting of genes listed in Table 27. In some embodiments, the panel of the one or more genomic loci comprises at least 5 distinct genomic loci, at least 10 distinct genomic loci, at least 15 distinct genomic loci, at least 20 distinct genomic loci.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a computer system that is programmed or otherwise configured to implement methods provided herein.

FIGS. 2A-2C show an example of receiver-operator characteristic (ROC) curves for a predictive model using differentially expressed genes identified by gene discovery (0A), wherein the model was validated by leave-one-out cross-validation (LOOCV), including cross-validating in site 1 with a mean area-under-the-curve (AUC) for the ROC curve of 0.77 (FIG. 2B) and testing in sites 2, 3, and 4 with a mean AUC of 0.79 (FIG. 2C).

FIG. 2D shows an example of identifying pathways in top pre-term birth marker genes via gene set enrichment analysis for gene ontology and pathways for collagen-containing extracellular matrix in samples from mothers that deliver preterm.

FIG. 3 shows an example of identifying pathways in late miscarriage or early pre-term birth via gene set enrichment analysis for gene ontology and pathways for genes set related to basement membrane and endoplasmic reticulum lumen in samples from mothers that deliver before gestational age of 25 weeks.

FIG. 4 shows a distribution of 242 collected samples at gestational age of sample collection, and gestational age at delivery.

FIG. 5A shows a receiver-operator characteristic (ROC) curve for sPTB (delivery <35 weeks of GA).

FIG. 5B shows the probability assigned by the sPTB model (delivery <35 weeks of GA) to each sample. A value of 0 corresponds to a 0% probability of a sample being classified as a sPTB case, and a value of 1 corresponds to a 100% probability of a sample being classified as a sPTB case. Samples are separated by each true label (cases in orange, controls in blue).

FIG. 5C shows a receiver-operator characteristic (ROC) curve for sPTB (delivery <25 weeks of GA).

FIG. 5D shows the probability assigned by the sPTB model (delivery <25 weeks of GA) to each sample. A value of 0 corresponds to a 0% probability of a sample being classified as a sPTB case, and a value of 1 corresponds to a 100% probability of a sample being classified as a sPTB case. Samples are separated by each true label (cases in orange, controls in blue).

FIG. 6 shows the demographic and clinical data metrics for a preeclampsia observational cohort, including 2,701 healthy participants and 335 participants diagnosed with preeclampsia.

FIG. 7 shows the demographic and clinical data for a cohort with a cutoff for preeclampsia-diagnosed subjects with delivery before 38 weeks, including 2,690 healthy participants and 199 participants who were diagnosed with preeclampsia and delivered before 38 weeks of gestational age.

FIG. 8 shows the demographic and clinical data for a cohort with a cutoff for preeclampsia-diagnosed subjects with delivery before 37 weeks, including 2,780 healthy participants and 109 participants who were diagnosed with preeclampsia and delivered before 37 weeks of gestational age.

FIG. 9A shows an example of systemic variance in cell-free RNA (cfRNA) next-generation sequencing (NGS) data associated with seasonal changes for gestational- and placenta-associated genes moving in the opposite direction as the immune cellular genes.

FIG. 9B shows an example of a high correlation between prediction of outside local temperature by gene modeling and actual temperatures recorded by weather station close to the blood collection site.

FIG. 10A shows an example of systemic variance in cfRNA NGS data associated with the time of blood draw/collection.

FIG. 10B shows a quantile-quantile (QQ) plot for differential expression between early morning and afternoon groups associated with a circadian clock.

FIG. 11A shows the discovery rate for genes by Sure Independence Screening in a repeated cross validation in log 2 counts per million (CPM) reads space.

FIG. 11B shows the discovery rate for genes by Sure Independence Screening in a repeated cross validation in log 2 raw count reads space.

FIG. 12A shows features discovered by correction in multiple spaces and thresholding by Akaike information criterion (AIC) for preterm preeclampsia cases with delivery at less than 38 weeks from down sampled counts with clinical factors.

FIG. 12B shows examples of features discovered by correction in multiple spaces and thresholding by AIC for preterm preeclampsia cases with delivery at less than 37 weeks with body mass index (BMI) and blood pressure (BP) as the only clinical factors.

FIG. 13 shows an example of an area-under-the-curve (AUC) for the ROC curve values with mean at 0.83 for a model predicting preterm preeclampsia cases with deliveries at less than 37 weeks of gestation age.

FIG. 14 shows a quantile-quantile (QQ) plot for Spearman and DESeq2 differential gene expression analyses for differentially expressed genes in spontaneous preterm cases delivered before 35 weeks of gestational age. QQ plot for Spearman ranked differential gene expression is on the left, and DESeq2 differential gene expression is on the right.

FIG. 15 shows a quantile-quantile (QQ) plot for DESeq2 differential gene expression analyses for differentially expressed genes in spontaneous preterm cases delivered before 37 weeks of gestational age.

FIG. 16 shows a quantile-quantile (QQ) plot for DESeq2 differential gene expression analyses for differentially expressed genes in gestational diabetes (GDM) cases.

FIG. 17 shows a quantile-quantile (QQ) plot for DESeq2 differential gene expression analyses for differentially expressed genes in gestational diabetes type A1 (GDMA1) cases.

FIG. 18 shows a quantile-quantile (QQ) plot for DESeq2 differential gene expression analyses for differentially expressed genes in gestational diabetes type A2 (GDMA2) cases.

FIG. 19 shows the effect size of newborn baby weight at birth on predictive modeling at different percentile cut off values. The effect size (Cohen's d) peaks at a baby weight percentile cut off of 3% for SGA (vertical line).

FIG. 20 shows the distribution of the most frequent predictive gene features identified with 60 fold cross-validated feature discovery for the newborn babies with fetal growth restriction falling within the 3rd baby weight percentile, indicating small for gestational age (SGA3). Only genes with discovery rates >15% are shown.

FIGS. 21A-21B show the correlation structure of gene features and correction features before (FIG. 21A) and after (FIG. 21B) gene corrections for genes predictive of SGA3.

FIG. 22 shows comparison of PAPPA2 expression differences across 8 different clinical groups. Each cell is colored by PAPPA2 effect size and an indication of statistical significance by multiple testing corrected Mann Whitney U. n.s., not significant, * p<0.05, ** p<0.01, *** p<0.001. ptPE, preeclampsia with delivery <37 weeks; termPE, preeclampsia with delivery >=37 weeks; severe, with severe features; not_severe, without severe features; diag_lt37, diagnosed before 37 weeks; diag_gte37, diagnosed at or after 37 weeks; exclpp/unk, excludes postpartum or unknown timing.

FIGS. 23A-23C show quantile-quantile plots for differential gene expression measured by Mann-Whitney U ranked statistics. FIG. 23A shows differential gene expression for PAPPA2+PE samples compared to all other cohort training samples. FIG. 23B shows PAPPA2+ preeclampsia compared to PAPPA2− preeclampsia. FIG. 23C shows a Q-Q plot of observed p-value and expected p-value from a differential gene expression analyses in samples with no major risk factors.

FIG. 24 shows a comparison of PAPPA2 expression differences split by presence (MRF) or absence (NMRF) of major risk factors, in the PAPPA2+(ptPE) and PAPPA2− (PE_not_ptPE) preeclampsia and in gestational hypertension (GHTN) and controls without any hypertensive disorders of pregnancy (controls). Each cell is colored by PAPPA2 effect size and an indication of statistical significance by multiple testing corrected Mann Whitney U. n.s., not significant, * p<0.05, ** p<0.01, *** p<0.001.

FIGS. 25A-25B show normalized PAPPA2 expression as a function of gestational age at delivery (Delivery GA). FIG. 25A includes all training samples, PAPPA2+ are shown in orange with plus signs indicating individuals with major risk factors and dots indicating individuals without major risk factors, blue color contour is all PAPPA2− samples. Linear fit for each group is overlaid, significance of slope is shown in legend. FIG. 25B shows all training samples without major risk factors, PAPPA2+ are shown in orange and PAPPA2− in blue. Linear fit for each group is overlaid, and significance of slope is shown in legend.

FIGS. 26A-26B show quantile-quantile plots for differential gene expression after removing the upper quartile of PAPPA2 expression measured by Mann-Whitney U ranked statistics. FIG. 26A shows samples with diagnosis of preeclampsia against all others. FIG. 26B shows samples with gestational hypertension against all others.

FIGS. 27A-27B show Kaplan-Meier curves for different subpopulations in training and validation. 27A shows proportion of samples classified as positive using cross-validation in the training data when split into groups of PAPPA2+ preeclampsia delivered before or at 35 weeks, PAPPA2+ preeclampsia delivered before 37 weeks, PAPPA2− preeclampsia, gestational hypertension and controls without hypertensive disorders of pregnancy. 27B shows proportion of samples classified as positive in the validation data when split into groups of PAPPA2+ preeclampsia delivered before or at 35 weeks, PAPPA2+ preeclampsia delivered before 37 weeks, PAPPA2− preeclampsia, gestational hypertension and controls without hypertensive disorders of pregnancy.

FIGS. 28A-28C show receiver-operator characteristic (ROC) curves for train and validation across different subpopulations. FIG. 28A shows train and test performance for predicting PAPPA2+ preeclampsia in samples without major risk factors. FIG. 28B shows train and test performance for predicting PAPPA2+ preeclampsia in samples without major risk factors and delivering before or at 35 weeks. FIG. 28C shows train and test performance for predicting PAPPA2+ preeclampsia in samples without major risk factors and advanced maternal age (35 years or older).

FIG. 29 shows a CONSORT diagram for studies in which molecular data is analyzed for association with clinical phenotypes, such that pregnancy-related disease states can be defined and assessed at a molecular level.

FIG. 30 shows an overlay of effect size of PAPPA2+ and PAPPA2− cases with clinical phenotypes.

FIGS. 31A-31B show Cohen's d effect size of normalized PAPPA2 for different subpopulations. FIG. 31A shows Cohen's d effect size for PAPPA2+ samples, including MRF-cases and MRF+ cases. FIG. 31B shows Cohen's d effect size for samples split by PAPPA2 status (PAPPA2+, PAPPA2− or Non HDP) for all samples for a single gene (PAPPA2), for no major risk factor samples for a single gene (PAPPA2) and for no major risk factors for the genes-only classifier using 6 predictive genes (PAPPA2, CD163, VSIG4, ADAM12, XAGE2, KISS1).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a pregnant female subject. The subject can be a woman having a fetus (or multiple fetuses) or suspected of having the fetus (or multiple fetuses). The subject can be a person that is pregnant or is suspected of being pregnant. The subject may be displaying a symptom(s) indicative of a health or physiological state or condition of the subject, such as a pregnancy-related health or physiological state or condition of the subject. As an alternative, the subject can be asymptomatic with respect to such health or physiological state or condition.

The term “pregnancy-related state,” as used herein, generally refers to any health, physiological, and/or biochemical state or condition of a subject that is pregnant or is suspected of being pregnant, or of a fetus (or multiple fetuses) of the subject. Examples of pregnancy-related states include, without limitation, pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some situations, the pregnancy-related state is not associated with the health or physiological state or condition of a fetus (or multiple fetuses) of the subject.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a vaginal sample (e.g., a vaginal swab), or a cervical sample (e.g., a cervical swab).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Every year, about 15 million pre-term births are reported globally. Pre-term birth may affect as many as about 10% of pregnancies, of which the majority are spontaneous pre-term births. Currently, there may be no meaningful, clinically actionable diagnostic screenings or tests available for many pregnancy-related complications such as pre-term birth. However, pregnancy-related complications such as pre-term birth are a leading cause of neonatal death and of complications later in life. Further, such pregnancy-related complications can cause negative health effects on maternal health. Thus, to make pregnancy as safe as possible, there exists a need for rapid, accurate methods for identifying and monitoring pregnancy-related states that are non-invasive and cost-effective, toward improving maternal and fetal health.

Current tests for prenatal care may be in inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period can be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate and affordable non-invasive diagnostic methods for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods, systems, and kits for identifying or monitoring pregnancy-related states by processing cell-free biological samples obtained from or derived from subjects (e.g., pregnancy female subjects). Cell-free biological samples (e.g., plasma samples) obtained from subjects may be analyzed to identify the pregnancy-related state (which may include, e.g., measuring a presence, absence, or quantitative assessment (e.g., risk) of the pregnancy-related state). Such subjects may include subjects with one or more pregnancy-related states and subjects without pregnancy-related states. Pregnancy-related states may include, for example, pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). In some embodiments, pregnancy-related states are not associated with the health of a fetus. In some embodiments, pregnancy-related states include neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea) and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

### Assaying Cell-Free Biological Samples

The cell-free biological samples may be obtained or derived from a human subject (e.g., a pregnant female subject). The cell-free biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a pregnancy-related state (e.g., a pregnancy-related complication), from a subject that is suspected of having a pregnancy-related state (e.g., a pregnancy-related complication), or from a subject that does not have or is not suspected of having the pregnancy-related state (e.g., a pregnancy-related complication). The pregnancy-related state may comprise a pregnancy-related complication, such as pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine growth restriction, fetal growth restriction, small for gestational age (SGA), macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). The pregnancy-related state may comprise a pre-term birth (e.g., spontaneous pre-term birth or medically-indicated pre-term birth), full-term birth, normal fetal development stages or states (e.g., normal fetal organ function or development), or absence of a pregnancy-related complication (e.g., pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine growth restriction, fetal growth restriction, small for gestational age (SGA), macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). The pregnancy-related state may comprise a quantitative assessment of pregnancy such as gestational age (e.g., measured in days, weeks or months) or due date (e.g., expressed as a predicted or estimated calendar date or range of calendar dates). The pregnancy-related state may comprise a quantitative assessment of a pregnancy-related complication such as a likelihood, a susceptibility, or a risk (e.g., expressed as a probability, a relative probability, an odds ratio, or a risk score or risk index) of the pregnancy-related complication (e.g., pre-term birth (e.g., spontaneous pre-term birth or medically-indicated pre-term birth), full-term birth, gestational age, due date (e.g., due date for an unborn baby or fetus of a subject), onset of labor, cholestasis, oligohydramnios, polyhydramnios, pregnancy-related hypertensive disorders (e.g., preeclampsia), eclampsia, HELLP syndrome, gestational diabetes, a congenital disorder of a fetus of the subject, ectopic pregnancy, spontaneous abortion, stillbirth, antepartum fetal demise, intrapartum fetal demise, intrauterine fetal demise, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, prelabor preterm rupture of membranes, placenta accreta spectrum disorders and placental abruption, placenta previa (placenta covering the cervix), intrauterine growth restriction, fetal growth restriction, small for gestational age (SGA), macrosomia (large fetus for gestational age), neonatal conditions (e.g., neonatal alloimmune thrombocytopenia, neonatal autoimmune thrombocytopenia, fetal genetic syndromes, inborn errors of metabolism, anemia, apnea, bradycardia and other heart defects, bronchopulmonary dysplasia or chronic lung disease, diabetes, gastroschisis (e.g., abdominal wall defects including omphalocele and others), hydrocephaly, hyperbilirubinemia, hypocalcemia, hypoglycemia, intraventricular hemorrhage, jaundice, necrotizing enterocolitis, patent ductus arteriosus, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development)). For example, the pregnancy-related state may comprise a likelihood or susceptibility of an onset of labor in the future (e.g., within about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

The cell-free biological sample may be taken before and/or after treatment of a subject with the pregnancy-related complication. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a pregnancy-related state (e.g., pregnancy-related complication) for which a definitive positive or negative diagnosis is not available via clinical tests. The sample may be taken from a subject suspected of having a pregnancy-related complication. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a pregnancy-related complication due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, using transcription products (e.g., messenger RNA, transfer RNA, or ribosomal RNA) derived from the cell-free biological sample to generate transcription product data, cell-free deoxyribonucleic acid (cfDNA) molecules suitable for assaying to generate genomic data and/or methylation data, proteins (e.g., pregnancy-associated proteins corresponding to pregnancy-associated genomic loci or genes) suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more cell-free biological samples of a subject for downstream assaying using one or more suitable assays.

After obtaining a cell-free biological sample from the subject, the cell-free biological sample may be processed to generate datasets indicative of a pregnancy-related state of the subject. For example, a presence, absence, or quantitative assessment of nucleic acid molecules of the cell-free biological sample at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins (e.g., corresponding to pregnancy-associated genomic loci or genes), and/or metabolome data comprising quantitative measures of a panel of pregnancy-related state-associated metabolites may be indicative of a pregnancy-related state. Processing the cell-free biological sample obtained from the subject may comprise (i) subjecting the cell-free biological sample to conditions that are sufficient to isolate, enrich, or extract a plurality of nucleic acid molecules, proteins (e.g., pregnancy-associated proteins corresponding to pregnancy-associated genomic loci or genes), and/or metabolites, and (ii) assaying the plurality of nucleic acid molecules, proteins, and/or metabolites to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the pregnancy-related state. For example, the sequence reads may be aligned to one or more reference genomes (e.g., a genome of one or more species such as a human genome). The aligned sequence reads may be quantified at one or more genomic loci to generate the datasets indicative of the pregnancy-related state. For example, quantification of sequences corresponding to a plurality of genomic loci associated with pregnancy-related states may generate the datasets indicative of the pregnancy-related state.

The cell-free biological sample may be processed without any nucleic acid extraction. For example, the pregnancy-related state may be identified or monitored in the subject by using probes configured to selectively enrich nucleic acid (e.g., RNA or DNA) molecules corresponding to the plurality of pregnancy-related state-associated genomic loci. The probes may be nucleic acid primers. The probes may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct pregnancy-related state-associated genomic loci or genomic regions. The plurality of pregnancy-related state-associated genomic loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) selected from the group consisting of ACTB, ADAM12, ALPP, ANXA3, APLF, ARGI, AVPR1A, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-A S1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAP1GAP, RGS18, RPL23AP7, S100A8, S100A9, S100P, SERPINA7, SLC2A2, SLC38A4, SLC4A1, TBC1D15, VCAN, VGLL1, B3GNT2, COL24A1, CXCL8, and PTGS2. The pregnancy-related state-associated genomic loci or genomic regions may be associated with gestational age, pre-term birth, due date, onset of labor, or other pregnancy-related states or complications, such as the genomic loci described by, for example, Ngo et al. (“Noninvasive blood tests for fetal development predict gestational age and preterm delivery,” Science, 360(6393), pp. 1133-1136, 8 Jun. 2018), which is hereby incorporated by reference in its entirety.

The probes may be nucleic acid molecules (e.g., RNA or DNA) having sequence complementarity with nucleic acid sequences (e.g., RNA or DNA) of the one or more genomic loci (e.g., pregnancy-related state-associated genomic loci). These nucleic acid molecules may be primers or enrichment sequences. The assaying of the cell-free biological sample using probes that are selective for the one or more genomic loci (e.g., pregnancy-related state-associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., pregnancy-related state-associated genomic loci) to generate the data indicative of the pregnancy-related state. For example, quantification of array hybridization or polymerase chain reaction (PCR) corresponding to a plurality of genomic loci (e.g., pregnancy-related state-associated genomic loci) may generate data indicative of the pregnancy-related state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process cell-free biological samples of a subject. For example, a first assay may be used to process a first cell-free biological sample obtained or derived from the subject to generate a first dataset; and based at least in part on the first dataset, a second assay different from the first assay may be used to process a second cell-free biological sample obtained or derived from the subject to generate a second dataset indicative of the pregnancy-related state. The first assay may be used to screen or process cell-free biological samples of a set of subjects, while the second or subsequent assays may be used to screen or process cell-free biological samples of a smaller subset of the set of subjects. The first assay may have a low cost and/or a high sensitivity of detecting one or more pregnancy-related states (e.g., pregnancy-related complication), that is amenable to screening or processing cell-free biological samples of a relatively large set of subjects. The second assay may have a higher cost and/or a higher specificity of detecting one or more pregnancy-related states (e.g., pregnancy-related complication), that is amenable to screening or processing cell-free biological samples of a relatively small set of subjects (e.g., a subset of the subjects screened using the first assay). The second assay may generate a second dataset having a specificity (e.g., for one or more pregnancy-related states such as pregnancy-related complications) greater than the first dataset generated using the first assay. As an example, one or more cell-free biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process cell-free biological samples of a subject. For example, a first assay may be used to process a first cell-free biological sample obtained or derived from the subject to generate a first dataset indicative of the pregnancy-related state; and a second assay different from the first assay may be used to process a second cell-free biological sample obtained or derived from the subject to generate a second dataset indicative of the pregnancy-related state. Any or all of the first dataset and the second dataset may then be analyzed to assess the pregnancy-related state of the subject. For example, a single diagnostic index or diagnosis score can be generated based on a combination of the first dataset and the second dataset. As another example, separate diagnostic indexes or diagnosis scores can be generated based on the first dataset and the second dataset.

The cell-free biological samples may be processed to identify a set of biomarker RNA transcripts that are indicative of a set of corresponding biomarker proteins (e.g., pregnancy-associated proteins corresponding to pregnancy-associated genomic loci or genes), pathways, and/or metabolites. For example, a given biomarker RNA transcript may be expected to be translated into a corresponding given biomarker protein or a gene regulator for a corresponding given biomarker protein. Therefore, identifying a presence or absence of the given biomarker RNA transcript in a biological sample may be indicative of a presence or absence of a corresponding biomarker protein. As another example, a given biomarker RNA transcript may be expected to correlate with a corresponding given pathway. Therefore, identifying a presence or absence of the given biomarker RNA transcript in a biological sample may be indicative of a presence or absence of the corresponding pathway activity. As another example, a given biomarker RNA transcript may be expected to correlate with a corresponding given biomarker metabolite. Therefore, identifying a presence or absence of the given biomarker RNA transcript in a biological sample may be indicative of a presence or absence of the corresponding biomarker metabolite. In some embodiments, the set of corresponding biomarker proteins, pathways, and/or metabolites comprises pregnancy-related state-associated proteins (e.g., corresponding to pregnancy-associated genomic loci or genes), pathways, and/or metabolites. In some embodiments, the set of corresponding biomarker proteins, pathways, and/or metabolites comprises placental proteins, pathways, and/or metabolites. For example, identifying a presence or absence of the PAPPA gene may be indicative of a presence or absence of the PAPPA protein analog.

The cell-free biological samples may be processed using a metabolomics assay. For example, a metabolomics assay can be used to identify a quantitative measure (e.g., indicative of a presence, absence, or relative amount) of each of a plurality of pregnancy-related state-associated metabolites in a cell-free biological sample of the subject. The metabolomics assay may be configured to process cell-free biological samples such as a blood sample or a urine sample (or derivatives thereof) of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. Assaying one or more metabolites of the cell-free biological sample may comprise isolating or extracting the metabolites from the cell-free biological sample. The metabolomics assay may be used to generate datasets indicative of the quantitative measure (e.g., indicative of a presence, absence, or relative amount) of each of a plurality of pregnancy-related state-associated metabolites in the cell-free biological sample of the subject.

The metabolomics assay may analyze a variety of metabolites in the cell-free biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The cell-free biological samples may be processed using a methylation-specific assay. For example, a methylation-specific assay can be used to identify a quantitative measure (e.g., indicative of a presence, absence, or relative amount) of methylation each of a plurality of pregnancy-related state-associated genomic loci in a cell-free biological sample of the subject. The methylation-specific assay may be configured to process cell-free biological samples such as a blood sample or a urine sample (or derivatives thereof) of the subject. A quantitative measure (e.g., indicative of a presence, absence, or relative amount) of methylation of pregnancy-related state-associated genomic loci in the cell-free biological sample may be indicative of one or more pregnancy-related states. The methylation-specific assay may be used to generate datasets indicative of the quantitative measure (e.g., indicative of a presence, absence, or relative amount) of methylation of each of a plurality of pregnancy-related state-associated genomic loci in the cell-free biological sample of the subject.

The methylation-specific assay may comprise, for example, one or more of a methylation-aware sequencing (e.g., using bisulfite treatment), pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The cell-free biological samples may be processed using a proteomics assay. For example, a proteomics assay can be used to identify a quantitative measure (e.g., indicative of a presence, absence, or relative amount) of each of a plurality of pregnancy-related state-associated proteins (e.g., corresponding to pregnancy-associated genomic loci or genes) or polypeptides in a cell-free biological sample of the subject. The proteomics assay may be configured to process cell-free biological samples such as a blood sample or a urine sample (or derivatives thereof) of the subject. A quantitative measure (e.g., indicative of a presence, absence, or relative amount) of pregnancy-related state-associated proteins (e.g., corresponding to pregnancy-associated genomic loci or genes) or polypeptides in the cell-free biological sample may be indicative of one or more pregnancy-related states. The proteins or polypeptides in the cell-free biological sample may be produced (e.g., as an end product, an intermediate product, or a byproduct) as a result of one or more biochemical pathways corresponding to pregnancy-related state-associated genes. Assaying one or more proteins or polypeptides of the cell-free biological sample may comprise isolating or extracting the proteins or polypeptides from the cell-free biological sample. The proteomics assay may be used to generate datasets indicative of the quantitative measure (e.g., indicative of a presence, absence, or relative amount) of each of a plurality of pregnancy-related state-associated proteins or polypeptides in the cell-free biological sample of the subject.

The proteomics assay may analyze a variety of proteins (e.g., pregnancy-associated proteins corresponding to pregnancy-associated genomic loci or genes) or polypeptides in the cell-free biological sample, such as proteins made under different cellular conditions (e.g., development, cellular differentiation, or cell cycle). The proteomics assay may comprise, for example, one or more of an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Kits

The present disclosure provides kits for identifying or monitoring a pregnancy-related state of a subject. A kit may comprise probes for identifying a quantitative measure (e.g., indicative of a presence, absence, or relative amount) of sequences at each of a plurality of pregnancy-related state-associated genomic loci in a cell-free biological sample of the subject. A quantitative measure (e.g., indicative of a presence, absence, or relative amount) of sequences at each of a plurality of pregnancy-related state-associated genomic loci in the cell-free biological sample may be indicative of one or more pregnancy-related states. The probes may be selective for the sequences at the plurality of pregnancy-related state-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of pregnancy-related state-associated genomic loci in a cell-free biological sample of the subject.

The probes in the kit may be selective for the sequences at the plurality of pregnancy-related state-associated genomic loci in the cell-free biological sample. The probes in the kit may be configured to selectively enrich nucleic acid (e.g., RNA or DNA) molecules corresponding to the plurality of pregnancy-related state-associated genomic loci. The probes in the kit may be nucleic acid primers. The probes in the kit may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct pregnancy-related state-associated genomic loci or genomic regions.

The instructions in the kit may comprise instructions to assay the cell-free biological sample using the probes that are selective for the sequences at the plurality of pregnancy-related state-associated genomic loci in the cell-free biological sample. These probes may be nucleic acid molecules (e.g., RNA or DNA) having sequence complementarity with nucleic acid sequences (e.g., RNA or DNA) from one or more of the plurality of pregnancy-related state-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of pregnancy-related state-associated genomic loci in the cell-free biological sample. A quantitative measure (e.g., indicative of a presence, absence, or relative amount) of sequences at each of a plurality of pregnancy-related state-associated genomic loci in the cell-free biological sample may be indicative of one or more pregnancy-related states.

The instructions in the kit may comprise instructions to measure and interpret assay readouts, which may be quantified at one or more of the plurality of pregnancy-related state-associated genomic loci to generate the datasets indicative of a quantitative measure (e.g., indicative of a presence, absence, or relative amount) of sequences at each of the plurality of pregnancy-related state-associated genomic loci in the cell-free biological sample. For example, quantification of array hybridization or polymerase chain reaction (PCR) corresponding to the plurality of pregnancy-related state-associated genomic loci may generate the datasets indicative of a quantitative measure (e.g., indicative of a presence, absence, or relative amount) of sequences at each of the plurality of pregnancy-related state-associated genomic loci in the cell-free biological sample. Assay readouts may comprise quantitative PCR (qPCR) values, digital PCR (dPCR) values, digital droplet PCR (ddPCR) values, fluorescence values, etc., or normalized values thereof.

A kit may comprise a metabolomics assay for identifying a quantitative measure (e.g., indicative of a presence, absence, or relative amount) of each of a plurality of pregnancy-related state-associated metabolites in a cell-free biological sample of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. A kit may comprise instructions for isolating or extracting the metabolites from the cell-free biological sample and/or for using the metabolomics assay to generate datasets indicative of the quantitative measure (e.g., indicative of a presence, absence, or relative amount) of each of a plurality of pregnancy-related state-associated metabolites in the cell-free biological sample of the subject.

### Trained Algorithms

After using one or more assays to process one or more cell-free biological samples derived from the subject to generate one or more datasets indicative of the pregnancy-related state or pregnancy-related complication, a trained algorithm may be used to process one or more of the datasets (e.g., at each of a plurality of pregnancy-related state-associated genomic loci) to determine the pregnancy-related state. For example, the trained algorithm may be used to determine quantitative measures of sequences at each of the plurality of pregnancy-related state-associated genomic loci in the cell-free biological samples. The trained algorithm may be configured to identify the pregnancy-related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a differential expression algorithm. The differential expression algorithm may comprise a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a pregnancy-related state. For example, an input variable may comprise a number of sequences corresponding to or aligning to each of the plurality of pregnancy-related state-associated genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's pregnancy-related state, and may comprise, for example, a therapeutic intervention, a duration of the therapeutic intervention, and/or a dosage of the therapeutic intervention suitable to treat a pregnancy-related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof. For example, such descriptive labels may provide a prognosis of the pregnancy-related state of the subject. As another example, such descriptive labels may provide a relative assessment of the pregnancy-related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a pregnancy-related state (e.g., pregnancy-related complication). For example, a binary classification of samples may assign an output value of “negative” or 0 if the sample indicates that the subject has less than a 50% probability of having a pregnancy-related state (e.g., pregnancy-related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a pregnancy-related state (e.g., pregnancy-related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a pregnancy-related state (e.g., pregnancy-related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a pregnancy-related state (e.g., pregnancy-related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a pregnancy-related state (e.g., pregnancy-related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}{30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a pregnancy-related state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the pregnancy-related state (e.g., training samples comprising cell-free biological samples and associated datasets and outputs obtained or derived from a plurality of subjects known to have the pregnancy-related state). Independent training samples may be associated with absence of the pregnancy-related state (e.g., training samples comprising cell-free biological samples and associated datasets and outputs obtained or derived from a plurality of subjects who are known to not have a previous diagnosis of the pregnancy-related state or who have received a negative test result for the pregnancy-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the pregnancy-related state and/or cell-free biological samples associated with absence of the pregnancy-related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the pregnancy-related state. In some embodiments, the cell-free biological sample is independent of samples used to train the trained algorithm.

The trained algorithm may be trained with a first number of independent training samples associated with presence of the pregnancy-related state and a second number of independent training samples associated with absence of the pregnancy-related state. The first number of independent training samples associated with presence of the pregnancy-related state may be no more than the second number of independent training samples associated with absence of the pregnancy-related state. The first number of independent training samples associated with presence of the pregnancy-related state may be equal to the second number of independent training samples associated with absence of the pregnancy-related state. The first number of independent training samples associated with presence of the pregnancy-related state may be greater than the second number of independent training samples associated with absence of the pregnancy-related state.

The trained algorithm may be configured to identify the pregnancy-related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The trained algorithm may be configured to identify the pregnancy-related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The trained algorithm may be configured to identify the pregnancy-related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The trained algorithm may be configured to identify the pregnancy-related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The trained algorithm may be configured to identify the pregnancy-related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The trained algorithm may be configured to identify the pregnancy-related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the pregnancy-related state.

The trained algorithm may be adjusted or tuned to improve one or more of the performance, accuracy, PPV, NPV, clinical sensitivity, clinical specificity, or AUC of identifying the pregnancy-related state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of pregnancy-related state-associated genomic loci may be identified as most influential or most important to be included for making high-quality classifications or identifications of pregnancy-related states (or subtypes of pregnancy-related states). The plurality of pregnancy-related state-associated genomic loci or a subset thereof may be ranked based on classification metrics indicative of each genomic locus's influence or importance toward making high-quality classifications or identifications of pregnancy-related states (or subtypes of pregnancy-related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring a Pregnancy-Related State

After using a trained algorithm to process the dataset, the pregnancy-related state or pregnancy-related complication may be identified or monitored in the subject. The identification may be based at least in part on quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites.

The pregnancy-related state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The pregnancy-related state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The pregnancy-related state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The pregnancy-related state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The pregnancy-related state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

In an aspect, the present disclosure provides a method for determining that a subject is at risk of pre-term birth, comprising assaying a cell-free biological sample derived from the subject to generate a dataset that is indicative of the pre-term birth risk at a specificity of at least 80%, and using a trained algorithm that is trained on samples independent of the cell-free biological sample to determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the pregnancy-related state is identified in a subject, a subtype of the pregnancy-related state (e.g., selected from among a plurality of subtypes of the pregnancy-related state) may further be identified. The subtype of the pregnancy-related state may be determined based at least in part on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites. For example, the subject may be identified as being at risk of a subtype of pre-term birth (e.g., selected from among a plurality of subtypes of pre-term birth). After identifying the subject as being at risk of a subtype of pre-term birth, a clinical intervention for the subject may be selected based at least in part on the subtype of pre-term birth for which the subject is identified as being at risk. In some embodiments, the clinical intervention is selected from a plurality of clinical interventions (e.g., clinically indicated for different subtypes of pre-term birth).

In some embodiments, the trained algorithm may determine that the subject is at risk of pre-term birth of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the pregnancy-related state, the subject may be optionally provided with a therapeutic intervention (e.g., prescribing an appropriate course of treatment to treat the pregnancy-related state of the subject). The therapeutic intervention may comprise a prescription of an effective dose of a drug, a further testing or evaluation of the pregnancy-related state, a further monitoring of the pregnancy-related state, an induction or inhibition of labor, or a combination thereof. If the subject is currently being treated for the pregnancy-related state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

The quantitative measures of sequence reads of the dataset at the panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites may be assessed over a duration of time to monitor a patient (e.g., subject who has pregnancy-related state or who is being treated for pregnancy-related state). In such cases, the quantitative measures of the dataset of the patient may change during the course of treatment. For example, the quantitative measures of the dataset of a patient with decreasing risk of the pregnancy-related state due to an effective treatment may shift toward the profile or distribution of a healthy subject (e.g., a subject without a pregnancy-related complication). Conversely, for example, the quantitative measures of the dataset of a patient with increasing risk of the pregnancy-related state due to an ineffective treatment may shift toward the profile or distribution of a subject with higher risk of the pregnancy-related state or a more advanced pregnancy-related state.

The pregnancy-related state of the subject may be monitored by monitoring a course of treatment for treating the pregnancy-related state of the subject. The monitoring may comprise assessing the pregnancy-related state of the subject at two or more time points. The assessing may be based at least on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined at each of the two or more time points.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of one or more clinical indications, such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a diagnosis of the pregnancy-related state of the subject. For example, if the pregnancy-related state was not detected in the subject at an earlier time point but was detected in the subject at a later time point, then the difference is indicative of a diagnosis of the pregnancy-related state of the subject. A clinical action or decision may be made based on this indication of diagnosis of the pregnancy-related state of the subject, such as, for example, prescribing anew therapeutic intervention for the subject. The clinical action or decision may comprise recommending the subject for a secondary clinical test to confirm the diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a prognosis of the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having an increased risk of the pregnancy-related state. For example, if the pregnancy-related state was detected in the subject both at an earlier time point and at a later time point, and if the difference is a negative difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased from the earlier time point to the later time point), then the difference may be indicative of the subject having an increased risk of the pregnancy-related state. A clinical action or decision may be made based on this indication of the increased risk of the pregnancy-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having a decreased risk of the pregnancy-related state. For example, if the pregnancy-related state was detected in the subject both at an earlier time point and at a later time point, and if the difference is a positive difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites decreased from the earlier time point to the later time point), then the difference may be indicative of the subject having a decreased risk of the pregnancy-related state. A clinical action or decision may be made based on this indication of the decreased risk of the pregnancy-related state (e.g., continuing or ending a current therapeutic intervention) for the subject. The clinical action or decision may comprise recommending the subject for a secondary clinical test to confirm the decreased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of an efficacy of the course of treatment for treating the pregnancy-related state of the subject. For example, if the pregnancy-related state was detected in the subject at an earlier time point but was not detected in the subject at a later time point, then the difference may be indicative of an efficacy of the course of treatment for treating the pregnancy-related state of the subject. A clinical action or decision may be made based on this indication of the efficacy of the course of treatment for treating the pregnancy-related state of the subject, e.g., continuing or ending a current therapeutic intervention for the subject. The clinical action or decision may comprise recommending the subject for a secondary clinical test to confirm the efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a non-efficacy of the course of treatment for treating the pregnancy-related state of the subject. For example, if the pregnancy-related state was detected in the subject both at an earlier time point and at a later time point, and if the difference is a negative or zero difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased or remained at a constant level from the earlier time point to the later time point), and if an efficacious treatment was indicated at an earlier time point, then the difference may be indicative of a non-efficacy of the course of treatment for treating the pregnancy-related state of the subject. A clinical action or decision may be made based on this indication of the non-efficacy of the course of treatment for treating the pregnancy-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In another aspect, the present disclosure provides a computer-implemented method for predicting a risk of pre-term birth of a subject, comprising: (a) receiving clinical health data of the subject, wherein the clinical health data comprises a plurality of quantitative or categorical measures of the subject; (b) using a trained algorithm to process the clinical health data of the subject to determine a risk score indicative of the risk of pre-term birth of the subject; and (c) electronically outputting a report indicative of the risk score indicative of the risk of pre-term birth of the subject.

In some embodiments, for example, the clinical health data comprises one or more quantitative measures of the subject, such as age, weight, height, body mass index (BMI), blood pressure, heart rate, glucose levels, number of previous pregnancies, and number of previous births. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and fetal screening results.

In some embodiments, the computer-implemented method for predicting a risk of pre-term birth of a subject is performed using a computer or mobile device application. For example, a subject can use a computer or mobile device application to input her own clinical health data, including quantitative and/or categorical measures. The computer or mobile device application can then use a trained algorithm to process the clinical health data to determine a risk score indicative of the risk of pre-term birth of the subject. The computer or mobile device application can then display a report indicative of the risk score indicative of the risk of pre-term birth of the subject.

In some embodiments, the risk score indicative of the risk of pre-term birth of the subject can be refined by performing one or more subsequent clinical tests for the subject. For example, the subject can be referred by a physician for one or more subsequent clinical tests (e.g., an ultrasound imaging or a blood test) based on the initial risk score. Next, the computer or mobile device application may process results from the one or more subsequent clinical tests using a trained algorithm to determine an updated risk score indicative of the risk of pre-term birth of the subject.

In some embodiments, the risk score comprises a likelihood of the subject having a pre-term birth within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

### Outputting a Report of the Pregnancy-Related State

After the pregnancy-related state is identified or an increased risk of the pregnancy-related state is monitored in the subject, a report may be electronically outputted that is indicative of (e.g., identifies or provides an indication of) the pregnancy-related state of the subject. The subject may not display a pregnancy-related state (e.g., is asymptomatic of the pregnancy-related state such as a pregnancy-related complication). The report may be presented on a graphical user interface (GUI) of an electronic device of a user. The user may be the subject, a caretaker, a physician, a nurse, or another health care worker.

The report may include one or more clinical indications such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject. The report may include one or more clinical actions or decisions made based on these one or more clinical indications. Such clinical actions or decisions may be directed to therapeutic interventions, induction or inhibition of labor, or further clinical assessment or testing of the pregnancy-related state of the subject.

For example, a clinical indication of a diagnosis of the pregnancy-related state of the subject may be accompanied with a clinical action of prescribing a new therapeutic intervention for the subject. As another example, a clinical indication of an increased risk of the pregnancy-related state of the subject may be accompanied with a clinical action of prescribing a new therapeutic intervention or switching therapeutic interventions (e.g., ending a current treatment and prescribing anew treatment) for the subject. As another example, a clinical indication of a decreased risk of the pregnancy-related state of the subject may be accompanied with a clinical action of continuing or ending a current therapeutic intervention for the subject. As another example, a clinical indication of an efficacy of the course of treatment for treating the pregnancy-related state of the subject may be accompanied with a clinical action of continuing or ending a current therapeutic intervention for the subject. As another example, a clinical indication of a non-efficacy of the course of treatment for treating the pregnancy-related state of the subject may be accompanied with a clinical action of ending a current therapeutic intervention and/or switching to (e.g., prescribing) a different new therapeutic intervention for the subject.

### Computer Systems

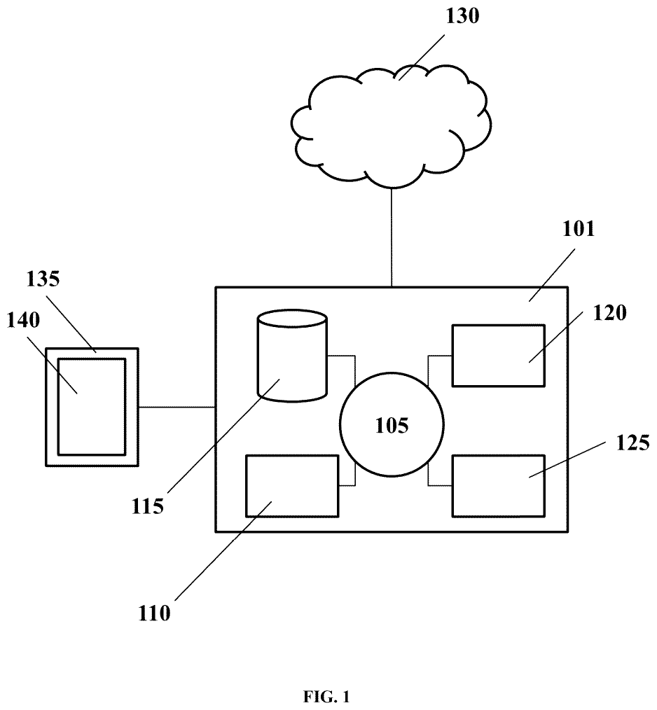

The present disclosure provides computer systems that are programmed to implement methods of the disclosure. FIG. 1 shows a computer system 101 that is programmed or otherwise configured to, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

The computer system 101 can regulate various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet.

The network 130 in some cases is a telecommunication and/or data network. The network 130 can include one or more computer servers, which can enable distributed computing, such as cloud computing. For example, one or more computer servers may enable cloud computing over the network 130 (“the cloud”) to perform various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. Such cloud computing may be provided by cloud computing platforms such as, for example, Amazon Web Services (AWS), Microsoft Azure, Google Cloud Platform, and IBM cloud. The network 130, in some cases with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, (i) a visual display indicative of training and testing of a trained algorithm, (ii) a visual display of data indicative of a pregnancy-related state of a subject, (iii) a quantitative measure of a pregnancy-related state of a subject, (iv) an identification of a subject as having a pregnancy-related state, or (v) an electronic report indicative of the pregnancy-related state of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

### EXAMPLES

### Example 1: Early Prediction of Spontaneous Preterm Birth and Late Miscarriages in a High-Risk Population Using Molecular Sub-Typing with cfRNA Profiling

Using systems and methods of the present disclosure, early molecular markers of preterm birth (PTB) and stillbirth were identified in maternal blood samples from a population at increased risk of PTB due to clinical history.

The study design was performed as follows. Blood samples from 229 women were collected between weeks 12-24 of gestation (IQR 18.9-20.9) and before onset of labor. Samples were collected across 4 independent sites in the UK, and 51% of the cohort were considered to be at high-risk, defined by at least one of prior sPTB (16-36), previous cervical surgery, or a cervical length of less than 25 mm. 80% (n=183) delivered at term and 20% (n=46) had a sPTB (GA of less than 35 weeks). 30% of the sPTB delivered at a GA of less than 25 weeks. 80% (n=183) delivered at term and 20% (n=46) had a sPTB (GA of less than 35 weeks). 30% of the sPTB delivered at a GA of less than 25 weeks. All samples were processed using a unified experimental and computational NGS pipeline for cell-free (cfRNA) sequencing. Twin pregnancies and cases of preeclampsia were excluded.

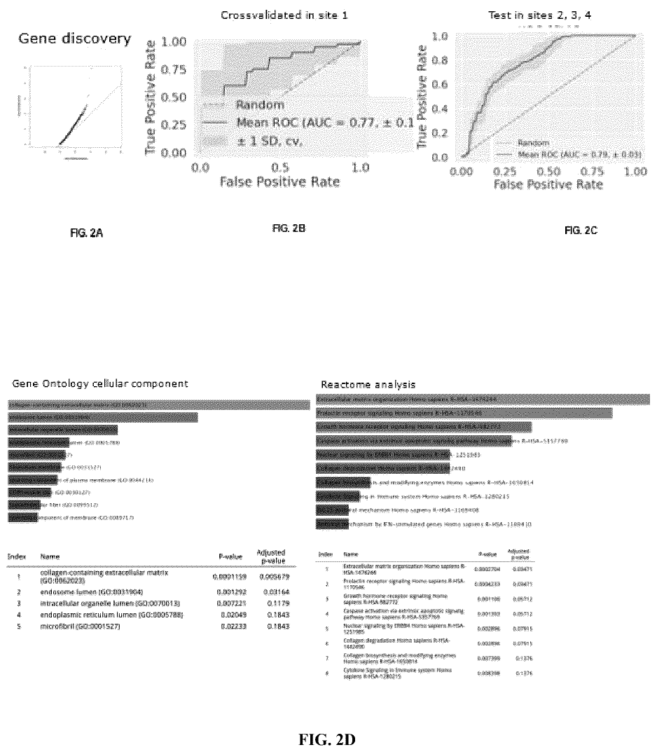

cfRNA profiling was performed of plasma obtained from all 229 maternal blood samples. 41 genes (COL3A1, HSD17B1, GPC4, CDR1-AS, COL5A1, COL6A1, COL1A1, EFHD1, LENG8, DCN, CYB5R2, ELN, ANTXR1, CH507-24F1.2, EIF4A1, ABI3BP, LAPTMS, SLC38A10, CCDC80, CIS, VPS45, COL1A2, C7, FN1, COL14A1, ARPC4-TTLL3, LINC01002, PSG4, TMTC2, STAG3L5P, AMT, TAP1, CSH1, MMP2, PLXNA3, LGMNP1, LUM, MYO18B, DAPK2, GCM1L, GALS14, FNDC10, FBN2, CAPN13, TNFRSF25, MYH11, POGLUT1, GH2, DNAH1, DES, NUP210) were identified as being associated with an increased risk of sPTB (FDR<0.1). Based on these transcripts, a logistic regression classifier model was developed to predict PTB, obtaining an AUC=0.72, as shown in FIGS. 2A-2C. The model was validated through leave-one-out cross-validation (LOOCV). Additional insight in the pathophysiology of PTB are presented in FIG. 2D. Pathway analysis for the transcripts associated with sPTB revealed an enrichment of genes related to collagen-containing and extracellular matrix in those individuals who ultimately had a sPTB at a GA of less than 35 weeks.

Additional analysis for extremely late miscarriage/early pre-term birth (GA of less than 25 weeks at delivery) was performed on 14 cases and 166 at term controls. A logistic regression classifier was developed based on 11 genes (AC011043.1, IGFBP2, SH3GL3, AMT, GTF2IP4, GYPB, PAPPA, CH17-472G23.2, OMA1, ACADSB, ACER3) to predict risk of extremely late miscarriage/early pre-term birth with an enhanced performance of AUC=0.76 in LOOCV. While enrichment for were associated with sPTB at a GA of less than 25 weeks.

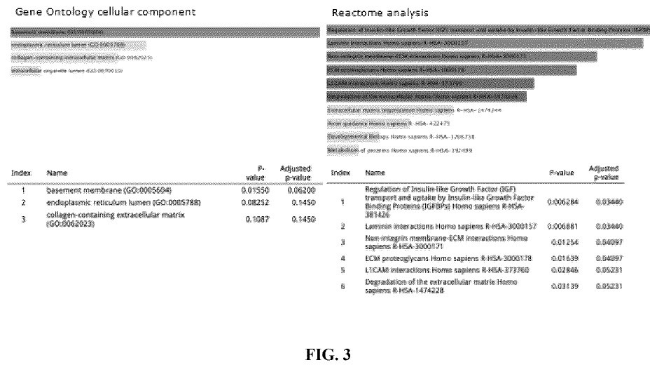

Pathway analysis of the 6-transcript set revealed an enrichment of a set of genes related to basement membrane and endoplasmic reticulum lumen, and genes in insulin-like growth factor transport and uptake and amino acid metabolism pathways in samples from maternal subjects that deliver before a GA of 25 weeks, providing additional insight in the pathophysiology of extremely late miscarriage/early pre-term birth as presented in FIG. 3.

The analysis of cfRNA in maternal plasma provided a noninvasive window to maternal-fetal health in pregnancies at increased risk of pregnancy complications. Our data showed that elevated expression of a subset of transcripts potentially involved in at least two molecular subtype of preterm birth and two different underlining mechanisms. In a first molecular subtype, a collagen-containing extracellular matrix pathway may be associated with cervical remodeling associated with shorter cervix, and cervix insufficiency may form a basis for underling biology for high risk PTB delivery. In a second molecular subtype, for endoplasmic reticulum lumen pathway, there may be an association of endoplasmic reticulum stress induced by oxidative stress in decidual cells with a possible mechanism of early pregnancy loss. Further, a basement membrane pathway may indicate the premature placental membrane separation from uterus during miscarriage.

Based on these results of the molecular sub-typing for PTB, specific treatments may be selected and administered to maternal subjects based on a particular molecular subtype, to modulate the outcome of pre-term birth. For example, collagen modulating therapeutics or cervical cerclage can be applied to stabilize the cervix. The late miscarriage cases may be prevented by administering therapeutics to reduce oxidative stress and/or modulating expression levels of proteins related to endoplasmic reticulum stress.

### Example 2: Early Prediction of Preeclampsia Using Molecular Sub-Typing with cfRNA Profiling

Using systems and methods of the present disclosure, early molecular markers of preeclampsia (PE) are identified in maternal blood samples from a population at increased risk of preeclampsia (PE) due to clinical history.

Further, a cohort of subjects includes a set of control subjects with delivery after 37 weeks of gestational age. Some control subjects are classified as healthy controls, and some control subjects have a history of chronic hypertension without preeclampsia. A set of case subjects are diagnosed with preeclampsia with delivery before 37 weeks of gestational age. A set of case subjects are diagnosed with de novo preeclampsia, and a subset of case subjects have preeclampsia with a history of chronic hypertension.

Differential expression analysis of the cohort data set is performed as follows. Biomarker discovery is performed to identify early diagnostic markers of preeclampsia using cell-free RNA. In order to estimate the effect of chronic hypertension, two separate differential expression analyses are performed to estimate the effect of chronic hypertension. A first analysis is performed on a set of preeclampsia cases and a set of healthy controls; further, a second analysis is performed, in which a set of control subjects with chronic hypertension is added, thereby totaling a larger number of control subjects.

A set of top differentially expressed genes for PE in the cohort is identified for both comparisons including chronic hypertension and excluding chronic hypertension. The top genes from both analyses are observed to overlap, which is indicative of a signal associated with preeclampsia, and not chronic hypertension.

Additional analysis of highly significant genes associated with higher risk of PE indicates at least two separate pathways with different underlying biology. In a first molecular subtype, the enrichment of low expressed placenta-specific genes like PAPA2 and FABP1 indicates significant changes in early placentation, which may be associated with preeclampsia. Also, a high dose of aspirin administered at early pregnancy before 13 weeks may reduce the risk of extremely early onset of PE (<32 weeks) but may not reduce the PE developed later. In a second molecular subtype, the pathways associated with keratinocyte endothelium may be associated with vascular inflammation, endothelial dysfunction, and arterial hypertension. Moreover, the skin holds a complex capillary counter current system which controls body temperature, skin perfusion, and apparently systemic blood pressure. Therefore, the case of once of preeclampsia can be associated with underlying biology of mother's skin, capillary, and or arterial dysfunction.

Based on these results of molecular sub-typing for PE, specific treatments may be selected and administered to maternal subjects to modulate the outcome or risk of developing preeclampsia. For example, subjects with a molecular subtype of placentation may be treated with compounds similar to aspirin, which is associated with regulation of cyclooxygenase pathways or pathways responsible for modulating the vasodilatory mediators and inhibiting vascular remodeling, platelet aggregation, and platelet adhesion. As another example, subjects with a molecular subtype of PE associated with keratinocyte endothelium pathways may be treated with blood pressure management compounds or compounds targeted to a mechanism of proton pump inhibitors (PPI). Blocking PPI may lead to decreased sFlt-1 and soluble endoglin (sENG) secretion and endothelial dysfunction, dilation of blood vessels, decreased BP, and antioxidant and anti-inflammatory properties. Use of Esomeprazole, another proton pump inhibitor that is also used for gastric reflux, may be evaluated in phase II clinical studies to treat early onset PE (PIE Trail) in maternal subjects.

### Example 3: Early Prediction of Gestational Diabetes Mellitus (GDM) Using Molecular Sub-Typing with cfRNA Profiling

Using systems and methods of the present disclosure, early molecular markers of diabetes mellitus (GDM) are identified in maternal blood samples from a population at increased risk of GDM due to clinical history.

Further, a cohort of subjects includes a set of control subjects. Some control subjects are classified as healthy controls with negative Oral Glucose Tolerance Test (OGTT) test. A first set of case subjects are diagnosed with gestational GDM based on OGTT test, a second set of case subjects are diagnosed with chronic Type 2 diabetes, and a third set of case subjects have impaired glucose status.

Differential expression analysis of the cohort data set is performed as follows. Biomarker discovery is performed to identify early diagnostic markers of GDM using cell-free RNA. In order to estimate the effect of chronic Type 2 diabetes, two separate differential expression analyses are performed to estimate the effect. A first analysis was performed on a set of gestational GDM cases and a set of healthy controls; further, a second analysis is performed, in which a set of control subjects with chronic Type 2 diabetes are added, thereby totaling a larger number of control subjects.

A set of top differentially expressed genes for GDM in the cohort is identified for both comparisons including chronic Type 2 diabetes and excluding chronic Type 2 diabetes. The top genes from both analyses are observed to overlap, which is indicative of a signal associated with GDM, and not chronic Type 2 diabetes.

Additional analysis of highly significant genes associated with higher risk of GDM indicates at least three separate pathways with different underlying biology. In a first molecular subtype, the enrichment of low expressed placenta-specific genes (PDK4, CSH1, and PLAC4) indicates significant changes as placenta deterioration, placenta insufficiency, placenta failure, placenta dysfunction, premature ageing, calcification and impaired placenta function with gestational diabetes. In a second molecular subtype, one of genes TBCEL, tubulin-specific chaperone cofactor E-like, may be associated with mediated hyperglycemic memory, which may be common for type 1 and type 2 diabetes. In a third molecular subtype, FBXO7 gene is involved in adaptive immune system and antigen-specific immune response efficiently pathways. GDM is characterized not only by increased insulin resistance and glucose intolerance, but also by a state of low-grade systemic inflammation and dysregulation of the immune system which induces an imbalance between type 1 and 2 T-helper cells.

Based on these results of molecular sub-typing for GDM, specific treatments may be selected and administered to maternal subjects to modulate the outcome or risk of developing GDM. For example, subjects with a molecular subtype indicative of significant changes in placenta disfunction or deterioration may be treated with potential candidate drug targets—effectors to improve uteroplacental blood flow, anti-oxidants, heme oxygenase induction, inhibition of HIF, induction of cholesterol synthesis pathways, increasing insulin-like growth factor II availability. As another example, for subjects with second molecular subtype associated with mediated hyperglycemic memory, an early aggressive treatment of this glucose imbalance may be administered to subjects with diabetes. Hyperglycemia may be accompanied by the formation of advanced glycation end products (AGEs). Another therapeutic approach may be to attempt to reduce AGE formation, receptor of AGE (RAGE) expression, and oxidative stress generation. Different drugs may be administered to block AGE formation, such as metformin and pioglitazone. ACE inhibitors and AT-1 blockers are compounds used to control blood pressure; however, they are also capable of reducing AGEs formation. Telmisartan downregulates RAGE mRNA levels and subsequently inhibits superoxide generation, whereas gliclazide may be useful in abolishing the “memory”. In addition, GLP1 receptor agonists may be administered to decrease inflammation, postprandial hyperlipidemia, and coagulation, resulting in a beneficial effect on atherothrombosis. Aldose reductase inhibitors like Epalrestat may be administered to protect against diabetic peripheral neuropathy by alleviating oxidative stress and inhibiting polyol pathway. As another example, for subjects with a third molecular subtype involved in adaptive immune system and antigen-specific pathways, pregnancy is a significant metabolic and immune challenge; further, GDM superimposes an enhanced degree of low-grade systemic inflammation and uncompensated insulin resistance, which may be further linked to a dysregulation of the underlying immune response, which may be common for Type 1 and Type 2. Immunomodulators may be administered to treat diabetes, such as: Azathioprine, Mycophenolate mofeti, Otelixizumab, Teplizumab (which may minimize cytokine release and prevent the progressive destruction of β-cells).

### Example 4: Predictive RNA Profiles for Early and Very Early Spontaneous Preterm Birth in a High-Risk Population and Pathway Analysis Using the Reactome Database

Using systems and methods of the present disclosure, early molecular markers of preterm birth (PTB) and very early spontaneous preterm birth (sPTB) were identified in maternal blood samples from a population to clinical history of high risk.

High-risk pregnancies were defined by at least one of prior sPTB or late miscarriage (between 12 to 37 weeks of gestation), previous destructive cervical surgery, or incidental finding of a cervical length <25 mm on transvaginal ultrasound scan. Women with no risk factors for sPTB and otherwise well at the time of enrollment were recruited as low-risk controls from routine antenatal or ultrasonography clinics.

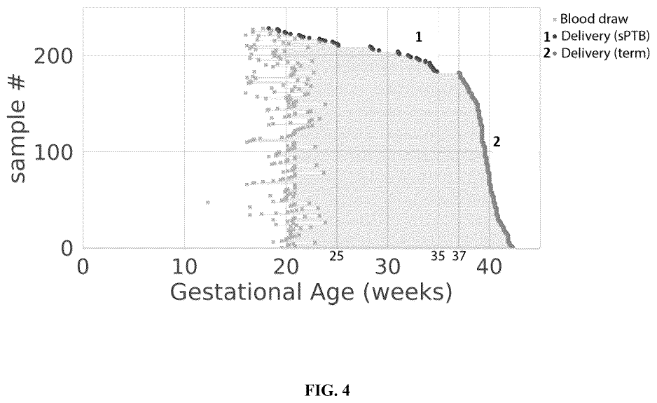

Blood samples were collected between 12 and 24 weeks of gestation (242 blood samples, one sample per pregnancy) from women with singleton pregnancies recruited from four tertiary antenatal clinics. For sPTB cases, samples were collected on average 9.4 weeks before delivery. Distribution of 242 collected samples with gestational age at blood sample collection, and gestational age at delivery are shown in FIG. 4. Out of 242 pregnancies, 194 delivered at term (≥37 0/7 GA), and 48 spontaneously delivered preterm before 35 weeks gestation (early preterm, <35 0/7). A subset of 16 of the pregnancies delivered before 25 weeks gestation (very early preterm, <25 0/7).

To identify candidate genes that can be predictive of risk of early sPTB (<35 0/7), differential expression analyses were performed between all early deliveries (<35 0/7) and controls (≥37 0/7). Results were validated using Leave-One-Out Cross-Validation (LOOCV), which resulted in a list of 25 differentially expressed genes listed in Table 1 that were used to build a logistic regression classifier to predict risk of preterm birth.

TABLE 1
Early sPTB differentially expressed genes
identified by LOOCV with corresponding
identification frequency across the folds.

| Index | Gene | % Folds identified |
|---|---|---|
| 1 | COL14A1 | 100.0 |
| 2 | GCM1 | 100.0 |
| 3 | CH507-24F1.2 | 100.0 |
| 4 | GH2 | 100.0 |
| 5 | CYB5R2 | 100.0 |
| 6 | CAPN13 | 98.8 |
| 7 | STAG3L5P | 100.0 |
| 8 | GPC4 | 99.2 |
| 9 | LGALS14 | 100.0 |
| 10 | ELN | 98.3 |
| 11 | NPR2 | 100.0 |
| 12 | TNFRSF25 | 100.0 |
| 13 | HRH1 | 98.8 |
| 14 | ZNF404 | 100.0 |
| 15 | SIGLEC8 | 91.3 |
| 16 | MTHFD2P7 | 93.3 |
| 17 | PAGE4 | 50.4 |
| 18 | RGPD5 | 4.2 |
| 19 | ZNF812P | 6.7 |
| 20 | MTCO3P12 | 2.9 |
| 21 | AL773572.7 | 2.1 |
| 22 | LINC00969 | 0.4 |
| 23 | OLFML3 | 0.8 |
| 24 | FLG2 | 0.4 |
| 25 | AP000580.1 | 0.4 |

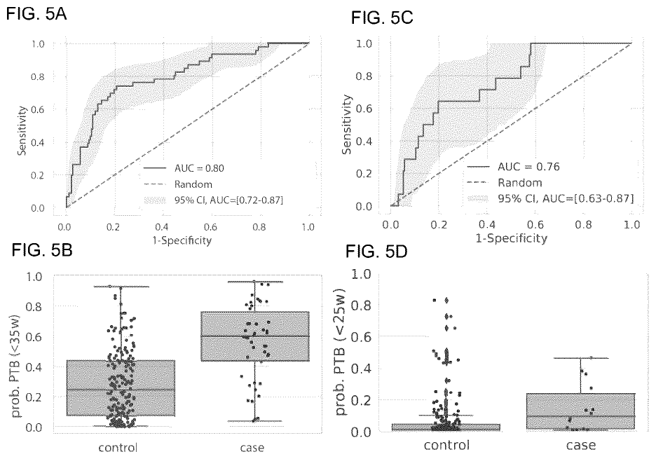

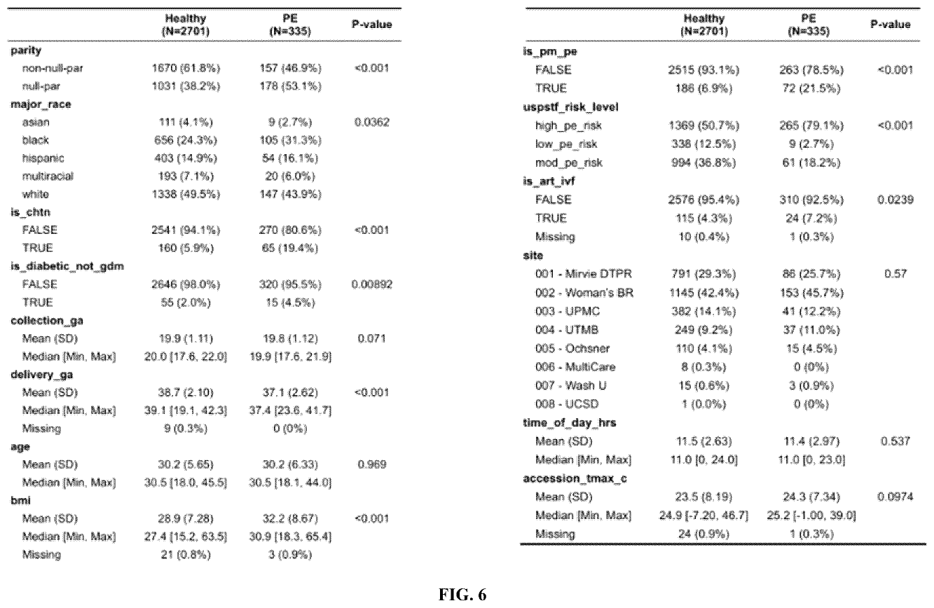

The model achieved a validated LOOCV performance area under the curve (AUC) of 0.80 (95% CI 0.72-0.87) with sensitivity=0.76 and specificity=0.72 (N=46 early sPTB cases and N=183 at-term controls) shown in FIG. 5A. The model also scored each sample with a risk probability of preterm delivery shown in FIG. 5B.

The same approach was used to identify molecular markers specific to very early sPTB (<25 weeks). Differential expression analyses were performed between 16 very early PTB cases and 226 controls, and the results were validated using LOOCV. A list of 65 differentially expressed genes was generated (Table 2), that were used to build a regularized logistic regression classifier to predict very early sPTB in cross-validation.

TABLE 2
Very early sPTB differentially expressed genes
identified by LOOCV with corresponding
identification frequency across the folds.

| Index | Gene | % Folds Identified |
|---|---|---|
| 1 | AC011043.1 | 96.3 |
| 2 | SCN3A | 93.8 |
| 3 | SMAD5 | 88.0 |
| 4 | PLAC4 | 81.8 |
| 5 | TUBB2A | 74.4 |
| 6 | SEL1L3 | 74.0 |
| 7 | MCM6 | 69.0 |
| 8 | CUX2 | 68.6 |
| 9 | PPL | 60.7 |
| 10 | PRKG2 | 57.9 |
| 11 | CATSPERB | 55.8 |
| 12 | ACE | 54.1 |
| 13 | GTF2IP4 | 46.3 |
| 14 | KRT5 | 43.0 |
| 15 | AGPAT4 | 33.5 |
| 16 | ZCCHC7 | 33.1 |
| 17 | CYP19A1 | 28.9 |
| 18 | RGPD8 | 26.0 |
| 19 | TMEM70 | 26.0 |
| 20 | MTRNR2L1 | 26.0 |
| 21 | DDX11L10 | 21.5 |
| 22 | OLFM1 | 18.2 |
| 23 | Clorf21 | 9.5 |
| 24 | RPH3AL | 9.5 |
| 25 | LURAP1L | 7.0 |
| 26 | SPATA7 | 5.8 |
| 27 | CH17-472G23.2 | 4.5 |
| 28 | MXRA7 | 3.7 |
| 29 | PARD3B | 3.3 |
| 30 | UPK1A-AS1 | 3.3 |
| 31 | MT-ND6 | 2.9 |
| 32 | MKRN9P | 2.5 |
| 33 | FUT8 | 2.5 |
| 34 | ZNF528 | 2.5 |
| 35 | H3F3BP1 | 2.1 |
| 36 | FAM83D | 2.1 |
| 37 | AP003068.18 | 2.1 |
| 38 | CH17-431G21.1 | 1.7 |
| 39 | SH3GL3 | 1.7 |
| 40 | GSTM1 | 1.7 |
| 41 | CSH1 | 1.2 |
| 42 | GALNT12 | 1.2 |
| 43 | FCGR2A | 0.8 |
| 44 | EPS8L1 | 0.8 |
| 45 | RP11-514O12.4 | 0.8 |
| 46 | ZNF117 | 0.8 |
| 47 | PODXL | 0.8 |
| 48 | DHRSX | 0.8 |
| 49 | CD79A | 0.4 |
| 50 | CMA1 | 0.4 |
| 51 | XKR8 | 0.4 |
| 52 | FAM171A1 | 0.4 |
| 53 | DHCR7 | 0.4 |
| 54 | GPX1P1 | 0.4 |
| 55 | PHGDH | 0.4 |
| 56 | PAX8-AS1 | 0.4 |
| 57 | CNFN | 0.4 |
| 58 | PRPSAP1 | 0.4 |
| 59 | C5orf34 | 0.4 |
| 60 | LYSMD4 | 0.4 |
| 61 | IGFBP5 | 0.4 |
| 62 | TRBV20-1 | 0.4 |
| 63 | IGLC3 | 0.4 |
| 64 | KCNG1 | 0.4 |
| 65 | PPP2CB | 0.4 |

The model achieved a validated LOOCV performance of AUC=0.74 (95% CI 0.64-0.83). Several genes were found to be related genes involved in preeclampsia toxemia (PET). To reduce crosstalk in the cfRNA signature across the two complications, modeling was performed by excluding all PET samples and samples with low quality sequencing metrics, given the reduction to 14 cases of very early preterm (<25 0/7). The model exhibited improved LOOCV performance with AUC=0.76 (95% CI 0.63-0.87) [sensitivity=0.64, specificity=0.80] for 14 very early sPTB cases (<25 0/7) and 193 samples that delivered at or after 25 weeks (>25 0/7), as shown in FIG. 5C. The model was based on a set of 39 differentially expressed genes listed in Table 3, from which a core set of three genes (AC011043.1, IGFBP2, and SH3GL3) was identified in >9500 of cross-validation folds, and 13 genes overlapped with the differentially expressed genes discovered when training with the PET samples. The model probabilities showed a significant difference between cases and controls, although a longer tail of high sPTB probabilities was observed for a subset of control samples, as shown in FIG. 51D.

TABLE 3
Very early sPTB differentially expressed genes identified
by LOOCV with corresponding identification frequency
across the folds after exclusion of PET samples.

| Index | Gene | % Folds Identified |
|---|---|---|
| 1 | AC011043.1 | 100.0 |
| 2 | IGFBP2 | 99.5 |
| 3 | SH3GL3 | 95.7 |
| 4 | AMT | 89.4 |
| 5 | GTF2IP4 | 85.0 |
| 6 | GYPB | 81.6 |
| 7 | PAPPA | 56.5 |
| 8 | CH17-472G23.2 | 33.3 |
| 9 | OMA1 | 26.6 |
| 10 | ACADSB | 23.7 |
| 11 | ACER3 | 20.8 |
| 12 | MXRA7 | 9.2 |
| 13 | PCTP | 6.8 |
| 14 | TUBB2A | 6.8 |
| 15 | RNY4 | 6.3 |
| 16 | FAM171A1 | 6.3 |
| 17 | ILDR2 | 4.3 |
| 18 | NDST3 | 3.9 |
| 19 | MISP3 | 2.9 |
| 20 | GSTM2 | 2.9 |
| 21 | PHGDH | 2.4 |
| 22 | Clorf21 | 2.4 |
| 23 | TMEM140 | 1.9 |
| 24 | FAM111B | 1.9 |
| 25 | DDX11L10 | 1.4 |
| 26 | PPBP | 1.4 |
| 27 | MT-TV | 1.0 |
| 28 | MOB3C | 1.0 |
| 29 | RUNX1T1 | 1.0 |
| 30 | NEDD8-MDP1 | 1.0 |
| 31 | AMD1 | 1.0 |
| 32 | FAM83D | 1.0 |
| 33 | BMS1P10 | 0.5 |
| 34 | RAI14 | 0.5 |
| 35 | RNF144A | 0.5 |
| 36 | VPS37B | 0.5 |
| 37 | CATSPERB | 0.5 |
| 38 | FUT8 | 0.5 |
| 39 | TCEAL8 | 0.5 |

The analysis of biological pathways driving sPTB predictive genes was performed using the Reactome database and top two pathways for each preterm models listed in Table 4.

TABLE 4
Pathway analysis of sPTB differentially expressed genes.
Pathway analysis for genes discovered in the early sPTB predictor
(<35 0/7) and the very early sPTB predictor (<25 0/7).
Early sPTB predictor (<35 0/7)

| Term | P-value | Adjusted P-value |
|---|---|---|
| Extracellular matrix organization | 5.12E−03 | 0.098 |
| (R-HSA-1474244) | | |
| Degradation of the extracellular | 7.71E−03 | 0.098 |
| matrix (R-HSA-1474228) | | |

Very early sPTB predictor (<25 0/7)

| Term | P-value | Adjusted P-value |
|---|---|---|
| Regulation of Insulin-like Growth | 7.60E−04 | 0.043 |
| Factor (IGF) transport and | | |
| uptake by IGFBP (R-HSA-381426) | | |
| Metabolism of amino acids and | 4.01E−03 | 0.112 |
| derivatives (R-HSA-71291) | | |

The early preterm birth model (<35 0/7) was enriched for genes involved in extracellular matrix (ECM) degradation and remodeling. The data were in agreement with the observation that mediators of cell-to-cell adhesion such as undulin (COL14A1) and elastin (ELN) are among the top genes in the model. Early detection of FECM pathways from a blood draw may serve to identify individuals at risk for premature cervical remodeling. Such a screening test may be implemented at a similar window as ultrasound to measure cervical length in women at high-risk.

By contrast, a similar analysis performed on the genes obtained in the very early preterm birth model (<25 0/7 weeks) revealed that pathways related to insulin-like growth factor transport and amino acid metabolism pathways were observed to be differentially expressed in very early sPTB. Insulin binding proteins are highly expressed by the fetus and in the decidua basalis, and are key regulators of the bioavailability of IGF-1 and hence fetal growth. For instance, IGFBP1 is associated with intrauterine growth restriction and impaired placentation, and is raised in cord blood from extremely preterm infants. The detection of insulin-growth factor cfRNA, as a predominant signal in pregnancies that result in very early sPTB is both plausible and potentially informative in terms of downstream events.

### Example 5: Prospective Cohort Subjects for Preeclampsia Case Studies and cfRNA NGS Data Correction

Using systems and methods of the present disclosure, a prospective, observational study was performed of a cell-free RNA platform utilizing direct-to-participant recruitment via targeted social media from July 2020 to April 2022. The IRB-approved study was open to subjects of ages 18 to 45 with a singleton pregnancy in the United States. Participants signed informed consent, provided record release forms, completed a short questionnaire, and submitted blood samples through mobile phlebotomy scheduled via a web-based platform.

Participants submitted blood samples between 17 and 22 weeks of gestation age. Medical records were received for over 85% of participants. The cohort is geographically and ethnically diverse, representing 1,220 zip codes across 30 states. All samples were processed using a unified experimental and computational NGS pipeline for cell-free RNA (cfRNA) sequencing.

3,036 samples had complete medical records and passed cfRNA assay quality metrics. FIG. 6 shows the demographic and clinical data metrics for the preeclampsia observational studies, including 2,701 healthy participants and 335 participants diagnosed with preeclampsia.

FIG. 6 shows the distribution of demographic and clinical factors within this cohort associated with risk of developing preeclampsia, which were collected based on U.S. Preventive Services Task Force (USPSTF) guidelines and recommendations. Various preeclampsia risk factors were recorded for this cohort, including: parity; race; chronic hypertension (chtn); diabetic status excluding gestational diabetes (diabetic not_gdm); mothers age; body mass index (bmi); prior preeclampsia diagnosis (pm_pe); U.S. Preventive Services Taskforce (USPSTF) risk level (www.uspreventiveservicestaskforce.org/uspstf/recommendation/preeclampsia-screening); and artificial in vitro fertilization (IVF).

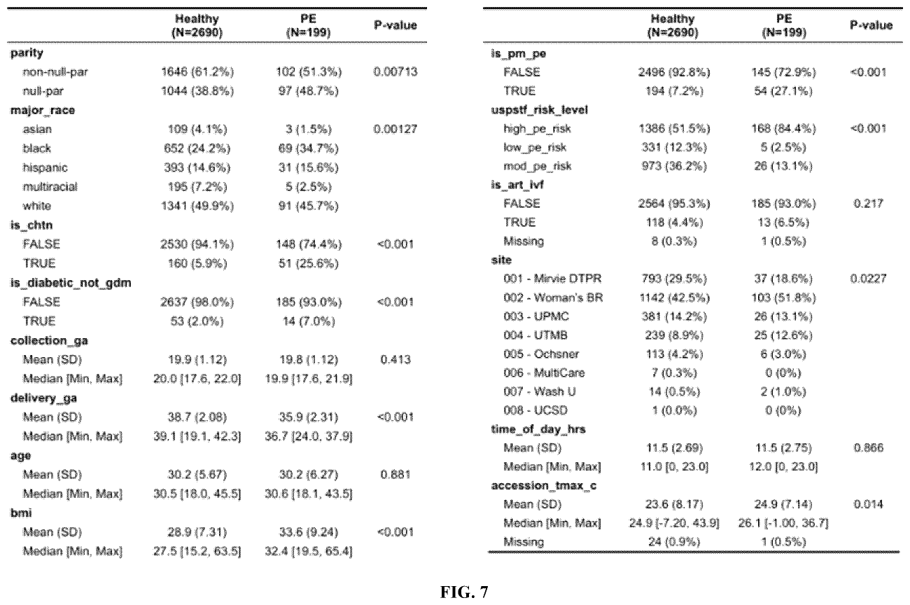

To define the different subtypes of preeclampsia, different cutoffs for medically-indicated deliveries (delivery_GA) for preeclampsia-diagnosed patients were used. FIG. 7 shows the demographic of the same cohort of 2,889 samples for preeclampsia with delivery at less than 38 weeks, including 2,690 healthy participants and 199 participants who were diagnosed with preeclampsia and delivered before 38 weeks of gestational age.

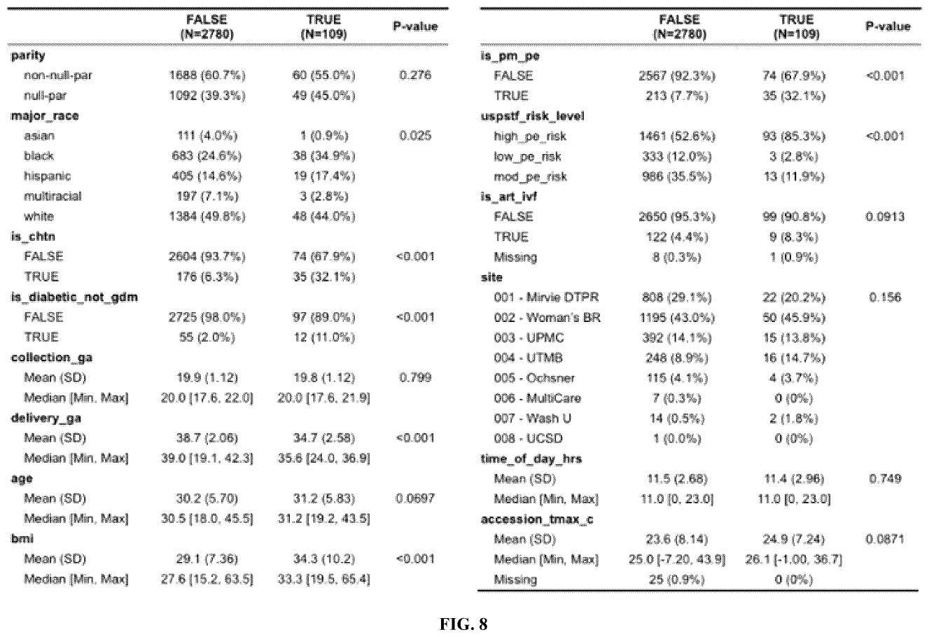

FIG. 8 shows the demographic of the same cohort of 2,889 samples for preeclampsia with delivery at less than 37 weeks, including 2,780 healthy participants and 109 participants who were diagnosed with preeclampsia and delivered before 37 weeks of gestational age.

Cell-free RNA (cfRNA) level measures in plasma quantified by NGS techniques can be affected by systematic variation due to the technical processing of samples, which may compromise the accuracy of the measurement process and contribute to bias the estimate of the association under investigation. The quantification of the contribution of the systematic source of variation is challenging in datasets characterized by hundreds of thousands of features.

Several sources of systematic variations in the 3,036-sample cfRNA data set were identified, and several statistical correction methodologies were applied. These correction techniques included several methodologies: 1) residuals from multivariate linear regression were used to correct the data residuals, 2) a ComBat method was performed based on an empirical Bayes approach that can correct only for one covariate at the time; 3) and surrogate variables analysis (SVA) was developed to remove pre-identified sources of variability but also unknown sources of variability. The correction methodology using residuals from multivariate linear regression to correct the data for the effects of unwanted covariates demonstrated better correction on a complete dataset as compared to the other two techniques.

Various sources of variation were identified by analysis of the 3,036-sample cfDNA data set, and were successfully corrected by performing various methodologies. First, the technical variations attributed to NGS-specific methodology such as: depth of sequencing per sample; batch effects for individual process operations; or various raw materials were identified and corrected.

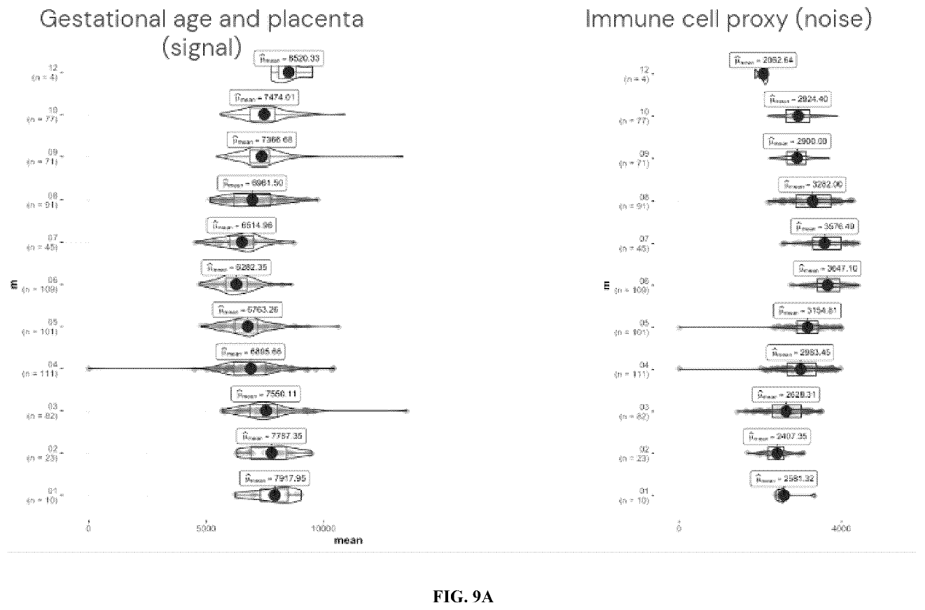

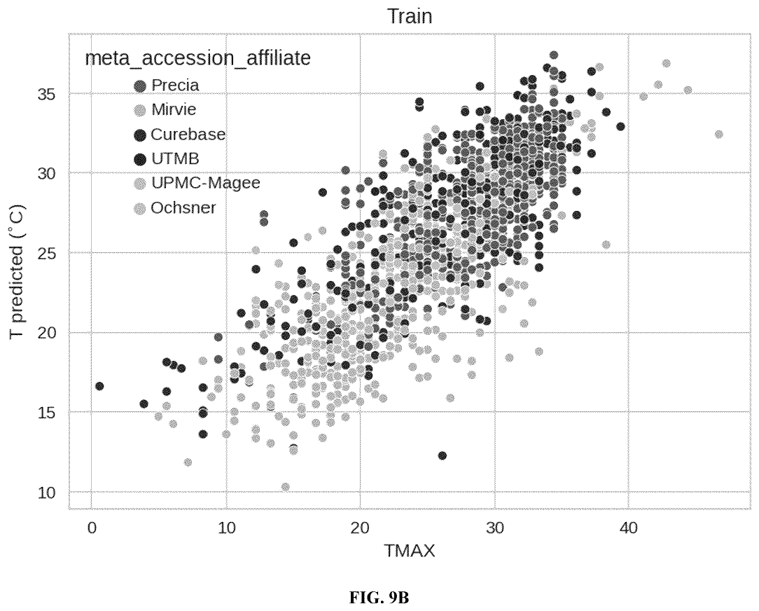

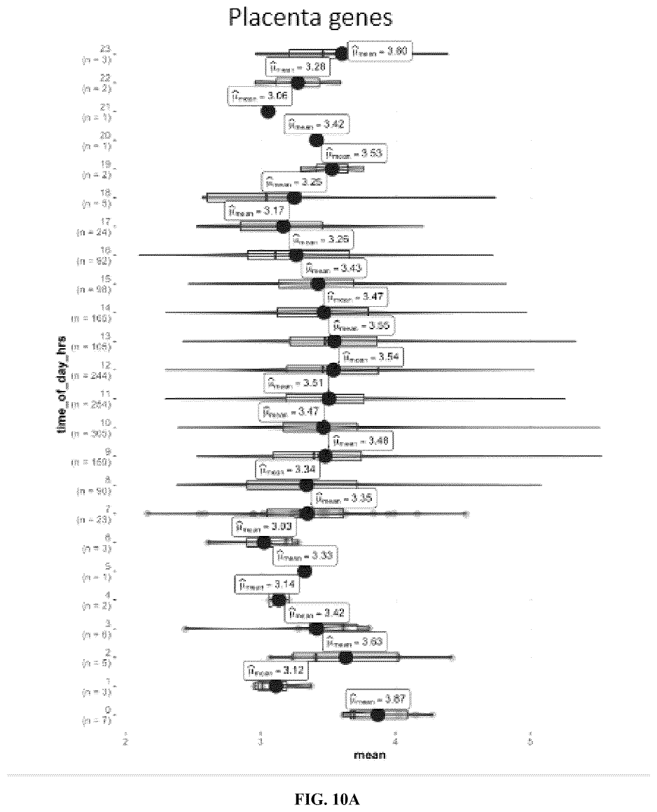

Further, two external sources of variation were identified related to time of blood collection, and corrected by multivariate linear regression to correct the data residuals. FIG. 9A shows an example of systematic variance in NGS data associated with seasonal changes for gestational- and placenta-associated genes moving in the opposite direction as the immune cellular genes. This set of genes was analyzed and determined to be highly correlated with outside temperature recorded at the weather station closest to the blood collection site at time of day of blood collection. FIG. 9B shows an example of high correlation between prediction of local outside temperature by gene modeling and actual temperatures recorded by weather station close to the blood collection site.

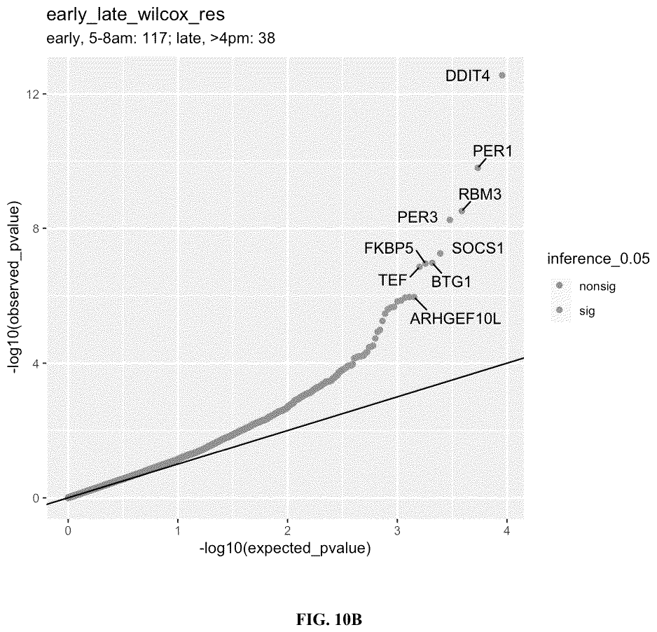

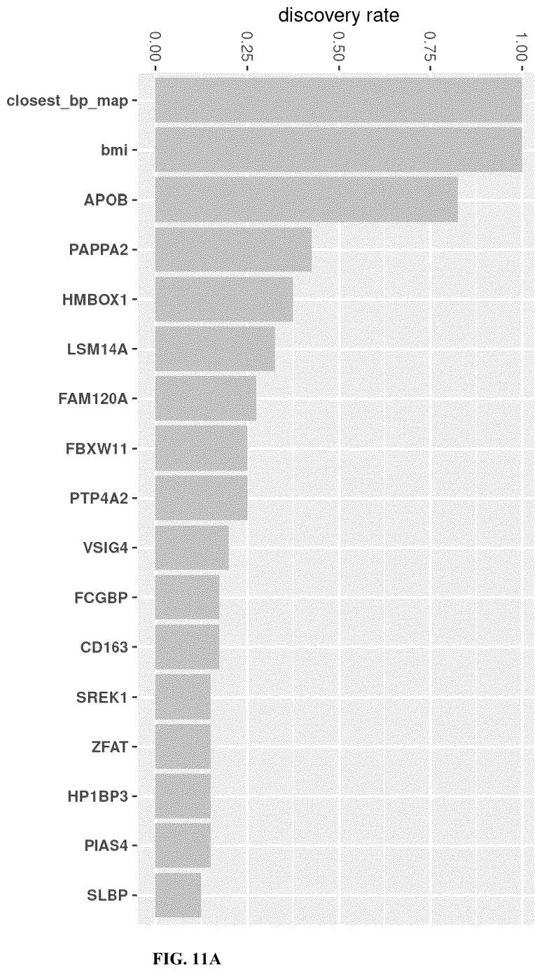

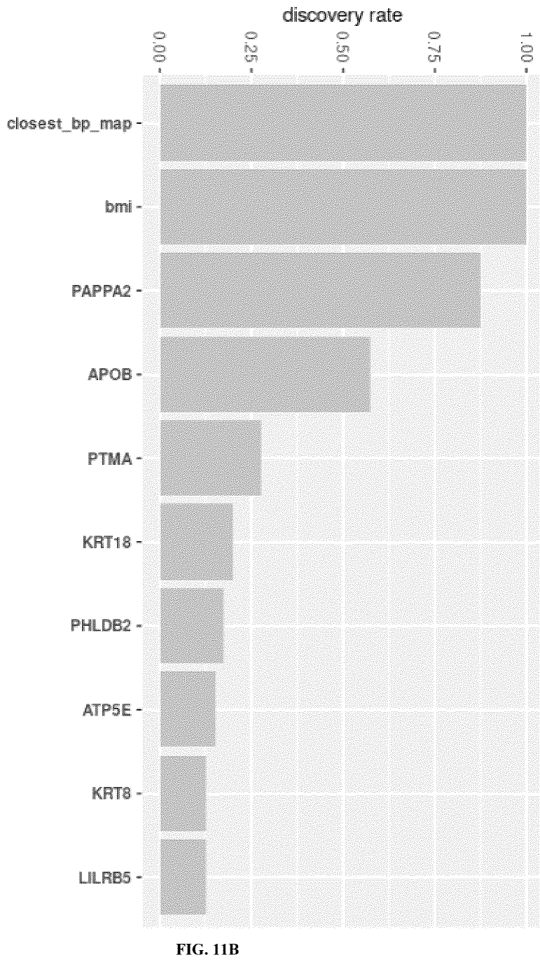

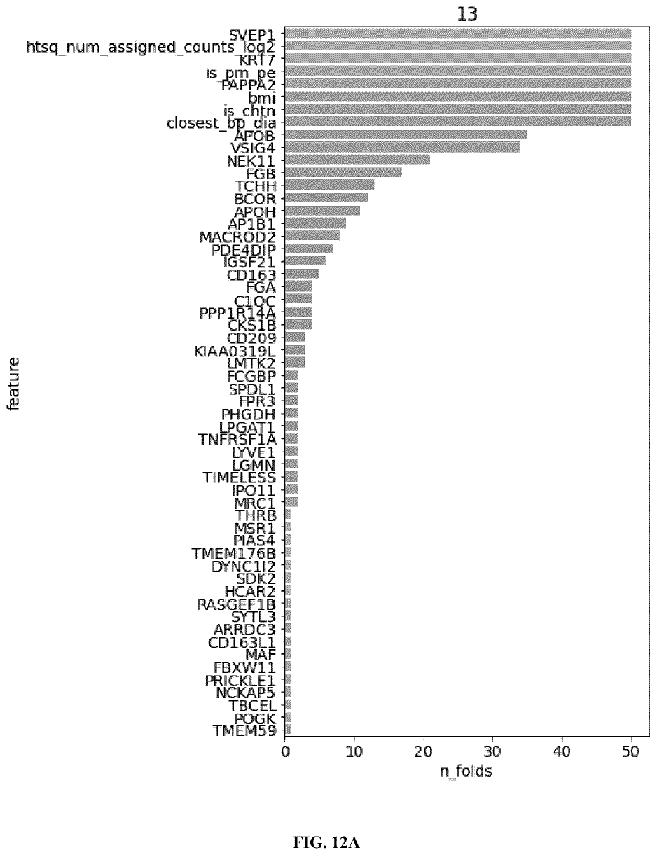

Additional analysis were performed to identify cfRNA variations in time of blood draw/collection associated with circadian clock, as shown in FIG. 10A. Samples were grouped by time of blood draw into morning (6 am to 10 am, n=121), midday (10 am to 2 pm, n=303), and afternoon (2 pm to 6 pm, n=113). Differential gene expression (DGE) analyses were performed to elucidate the impact of time of day. A quasi-likelihood negative binomial generalized log-linear model was fitted to count data using an edgeR package (v. 3.38.1), and DGE were discovered with an empirical Bayes quasi-likelihood F-test (edgeR) in all 3 possible pairwise comparisons. When looking for DGE between the morning and afternoon groups, 5,729 genes, or 43% of all analyzed genes, were determined to be significantly differentially expressed (FIG. 10B). For morning vs midday groups, 4,278 genes (32% of all analyzed genes) were determined to be DEG; and for midday vs afternoon groups, 15 genes (0.1% of all analyzed genes) were determined to be DGE. The three sets have very high overlap, ranging from 73% to 87% (p<10-30). Further, among the genes with highest separation reported were associated with circadian rhythm (e.g., PER1, PER3, DDIT4, FKBP5, RBM3, SOCS1, BTG1, and ARHGEF10L).

Further, biological variations associated with subject BMI, fetal fraction, and gestation age at blood collection are effectively regressed using similar techniques to increase power of gene discovery.

### Example 6: Early Prediction of Preeclampsia and Molecular Subtypes of Preeclampsia from a 3,036-Person Prospective Trial Using cfRNA Profiling

Using systems and methods of the present disclosure, early differentially expressed molecular markers of preeclampsia were identified in maternal blood samples using a prospective cohort of 3,036 cases described in Example 5. All samples were processed using a unified experimental and computational NGS pipeline for cell-free (cfRNA) sequencing. Twin pregnancies, cases of spontaneous preterm birth, and or preterm delivery based on non-preeclampsia diagnosis were excluded.

The study design was performed as follows. Two approaches were used. In the first, the cohort with preeclampsia cases were grouped by severity of preeclampsia diagnoses with medically-indicated deliveries to reduce the risk of composite adverse maternal outcomes for women. The severe preterm preeclampsia cases from cohort were grouped by delivery at less than 38 or 37 weeks of gestation age, as shown in FIGS. 8 and 9A-9B, respectively. In the second, observed major clinical factors based on USPSTF were added as candidate features in performing feature discovery for preterm preeclampsia cases, as shown in FIG. 7.

One approach to feature discovery is Sure Independence Screening, which ensures features that are orthogonal to each other to capture the biggest variation in the data. Using this method for the entire data set, a preterm preeclampsia signal was analyzed for delivering at less than 38 weeks gestational age. FIG. 11A and FIG. 11B show an example of the discovery rate for the genes associated with high risk developing preterm preeclampsia across a repeated cross-validation.

Table 5 provides a listing of differentially expressed genes discovered by Sure Independence Screening, as being predictive for the molecular subtype of preterm preeclampsia with medically-indicated delivery at less than 38 weeks. Similarly, Table 6 provides a listing of differentially expressed genes discovered by Sure Independence Screening, as being indicative for the molecular subtype of preterm preeclampsia with medically-indicated delivery for delivery at less than 37 weeks of gestation age.

TABLE 5
Preterm preeclampsia differentially expressed
genes discovered by Sure Independence Screening as
being predictive of delivery at less than 38 weeks

| | | | | | |
|---|---|---|---|---|---|
| PAPPA2 | APOB | FBXW11 | PTP4A2 | SLBP | UBE2Q1 |
| SREK1 | PISA4 | ZNF148 | PTMA | KRT18 | PHLDB2 |
| ATP5E | KRT8 | LILRB5 | HMBOX1 | LSM14A | FAM120A |
| PTP4A2 | VSIG4 | FCGBP | CD163 | ZFAT | HP1BP3 |
| RPLP1 | RPS27 | MAU2 | MACROD2 | SELENOP | GID4 |
| NSRP1 | NEK11 | SVEP1 | PAPPA | MAFF | CD163L1 |
| KISS1 | FABP1 | ANGPT2 | APOH | | |

TABLE 6
Preterm preeclampsia differentially expressed
genes discovered by Sure Independence Screening
as being predictive of delivery at less than 37 weeks

| | | | | | |
|---|---|---|---|---|---|
| PAPPA2 | KRT18 | SELENOP | TOMM5 | PAPPA | ATP5E |
| APOB | KRT8 | GID4 | VATIL | KISS1 | |

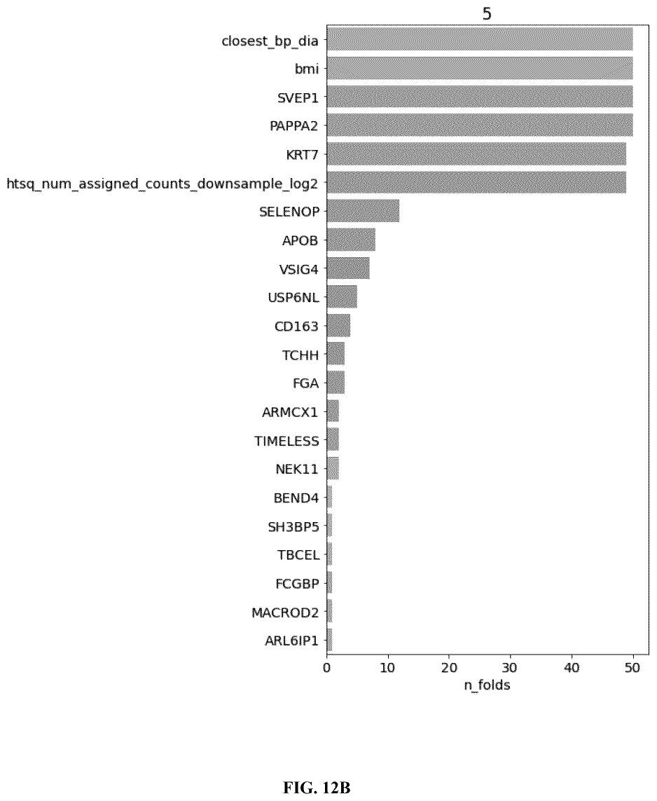

An alternative method for feature discovery is screening for differentially expressed genes in count spaces corrected for a variety of variables. Examples are correcting by BMI or total counts, or correcting by individual genes, such as KRT7 or SVEP1. Features are retained if they pass a multiple corrected p-value threshold and can be shown to add value to a model using the Aikake information criteria (AIC <−2). FIGS. 12A-12B depicts example outcomes for this type of modeling all gene markers and clinical factors discovered for preterm preeclampsia cases with delivery at less than 38 weeks or 37 weeks, respectively. Tables 7 and Table 8 provide a listing of all gene markers discovered by this approach for preterm preeclampsia cases with delivery at less than 38 weeks or 37 weeks, respectively.

TABLE 7
Preterm preeclampsia differentially expressed genes identified
by multiple space correction for deliveries at less than 38 weeks

| | | | | | |
|---|---|---|---|---|---|
| PAPPA2 | SVEP1 | KRT7 | VSIG4 | APOB | FGB |
| FCGBP | CD163 | TCHH | AXL | ETV5 | LYVE1 |
| IGSF21 | MACROD2 | FGG | CIQC | PPP1R14A | APOE |
| THRB | APOH | EFHD1 | TMEM176B | FABP1 | FPR3 |
| KISS1 | ALB | NEK11 | FGB | BCOR | AP1B1 |
| FGA | | | | | |

TABLE 8
Preterm preeclampsia genes identified by multiple
space correction for deliveries less than 37 weeks

| | | | | | |
|---|---|---|---|---|---|
| PAPPA2 | SELENOP | VSIG4 | APOB | CD163 | CD163L1 |
| TCHH | BCOR | FGA | USP6NL | SVEP1 | KRT7 |
| ARMCX1 | GATM | FPR3 | ETV5 | AXL | ZNF768 |
| TIMELESS | NEK11 | BEND4 | SH3BP5 | TBCEL | FCGBP |
| MACROD2 | ARL6IP1 | | | | |

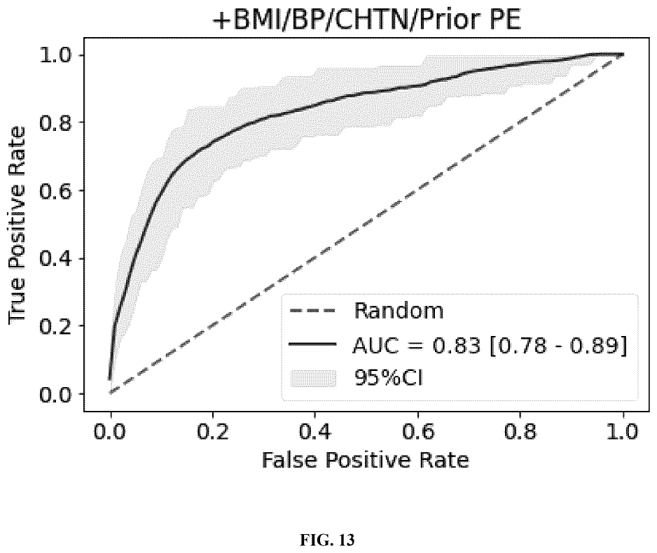

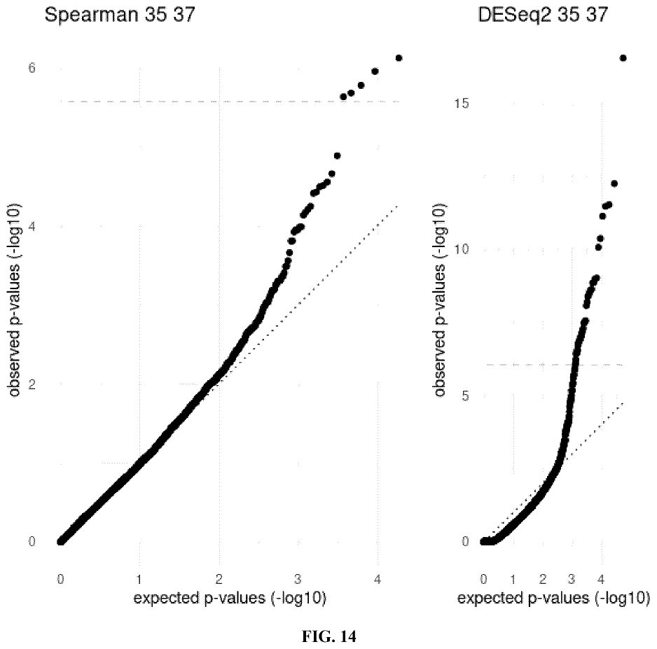

Modeling based on these features enabled the early prediction of preterm preeclampsia, yielding AUCs of up to 0.86 (for preterm preeclampsia cases with delivery at less than 36 weeks). FIG. 13 shows an example of an area-under-the-curve (AUC) for the ROC curve values with mean at 0.83 for ROC for a model predicting PE cases with deliveries at less than 37 weeks.

### Example 7: Early Prediction of Spontaneous Preterm Birth in a Prospective 3,036-Person Cohort Using Molecular Sub-Typing with cfRNA Profiling

Using systems and methods of the present disclosure, early differentially expressed molecular markers of preterm birth (PTB) were identified in maternal blood samples from a prospective cohort of 3,036 participants described in Example 5. All samples were processed using a unified experimental and computational NGS pipeline for cell-free (cfRNA) sequencing. Twin pregnancies, preterm birth cases of medically-indicated delivery based on preeclampsia diagnosis, and or other medically-indicated deliveries were excluded.

To identify differentially expressed gene markers for spontaneous preterm birth, two types of differential gene expression analyses were performed for two different molecular subtypes of spontaneous preterm birth, defined as either delivery at less than 35 weeks or delivery less than 37 weeks.

First, Spearman and DESeq2 differential gene expression analyses were performed for 55 spontaneous preterm birth cases with delivery before 35 weeks of gestational age and 2,899 full term birth control cases with delivery after 37 weeks of gestational age. FIG. 14 shows a quantile-quantile (QQ) plot for a differential gene expression signal for differentially expressed genes in pre-term birth cases with delivery before 35 weeks of gestational age. Table 9 shows a set of top 5 differentially expressed genes by Spearman ranked analyses for predicting spontaneous preterm birth cases with delivery earlier than 35 weeks of gestation.

TABLE 9
Set of top 5 differentially expressed genes that are
predictive for spontaneous preterm birth cases with delivery
earlier than 35 weeks using Spearman ranked analyses

| Index | Gene | P-value |
|---|---|---|
| 1 | ZNF812P | 0.01418108 |
| 2 | FPR3 | 0.020841643 |
| 3 | LILRB5 | 0.031239504 |
| 4 | CLEC9A | 0.039139074 |
| 5 | ETV5 | 0.043737094 |

Table 10 shows a set of top 43 differentially expressed genes by DESeq2 differential gene expression analyses for predicting spontaneous preterm birth cases with delivery earlier than 35 weeks of gestation.

TABLE 10
Set of top 43 differentially expressed genes that are
predictive for spontaneous preterm birth cases with delivery
earlier than 35 weeks using DESeq2 differential gene expression

| Index | Gene | P-value |
|---|---|---|
| 1 | LYVE1 | 1.66E−12 |
| 2 | TCHH | 3.24E−08 |
| 3 | CD163 | 1.68E−07 |
| 4 | DUSP27 | 1.91E−07 |
| 5 | VSIG4 | 4.21E−07 |
| 6 | MRC1 | 2.41E−06 |
| 7 | GPR82 | 4.81E−06 |
| 8 | GPR34 | 5.36E−05 |
| 9 | MSR1 | 5.74E−05 |
| 10 | RNASE1 | 7.74E−05 |
| 11 | TREM2 | 7.86E−05 |
| 12 | FPR3 | 1.31E−04 |
| 13 | FCGBP | 1.44E−04 |
| 14 | MERTK | 1.71E−04 |
| 15 | STAB1 | 2.13E−04 |
| 16 | LILRB5 | 2.29E−04 |
| 17 | CADM1 | 3.60E−04 |
| 18 | SELENOP | 4.75E−04 |
| 19 | NCKAP5 | 0.00155276 |
| 20 | C1QC | 0.00160502 |
| 21 | C1QB | 0.00182701 |
| 22 | DBNDD2 | 0.00187367 |
| 23 | MMP2 | 0.00287475 |
| 24 | AXL | 0.00317878 |
| 25 | GATM | 0.00387293 |
| 26 | FOLR2 | 0.00412276 |
| 27 | ZNF812P | 0.00530928 |
| 28 | ETV5 | 0.00546725 |
| 29 | MTCO1P12 | 0.00637143 |
| 30 | CLEC9A | 0.00696217 |
| 31 | MMP14 | 0.00759331 |
| 32 | CD209 | 0.0080383 |
| 33 | ZFHX3 | 0.00868013 |
| 34 | EPS8 | 0.00933179 |
| 35 | SLCO2B1 | 0.01086637 |
| 36 | OLFML3 | 0.01562314 |
| 37 | CD276 | 0.0170661 |
| 38 | CABLES1 | 0.01958957 |
| 39 | SDC3 | 0.01977068 |
| 40 | MAF | 0.02108906 |
| 41 | RGL1 | 0.03271522 |
| 42 | SPRED1 | 0.04326907 |
| 43 | UNC13C | 0.04952278 |

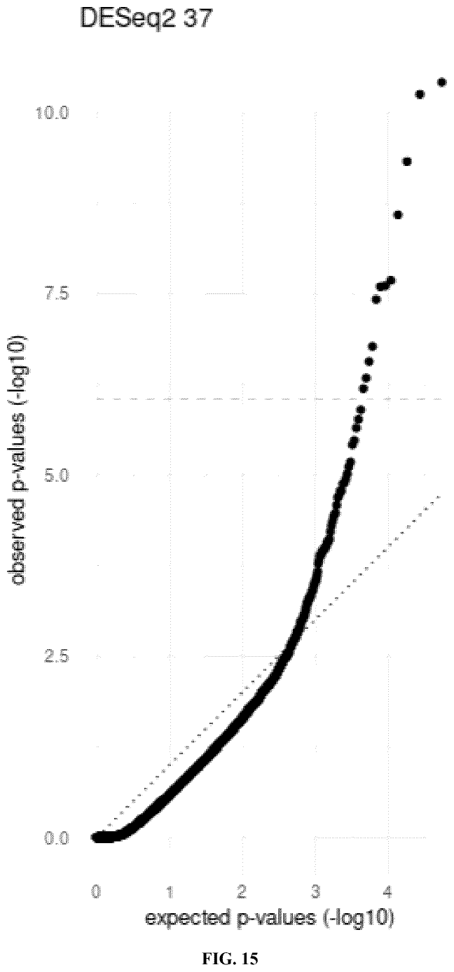

Similar analyses with DESeq2 differential gene expression were performed for 135 spontaneous preterm birth cases with spontaneous birth before 37 weeks of gestational age and 2,899 full term birth control cases with delivery after 37 weeks of gestational age. FIG. 15 shows a quantile-quantile (QQ) plot for a differential gene expression signal for differentially expressed genes in pre-term birth cases with delivery before 37 weeks of gestational age.

Table 11 shows a set of top 12 differentially expressed genes by DESeq2 differential gene expression analyses for predicting spontaneous preterm birth cases with delivery earlier than 37 weeks of gestation.

TABLE 11
Set of 12 top differentially expressed genes that are predictive
for spontaneous preterm birth cases with delivery
earlier than 37 weeks using DESeq2 differential gene expression

| Index | Gene | P-value |
|---|---|---|
| 1 | TCHH | 2.15E−06 |
| 2 | LYVE1 | 3.15E−06 |
| 3 | GPR82 | 2.65E−05 |
| 4 | CD163 | 1.45E−04 |
| 5 | SAA2 | 0.001162 |
| 6 | DUSP27 | 0.001376 |
| 7 | CSRP3 | 0.00141 |
| 8 | VSIG4 | 0.002136 |
| 9 | GPR34 | 0.009453 |
| 10 | MRC1 | 0.015219 |
| 11 | FPR3 | 0.025791 |
| 12 | ADGRG7 | 0.036084 |

### Example 8: Prospective Cohort Subjects for Preeclampsia, Fetal Growth Restriction, Gestational Diabetes Case Studies and cfRNA NGS Data Correction with Sequencing Quality Filters

The cohort of 3,036 samples described in Example 5 was further filtered for samples with high quality sequencing metrics assigned counts for additional discovery and analysis.

2970 samples had complete medical records and passed 5.5M assigned counts quality metrics, and were used for discovery and modeling for preeclampsia. Table 12 shows the demographic and clinical data metrics for the preeclampsia studies, including 2634 healthy participants and 336 participants diagnosed with preeclampsia, which were collected based on U.S. Preventive Services Task Force (USPSTF) guidelines and recommendations. Various preeclampsia risk factors were recorded for this cohort, including: parity; race; chronic hypertension (chtn); diabetic status excluding gestational diabetes (diabetic_not_gdm); mothers age; body mass index (bmi); prior preeclampsia diagnosis (pm_pe); U.S. Preventive Services Taskforce (USPSTF) risk level (www.uspreventiveservicestaskforce.org/uspstf/recommendation/preeclampsia-screening); and artificial in vitro fertilization (IVF).

TABLE 12
Demographic and clinical data metrics for the preeclampsia studies.

| | FALSE | TRUE | |
|---|---|---|---|
| | (N = 2634) | (N = 336) | P-value |
| is_pm_pe | | | |
| Yes | 180 (6.8%) | 72 (21.4%) | <0.001 |
| No | 2454 (93.2%) | 264 (78.6%) | |
| is_gdm | | | |
| Yes | 248 (9.4%) | 61 (18.2%) | <0.001 |
| No | 2385 (90.5%) | 275 (81.8%) | |
| Missing | 1 (0.0%) | 0 (0%) | |
| is_chtn | | | |
| Yes | 153 (5.8%) | 64 (19.0%) | <0.001 |
| No | 2481 (94.2%) | 272 (81.0%) | |
| parity | | | |
| multi | 1638 (62.2%) | 158 (47.0%) | <0.001 |
| null | 996 (37.8%) | 178 (53.0%) | |
| major_race | | | |
| asian | 108 (4.1%) | 9 (2.7%) | 0.0438 |
| black | 625 (23.7%) | 102 (30.4%) | |
| hispanic | 381 (14.5%) | 53 (15.8%) | |
| multiracial | 240 (9.1%) | 24 (7.1%) | |
| white | 1280 (48.6%) | 148 (44.0%) | |
| collection_ga | | | |
| Mean (SD) | 19.9 (1.11) | 19.8 (1.12) | 0.101 |
| Median [Min, Max] | 20.0 [17.6, 22.0] | 19.9 [17.6, 21.9] | |
| delivery_ga | | | |
| Mean (SD) | 38.7 (2.09) | 37.1 (2.52) | <0.001 |
| Median [Min, Max] | 39.1 [19.1, 42.3] | 37.4 [24.0, 41.7] | |
| age | | | |
| Mean (SD) | 30.3 (5.62) | 30.2 (6.33) | 0.889 |
| Median [Min, Max] | 30.5 [18.0, 45.5] | 30.4 [18.1, 44.0] | |
| bmi | | | |
| Mean (SD) | 28.9 (7.30) | 32.3 (8.68) | <0.001 |
| Median [Min, Max] | 27.4 [12.8, 63.5] | 31.0 [18.3, 65.4] | |
| Missing | 4 (0.2%) | 0 (0%) | |
| tmax_c | | | |
| Mean (SD) | 23.5 (8.25) | 24.3 (7.30) | 0.0638 |
| Median [Min, Max] | 24.9 [−7.20, 46.7] | 25.2 [−1.00, 39.0] | |
| Missing | 24 (0.9%) | 1 (0.3%) | |

2,803 samples had complete medical records and passed 5 million assigned counts quality metrics and used for discovery and modeling for fetal growth restriction observational studies. Table 13 shows the distribution of demographic and clinical data metrics for the fetal growth restriction studies, including 2745 healthy participants and 58 participants with fetal growth restriction cases to fall within the 3rd baby weight percentile (SGA3) and annotated in the following as SGA3. Data were collected based guideline and recommendation by Society for Maternal-Fetal Medicine (SMFM (www.smfm.org/publications/289-smfm-consult-series-52-diagnosis-and-management-of-fetal-growth-restriction).

TABLE 13
Demographic and clinical data metrics for the
fetal growth restriction studies.

| | FALSE | TRUE | |
|---|---|---|---|
| | (N = 2745 | (N = 58) | P-value |
| is_pe | | | |
| Yes | 314 (11.4%) | 11 (19.0%) | 0.118 |
| No | 2431 (88.6%) | 47 (81.0%) | |
| is_pm_pe | | | |
| Yes | 234 (8.5%) | 2 (3.4%) | 0.255 |
| No | 2511 (91.5%) | 56 (96.6%) | |
| is_gdm | | | |
| Yes | 301 (11.0%) | 1 (1.7%) | 0.0421 |
| No | 2443 (89.0%) | 57 (98.3%) | |
| Missing | 1 (0.0%) | 0 (0%) | |
| is_chtn | | | |
| Yes | 207 (7.5%) | 6 (10.3%) | 0.584 |
| No | 2538 (92.5%) | 52 (89.7%) | |
| parity | | | |
| multi | 1658 (60.4%) | 23 (39.7%) | 0.00225 |
| null | 1087 (39.6%) | 35 (60.3%) | |
| major_race | | | |
| asian | 111 (4.0%) | 0 (0%) | <0.001 |
| black | 656 (23.9%) | 34 (58.6%) | |
| hispanic | 406 (14.8%) | 1 (1.7%) | |
| multiracial | 251 (9.1%) | 5 (8.6%) | |
| white | 1321 (48.1%) | 18 (31.0%) | |
| collection_ga | | | |
| Mean (SD) | 19.9 (1.12) | 19.9 (1.20) | 0.731 |
| Median [Min, Max] | 20.0 [17.6, 22.0] | 20.1 [17.7, 21.9] | |
| delivery_ga | | | |
| Mean (SD) | 38.6 (1.92) | 37.2 (3.96) | 0.00892 |
| Median [Min, Max] | 39.0 [22.0, 41.9] | 38.2 [23.0, 41.1] | |
| age | | | |
| Mean (SD) | 30.3 (5.66) | 28.6 (6.38) | 0.0418 |
| Median [Min, Max] | 30.6 [18.0, 45.5] | 28.6 [18.1, 41.7] | |
| bmi | | | |
| Mean (SD) | 29.3 (7.54) | 28.7 (8.69) | 0.624 |
| Median [Min, Max] | 27.8 [12.8, 65.4] | 26.5 [15.2, 61.5] | |
| Missing | 10 (0.4%) | 1 (1.7%) | |
| Mean (SD) | 23.9 (8.01) | 20.1 (11.0) | 0.00985 |
| Median [Min, Max] | 25.2 [−7.20, 46.7] | 22.7 [−5.60, 36.4] | |
| Missing | 23 (0.8%) | 0 (0%) | |

2,079 samples had complete medical records and passed 5 million assigned counts quality metrics and used for discovery and modeling for of gestational diabetes mellitus (GDM observational studies.

Table 14 shows the distribution of demographic and clinical factors within this cohort including 2004 healthy participants and 175 participants with gestational diabetes mellitus (GDM). Data were collected based on U.S. Preventive Services Task Force (USPSTF) guidelines and recommendations. (www.uspreventiveservicestaskforce.org/uspstf/recommendation/gestational-diabetes-screening).

TABLE 14
Demographic and clinical data metrics for the
gestational diabetes mellitus (GDM) studies.

| | FALSE | TRUE | |
|---|---|---|---|
| | (N = 2004) | (N = 175) | P-value |
| is_pe | | | |
| Yes | 207 (10.3%) | 33 (18.9%) | <0.001 |
| No | 1797 (89.7%) | 141 (80.6%) | |
| Missing | 0 (0%) | 1 (0.6%) | |
| is_chtn | | | |
| Yes | 114 (5.7%) | 20 (11.4%) | 0.00414 |
| No | 1890 (94.3%) | 155 (88.6%) | |
| is_pm_pe | | | |
| Yes | 147 (7.3%) | 17 (9.7%) | 0.32 |
| No | 1857 (92.7%) | 158 (90.3%) | |
| parity | | | |
| multi | 1169 (58.3%) | 111 (63.4%) | 0.218 |
| null | 835 (41.7%) | 64 (36.6%) | |
| major_race | | | |
| asian | 82 (4.1%) | 6 (3.4%) | 0.338 |
| black | 477 (23.8%) | 36 (20.6%) | |
| hispanic | 273 (13.6%) | 20 (11.4%) | |
| multiracial | 205 (10.2%) | 14 (8.0%) | |
| white | 967 (48.3%) | 99 (56.6%) | |
| collection_ga | | | |
| Mean (SD) | 19.8 (1.11) | 19.9 (1.13) | 0.608 |
| Median [Min, Max] | 20.0 [17.6, 22.0] | 20.0 [17.6, 21.9] | |
| delivery_ga | | | |
| Mean (SD) | 38.7 (2.26) | 38.0 (1.88) | <0.001 |
| Median [Min, Max] | 39.1 [19.1, 42.3] | 38.6 [25.4, 40.7] | |
| Missing | 4 (0.2%) | 0 (0%) | |
| age | | | |
| Mean (SD) | 29.9 (5.65) | 31.8 (5.39) | <0.001 |
| Median [Min, Max] | 30.2 [18.0, 45.1] | 32.0 [19.6, 43.6] | |
| bmi | | | |
| Mean (SD) | 28.4 (7.22) | 32.2 (8.05) | <0.001 |
| Median [Min, Max] | 26.9 [12.8, 65.4] | 31.4 [15.5, 59.9] | |
| Missing | 8 (0.4%) | 2 (1.1%) | |
| tmax_c | | | |
| Mean (SD) | 24.0 (8.16) | 25.7 (7.19) | 0.0036 |
| Median [Min, Max] | 25.4 [−7.20, 46.7] | 27.4 [−6.70, 36.1] | |
| Missing | 17 (0.8%) | 3 (1.7%) | |

### Example 9: Early Prediction of Gestational Diabetes Mellitus (GDM) Using Molecular Subtyping from a 2179 Person Prospective Trial Using cfRNA Profiling

Using systems and methods of the present disclosure, early differentially expressed molecular markers of gestational diabetes mellitus (GDM) were identified in maternal blood samples using a prospective cohort of 2179 described in Example 8. All samples were processed using a unified experimental and computational NGS pipeline for cell-free (cfRNA) sequencing. Twin pregnancies, samples collected outside a collection window of 17.5-22 weeks gestational age, samples with less than 5 million assigned counts, and samples showing signs of contamination or significant hemolysis were marked as ineligible and removed.

Additionally, 67 cases of diagnosed pre-existing type I/type II diabetes, 29 cases of severe first-trimester glucose tolerance test (which may indicate undiagnosed pre-existing diabetes, 37 cases of significantly elevated hemoglobin A1c (HbA1c), 120 individuals who failed a glucose tolerance test but are not marked as having gestational diabetes, and 70 individuals marked as having gestational diabetes without a glucose tolerance test failure were also excluded, bringing the total valid samples to 2179. Table 15 shows the breakdown of GDM diagnosed cases based on oral glucose tolerance test (OGTT) with an abnormal reading.

TABLE 15
GDM diagnosed cases based on oral glucose
tolerance test (OGTT) with an abnormal reading.

| OGTT test type | Number of GDM cases |
|---|---|
| 3-Hr OGTT, at least 1 abnormal reading | 160 |
| 2-Hr OGTT, at least 1 abnormal reading | 34 |
| 1-Hr OGTT over 200 | 20 |

Profiling of cfRNA can be implemented using various statistical and machine-learning techniques, including differential gene expression analysis, supervised feature selection, unsupervised feature selection, feature engineering, latent-space analysis, manifold learning, and deep learning, among others. In this example, cfRNA profiling was performed using differential gene expression analysis. In this example, the DESeq2 R package was used to conduct the differential gene expression analysis, which fit a generalized linear model for each gene, estimating gene-wise differences in cfRNA between GDM cases and controls.

When profiling cfRNA, covariates may be used to improve the discovery of relevant genes. In this example, we used several covariates to better resolve genes differentially expressed in GDM. A correction was included for the model of the sequencing machine (e.g. “nextseq”, “novaseq”) and the wave (giga-batch) of sequencing, as well as for an estimation of total process efficiency using ERCC (External RNA Controls Consortium) RNA spike-in controls that were added into the samples. Additionally, we corrected for estimated fetal fraction using counts of the VGLL3 gene, which is highly correlated with Y-chromosome genes in male babies (where Y-chromosome reads are assumed to come from the fetus rather than the maternal subject), but is not itself on the Y-chromosome.

Though not used in this example, other covariates may be used to improve the discovery of relevant genes, such as total assigned counts, total reads, sequence duplication rate, mitochondrial gene proportion, intronic gene proportion, intragenic proportion, the temperature of the shipping box containing the sample (this is recorded by a sensor during transit), maternal hemoglobin content, the sequencing batch, the hybridization/capture batch, the library preparation batch, the extraction batch, the state where the sample was collected, the hospital where the sample was collected, or the identity of the lab operators/technicians implementing the sample preparation. These covariates may be non-obvious. In some respects, these covariates may be used to improve predictive modeling of GDM and other indications.

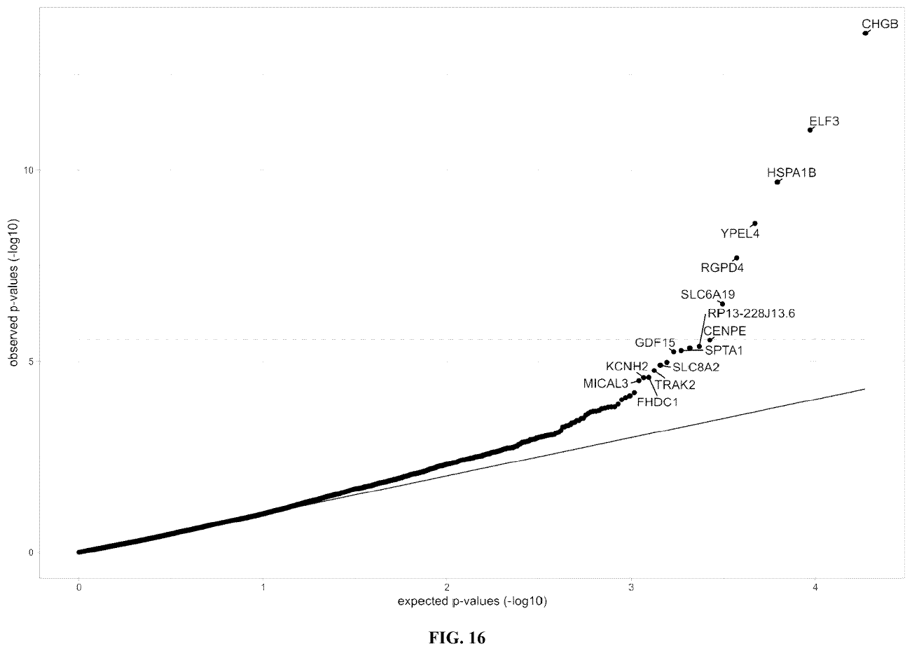

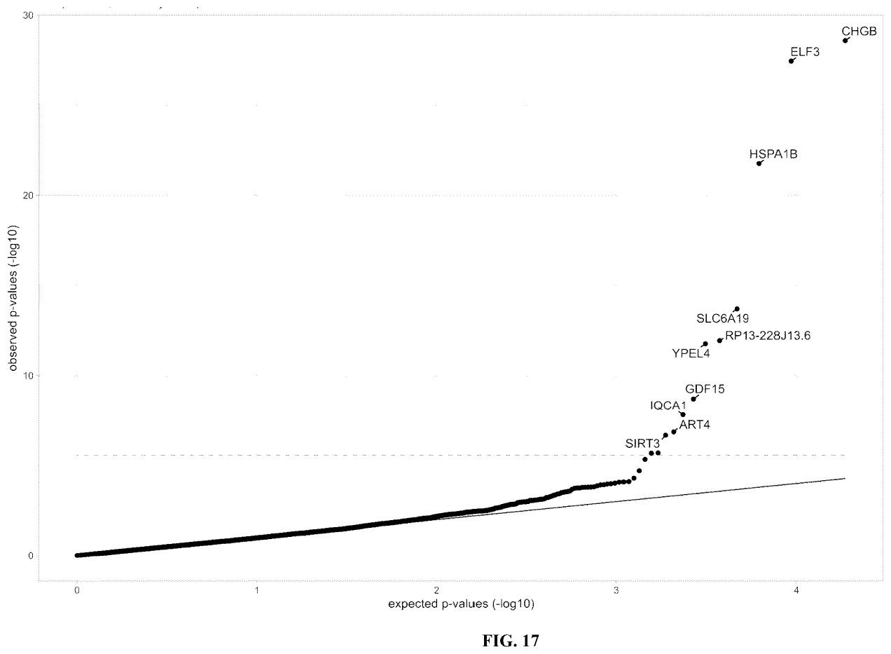

After differential analysis, 26 genes shown in Table 16 were identified as correlating to the risk of developing gestational diabetes, after correcting p-values for multiple comparisons using Holm's method. FIG. 16 shows a quantile-quantile (QQ) plot for a differential expression signal for differentially expressed genes in GDM cases. After additionally correcting for BMI, an additional PRC1 gene has been identified for predicting risk of developing GDM after correcting p-values for multiple comparisons using Holm's method.

TABLE 16
A set of 26 top differentially expressed genes that were
identified to correlate to the risk of developing
gestational diabetes after correcting p-values for
multiple comparisons using Holm's method.

| Index | Gene | P-value | P adj. |
|---|---|---|---|
| 1 | CHGB | 9.22E−21 | 1.72E−16 |
| 2 | ELF3 | 5.13E−17 | 9.58E−13 |
| 3 | HSPA1B | 5.07E−15 | 9.46E−11 |
| 4 | YPEL4 | 2.05E−13 | 3.82E−09 |
| 5 | RGPD4 | 4.36E−12 | 8.14E−08 |
| 6 | SLC6A19 | 2.57E−10 | 4.79E−06 |
| 7 | CENPE | 6.46E−09 | 1.21E−04 |
| 8 | RP13-228J13.6 | 1.15E−08 | 2.14E−04 |
| 9 | ANKLE1 | 1.32E−08 | 2.47E−04 |
| 10 | SPTA1 | 1.64E−08 | 3.06E−04 |
| 11 | GDF15 | 1.84E−08 | 3.43E−04 |
| 12 | ART4 | 4.62E−08 | 8.62E−04 |
| 13 | SLC8A2 | 5.94E−08 | 1.11E−03 |
| 14 | TRAK2 | 9.63E−08 | 1.80E−03 |
| 15 | FHDC1 | 1.80E−07 | 3.35E−03 |
| 16 | KCNH2 | 1.83E−07 | 3.42E−03 |
| 17 | MICAL3 | 2.39E−07 | 4.46E−03 |
| 18 | TUBG1 | 6.78E−07 | 1.26E−02 |
| 19 | LIFR | 9.10E−07 | 1.70E−02 |
| 20 | TCHH | 1.07E−06 | 2.00E−02 |
| 21 | HMBS | 1.30E−06 | 2.42E−02 |
| 22 | CD163 | 1.93E−06 | 3.60E−02 |
| 23 | ETFDH | 2.45E−06 | 4.58E−02 |
| 24 | CENPF | 2.48E−06 | 4.63E−02 |
| 25 | TIMELESS | 2.54E−06 | 4.73E−02 |
| 26 | PRC1 | 2.34E−06 | 4.38E−02 |

Firstline treatment for GDM may include lifestyle interventions, including nutritional counseling, dietary changes, and daily exercise, with the goal of decreasing postprandial hyperglycemia. Once the patient commences an appropriate diet and exercise plan, close surveillance of blood glucose levels may be necessary to ensure glycemic control is maintained. This may be accomplished with patients performing daily self-monitored blood glucose checks. Once pregnant subjects achieve and maintain good glycemic control, the frequency of testing can be decreased. If these targets cannot be met and the majority of fasting and/or postprandial values are elevated, then pharmacotherapy may be recommended.

A challenge may be that at least 1 in 4 women with GDM do not respond to lifestyle intervention, including diet and exercise, and require pharmacotherapy to achieve euglycemia. If healthy eating and being active are not enough to manage a patient's blood sugar, physicians may prescribe medication (e.g. insulin and/or metformin) to manage hypoglycemia. Both the ACOG and ADA recommend insulin as first-line therapy because it does not cross the placenta and improves perinatal outcomes. The Society for Maternal-Fetal Medicine recommends either metformin or insulin as reasonable first-line options.

To perform molecular subtype analysis, the cases of GDM were divided into two distinctive classification groups. Cases of type A1 GDM (GDMAT) may be managed with diet and exercise, and cases of A2 type GDM (GDMA2) may require pharmacotherapy to manage hypoglycemia. By modeling each subtype separately, we may improve predictive modeling for that subtype, or may discover relevant genes that were obscured by the initial participant heterogeneity. In some respects, a subtype-sensitive analysis strategy may improve predictive modeling of GDM. In the following example analysis of each GDM subtype, we used the same procedures and covariates as in the general GDM analysis described above.

After differential analysis, 13 genes shown in Table 17 were identified to relate to the risk of developing GDMA1 type. FIG. 17 shows quantile-quantile (QQ) plot for a differential expression signal for differentially expressed genes in GDMA1 cases. Five of these genes (IQCA1, SIRT3, IGHV3-49, PIGR, and RPL10P9) were not identified in the primary GDM analysis, indicating possible uniqueness for molecular subtype of GDMA1. Many of the most significant genes in the primary GDM analysis (CHGB, ELF3, HSPA1B, and SLC6A19) showed a higher log-fold change in this GDM manageable with diet and exercise. In some respects, these novel genes and subtype-enriched genes may be used to improve predictive modeling for GDM.

TABLE 17
A set of 13 top differentially expressed genes that were
identified to correlate to the risk of developing gestational
diabetes type A1 (GDMA1) after correcting
p-values for multiple comparisons using Holm's method.

| Index | Gene | p-value | p adj. |
|---|---|---|---|
| 1 | CHGB | 3.45E−32 | 6.45E−28 |
| 2 | ELF3 | 6.09E−31 | 1.14E−26 |
| 3 | HSPA1B | 1.13E−24 | 2.11E−20 |
| 4 | SLC6A19 | 8.65E−16 | 1.61E−11 |
| 5 | RP13-228J13.6 | 7.45E−14 | 1.39E−09 |
| 6 | YPEL4 | 1.15E−13 | 2.15E−09 |
| 7 | GDF15 | 2.69E−10 | 5.02E−06 |
| 8 | IQCA1 | 2.54E−09 | 4.74E−05 |
| 9 | ART4 | 2.87E−08 | 5.35E−04 |
| 10 | SIRT3 | 4.57E−08 | 8.53E−04 |
| 11 | IGHV3-49 | 5.46E−07 | 1.02E−02 |
| 12 | PIGR | 5.68E−07 | 1.06E−02 |
| 13 | RPL10P9 | 1.36E−06 | 2.54E−02 |

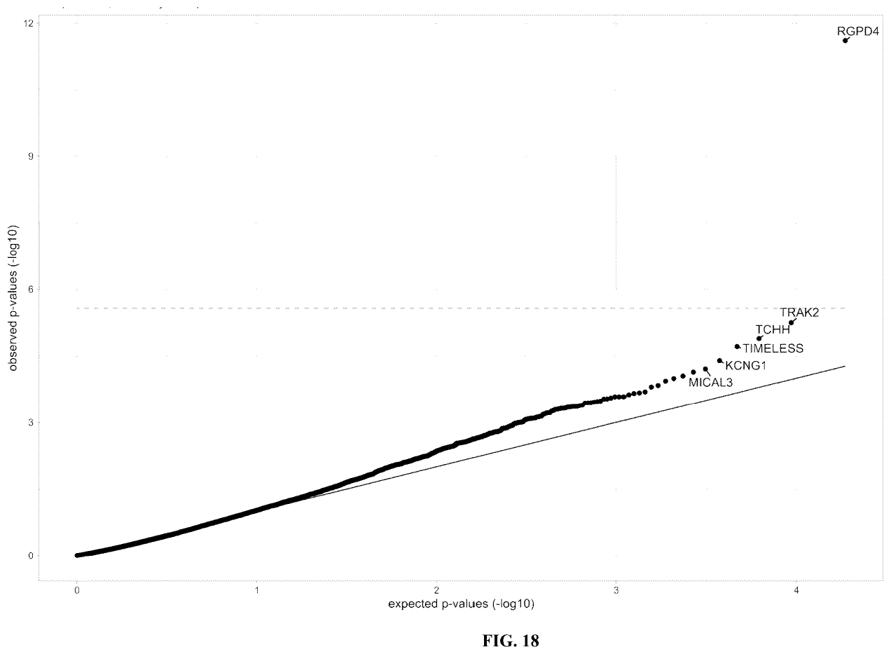

After differential analysis, 70 genes shown in Table 18 were identified to correlate with the risk of developing GDM type A2 (GDMA2) requiring pharmacotherapy. The top 20 most significant genes are: RGPD4, TRAK2, TCHH, TIMELESS, KCNG1, MICAL3, ABCG1, INCENP, FHDC1, CD163, CDKN2C, NET1, STAB1, CENPF, KIF15, SPAG5, CDC20, TPX2, PLK1, and CTD-3099C6.9. FIG. 18 shows a quantile-quantile (QQ) plot for a differential expression signal for differentially expressed genes in GDMA2 type.

The majority of these genes were not identified in the primary GDM analysis or the GDMA1 type analysis, indicating possible uniqueness for the molecular subtype of cases of GDMA2 type. In some respects, these subtype-specific genes may be used to improve predictive modeling for GDM.

TABLE 18
A set of 70 top differentially expressed genes that were
identified to correlate to the risk of developing gestational
diabetes type A2 (GDMA2) after correcting
p-values for multiple comparisons using Holm's method.

| Index | Gene | p-value | p adj. |
|---|---|---|---|
| 1 | RGPD4 | 1.17E−22 | 2.18E−18 |
| 2 | TRAK2 | 1.23E−10 | 2.29E−06 |
| 3 | TCHH | 5.74E−10 | 1.07E−05 |
| 4 | TIMELESS | 1.25E−09 | 2.34E−05 |
| 5 | KCNG1 | 4.97E−09 | 9.27E−05 |
| 6 | MICAL3 | 1.12E−08 | 2.09E−04 |
| 7 | ABCG1 | 1.53E−08 | 2.85E−04 |
| 8 | INCENP | 2.25E−08 | 4.20E−04 |
| 9 | FHDC1 | 2.88E−08 | 5.37E−04 |
| 10 | CD163 | 3.70E−08 | 6.91E−04 |
| 11 | CDKN2C | 5.69E−08 | 1.06E−03 |
| 12 | NET1 | 6.61E−08 | 1.23E−03 |
| 13 | STAB1 | 1.07E−07 | 2.00E−03 |
| 14 | CENPF | 1.18E−07 | 2.20E−03 |
| 15 | KIF 15 | 1.25E−07 | 2.34E−03 |
| 16 | SPAG5 | 1.42E−07 | 2.65E−03 |
| 17 | CDC20 | 1.71E−07 | 3.19E−03 |
| 18 | TPX2 | 1.73E−07 | 3.23E−03 |
| 19 | PLK1 | 1.73E−07 | 3.24E−03 |
| 20 | CTD-3099C6.9 | 1.96E−07 | 3.65E−03 |
| 21 | SMARCA4 | 2.13E−07 | 3.97E−03 |
| 22 | HIST1H2BM | 2.15E−07 | 4.02E−03 |
| 23 | PEG10 | 2.64E−07 | 4.93E−03 |
| 24 | AFF3 | 2.80E−07 | 5.21E−03 |
| 25 | CRISP3 | 2.93E−07 | 5.47E−03 |
| 26 | MCM2 | 3.12E−07 | 5.82E−03 |
| 27 | NCAPD2 | 3.16E−07 | 5.89E−03 |
| 28 | SPTA1 | 3.19E−07 | 5.94E−03 |
| 29 | MYO18A | 3.95E−07 | 7.37E−03 |
| 30 | IQGAP3 | 4.13E−07 | 7.69E−03 |
| 31 | FAM129C | 4.42E−07 | 8.23E−03 |
| 32 | PRC1 | 4.46E−07 | 8.31E−03 |
| 33 | BZW1 | 4.51E−07 | 8.41E−03 |
| 34 | KIF18B | 4.70E−07 | 8.75E−03 |
| 35 | WDFY4 | 4.85E−07 | 9.03E−03 |
| 36 | NEK2 | 5.19E−07 | 9.67E−03 |
| 37 | LARGE1 | 5.33E−07 | 9.92E−03 |
| 38 | BCL11A | 5.34E−07 | 9.95E−03 |
| 39 | CUL9 | 5.70E−07 | 1.06E−02 |
| 40 | MCM5 | 5.99E−07 | 1.12E−02 |
| 41 | ACADVL | 6.09E−07 | 1.13E−02 |
| 42 | ANKLE1 | 6.69E−07 | 1.25E−02 |
| 43 | KCNH2 | 7.14E−07 | 1.33E−02 |
| 44 | ADD2 | 8.32E−07 | 1.55E−02 |
| 45 | SIPA1L3 | 8.43E−07 | 1.57E−02 |
| 46 | PM20D1 | 8.70E−07 | 1.62E−02 |
| 47 | COL19A1 | 9.30E−07 | 1.73E−02 |
| 48 | ACOT11 | 9.95E−07 | 1.85E−02 |
| 49 | KPNA2 | 1.17E−06 | 2.18E−02 |
| 50 | CDCA7L | 1.20E−06 | 2.23E−02 |
| 51 | AXL | 1.27E−06 | 2.36E−02 |
| 52 | IL12RB2 | 1.29E−06 | 2.41E−02 |
| 53 | ARHGAP11A | 1.39E−06 | 2.60E−02 |
| 54 | SELENOP | 1.41E−06 | 2.62E−02 |
| 55 | BACH2 | 1.43E−06 | 2.66E−02 |
| 56 | PFKL | 1.44E−06 | 2.68E−02 |
| 57 | PAX5 | 1.50E−06 | 2.80E−02 |
| 58 | FCGBP | 1.51E−06 | 2.81E−02 |
| 59 | FOXM1 | 1.60E−06 | 2.98E−02 |
| 60 | PLCB1 | 1.61E−06 | 3.00E−02 |
| 61 | SMC2 | 1.76E−06 | 3.27E−02 |
| 62 | CIT | 2.03E−06 | 3.77E−02 |
| 63 | ETFDH | 2.09E−06 | 3.90E−02 |
| 64 | WHSC1 | 2.12E−06 | 3.94E−02 |
| 65 | CCNF | 2.17E−06 | 4.04E−02 |
| 66 | NUMA1 | 2.19E−06 | 4.07E−02 |
| 67 | OLFM4 | 2.31E−06 | 4.30E−02 |
| 68 | SHCBP1 | 2.38E−06 | 4.43E−02 |
| 69 | CKAP2L | 2.40E−06 | 4.47E−02 |
| 70 | KIF20A | 2.57E−06 | 4.78E−02 |

### Example 10: Early Prediction of Fetal Growth Restriction Using Molecular Sub-Typing from a 2803 Person Prospective Trial Using cfRNA Profiling

Using systems and methods of the present disclosure, early differentially expressed molecular markers of fetal growth restriction were identified in maternal blood samples using a prospective cohort of 3,036 cases described in Example 8. All samples were processed using a unified experimental and computational NGS pipeline for cell-free (cfRNA) sequencing. Twin pregnancies, cases of spontaneous preterm birth, and or preterm delivery based on non-preeclampsia diagnosis were excluded. Restricting sample collection within a 17.5-22 weeks gestational age window, and further excluding samples with less than 5 million assigned counts and signs of contamination through hemolysis, reduces the sample size to 2803 participants. To threshold the signal to noise ratio of gene counts, we imposed a threshold of median gene counts of at least 5, resulting in a feature space of 13,734 genes.

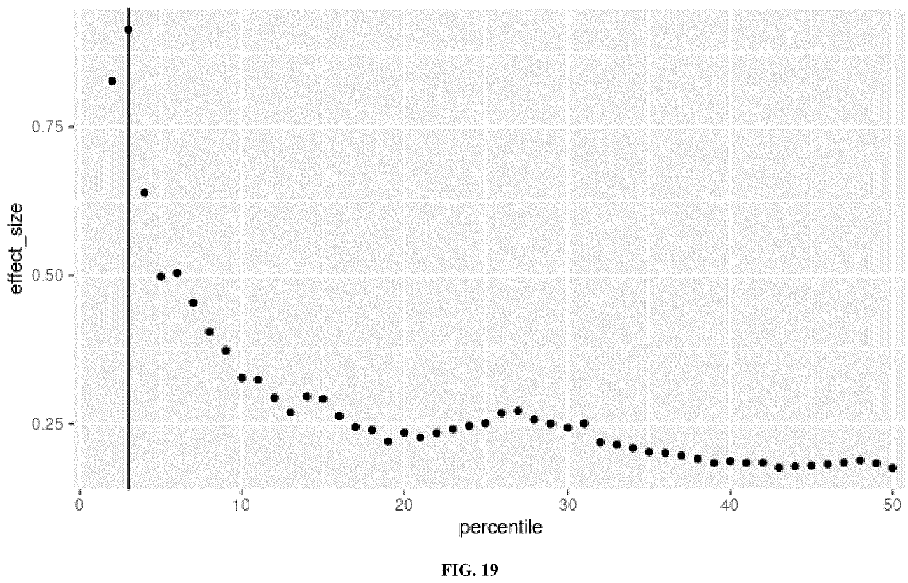

Samples were referred to as Small for Gestational Age (SGA) when baby weight percentiles were less than or equal to 3, 5, 7.5, and 10, with corresponding percentiles of SGA in our case population of 2.1%, 3.6%, 5.9%, and 8.3%. To maximize the ratio of growth restricted vs. constitutionally small babies, and to capture the most severe cases of fetal growth restriction, we confined fetal growth restriction cases to fall within the 3rd baby weight percentile (SGA3), yielding 58 cases of fetal growth restriction, annotated in the following as SGA3. This 3% cut-off was based on medical knowledge and later confirmed by effect size analysis of leading model features for predicting different baby weight percentile at birth shown in FIG. 19. Effect size (Cohen's d) peaks at baby weight percentile cut off of 3% for SGA (vertical line). The peak at 3% baby weight cut-off is consistent with medical practice and classification of severe growth restriction.

First, DESeq2 differential gene expression analyses were performed for 58 SGA3 cases. Table 19 shows 33 genes identified by differential expression to correlate with the risk of developing fetal restriction growth SGA3 with p-value<0.05, after correcting p-values for multiple comparisons using Holm's method.

TABLE 19
A set of 30 top differentially expressed genes that were
identified to correlate to the risk of developing fetal
growth restriction SGA3 after correcting p-values
for multiple comparisons using Holm's method

| Index | Gene | P-value | P-value adj. |
|---|---|---|---|
| 1 | RHBDD1 | 1.89E−08 | 0.00024835 |
| 2 | PTK2 | 1.14E−07 | 0.00149364 |
| 3 | SIRPA | 1.27E−07 | 0.00166907 |
| 4 | HCK | 1.33E−07 | 0.00174586 |
| 5 | GCA | 1.52E−07 | 0.00199759 |
| 6 | NLRC4 | 1.57E−07 | 0.00206846 |
| 7 | CEBPD | 2.34E−07 | 0.00308074 |
| 8 | ZC3H12C | 2.56E−07 | 0.00336159 |
| 9 | B4GALT5 | 5.75E−07 | 0.00756281 |
| 10 | ST3GAL3 | 5.99E−07 | 0.00787907 |
| 11 | ZFPM2 | 6.92E−07 | 0.00909116 |
| 12 | PTP4A2P2 | 7.59E−07 | 0.00997976 |
| 13 | PLCB4 | 7.79E−07 | 0.01024436 |
| 14 | COL24A1 | 9.34E−07 | 0.01227991 |
| 15 | IMPDH1 | 1.08E−06 | 0.01414758 |
| 16 | RAB3D | 1.13E−06 | 0.01481151 |
| 17 | SLA | 1.22E−06 | 0.01601702 |
| 18 | GRAP2 | 1.30E−06 | 0.01714503 |
| 19 | LRRC4 | 1.59E−06 | 0.02088838 |
| 20 | RGS6 | 1.76E−06 | 0.02314638 |
| 21 | ALOX5 | 1.86E−06 | 0.0244105 |
| 22 | NCKAP1 | 1.88E−06 | 0.02471005 |
| 23 | GTF2IP4 | 2.29E−06 | 0.03004466 |
| 24 | PDGFRA | 2.83E−06 | 0.03718659 |
| 25 | PFKFB4 | 2.86E−06 | 0.03754445 |
| 26 | LPCAT2 | 2.86E−06 | 0.03760202 |
| 27 | RASGRP4 | 2.99E−06 | 0.03929802 |
| 28 | HSPA1A | 3.01E−06 | 0.03948474 |
| 29 | PRAM1 | 3.11E−06 | 0.04083438 |
| 30 | ARHGAP6 | 3.48E−06 | 0.0456316 |
| 31 | ASAP2 | 3.52E−06 | 0.04617731 |
| 32 | CDC14B | 3.52E−06 | 0.04621062 |
| 33 | RP2 | 3.62E−06 | 0.04755193 |

To identify genetic predictors of fetal growth restriction, cross-validated feature discovery with training and testing splits at a 3:4 ratio was performed with 60 resamples. Within each resample, features were selected according to the Sure Independence Screening (SIS) method developed by Fan et al. (Fan J, Lv J. (2008). Sure Independence Screening (SIS) for Ultrahigh Dimensional Feature Space. Journal of the Royal Statistical Society B, 70(5), 849-911. doi:10.1111/j.1467-9868. 2008.00674.x., which is incorporated by reference herein in its entirety), implemented in the R programming language library SIS by Saldana et al. (Saldana, D. F., & Feng, Y. (2018). SIS: An R Package for Sure Independence Screening in Ultrahigh-Dimensional Statistical Models. Journal of Statistical Software, 83(2), 1-25., which is incorporated by reference herein in its entirety).

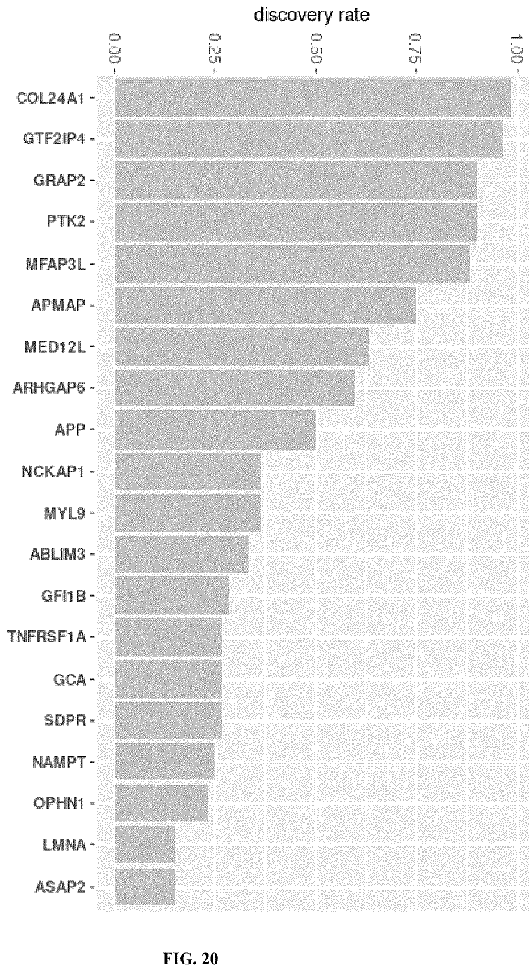

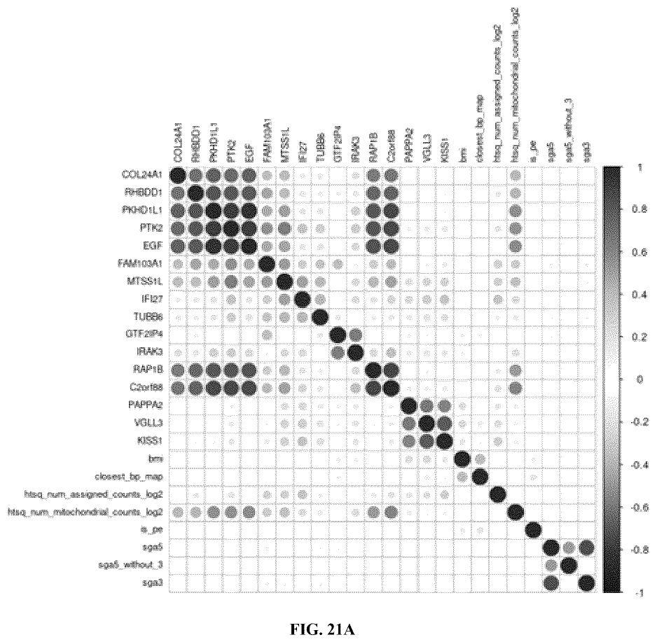

In the SIS correlation learning framework feature selection consists of a two-stage process. During the first stage, features are ranked by the magnitude of their marginal regression coefficients with the target variable and the first n/log(n) features are selected to reduce the dimensionality of the feature space to below the number of samples, n. The second stage involves further reduction of the feature space with the statistically principled L-1 norm regularization method LASSO, implemented in the R-package “glmnet” (Friedman J, Tibshirani R, Hastie T (2010). “Regularization Paths for Generalized Linear Models via Coordinate Descent.” Journal of Statistical Software, 33(1), 1-22., which is incorporated by reference herein in its entirety). The parameters for the LASSO procedure were determined via the penalized likelihood method, Akaike Information Criterion (AIC). After applying SIS in each of the 60 resampled training sets the relative frequency of recurring discovered features were calculated and shown in FIG. 20. Table 20 shows the 20 top predictive genes with discovery rates >15% for SGA3 during training discovery.

TABLE 20
Set of 20 top predictive genes for SGA3
with discovery rates >15% from
60 fold cross-validated feature discovery.

| Index | Gene | Discovery rate (%) |
|---|---|---|
| 1 | COL24A1 | 97.5 |
| 2 | GTF2IP4 | 95.0 |
| 3 | GRAP2 | 90.0 |
| 4 | PTK2 | 90.0 |
| 5 | MFAP3L | 88.3 |
| 6 | APMAP | 75.0 |
| 7 | MED12L | 63.3 |
| 8 | ARHGAP6 | 60.0 |
| 9 | APP | 50.0 |
| 10 | NCKAP1 | 36.7 |
| 11 | MYL9 | 36.7 |
| 12 | ABLIM3 | 30.0 |
| 13 | GFI1B | 28.3 |
| 14 | TNFRSF1A | 26.7 |
| 15 | GCA | 26.7 |
| 16 | SPDR | 26.7 |
| 17 | NAMPT | 25.0 |
| 18 | OPHN1 | 23.3 |
| 19 | LMNA | 15.0 |
| 20 | ASAP2 | 15.0 |

Repeating cross-validated feature discovery for different data transformations (log 2, log 2CPM, and down sampled data) and for various minimum assigned count thresholds, we found consistently four genes (GTF2IP4, COL24A1, RHBDD1, and PTK2) to be predictive for SGA3.

GTF2IP4, COL24A1, and PTK2 may be associated with placental and fetal development (Chatzizacharias N A, Kouraklis G P, Theocharis S E. The role of focal adhesion kinase in early development. Histol Histopathol. 2010 August; 25(8):1039-55. doi: 10.14670/HH-25.1039. PMID: 20552554., which is incorporated by reference herein in its entirety).

### Example 11: Methodologies to Identify the Gene Correction to Improve Fetal Growth Restriction Modeling

Using systems and methods of the present disclosure, to control technical and biological variation not related to fetal growth restriction, an algorithm was developed to identify genes correlated with the SGA3 model predictors but not with the condition SGA3 itself.

The algorithm selects those genes according to a two-step correlation ranking. First, a list is created where the genes were ranked in decreasing order by the magnitude of their correlations with the model predictors. Next, a list is generated of genes ranked in increasing order by the magnitude of their correlations with the outcome variable SGA3. Those genes which rank high in both lists, e.g., which are uncorrelated (adjusted p-value (Holm) >0.2) with the outcome variable but highly correlated (Pearson correlation coefficient >0.5) with the SGA3 model predictors, are then selected as potential gene correction features.

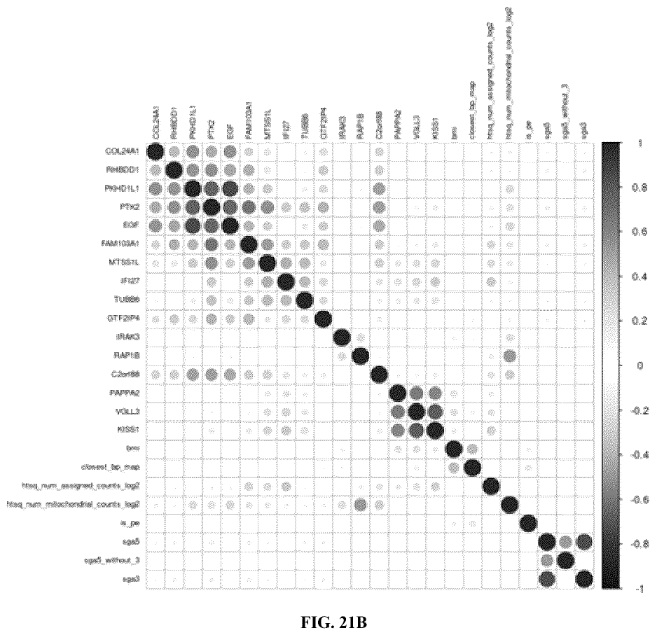

Within this framework, we selected two correction genes, RAP1B and IRAK3, where RAP1B is correlated with the COL24A1 group of model features and IRAK3 is correlated with the model predictor GTF2IP4 shown in FIGS. 21A-21B. The first group contains most model features, whereas the second group consists only of GTF2IP4. The correction genes, RAP1B and IRAK3, are correlated with the first and second group, respectively. The gene corrections increase correlations between model predictors and SGA3 and de-correlate model features from the number of assigned counts and the number of mitochondrial counts. Model predictors are clustered within two groups. We find that both correction genes increase the correlations of model predictors with SGA3 while removing technical and biological variation with the number of assigned counts and the number of mitochondrial counts.

### Example 12: Early Prediction of Preeclampsia and Molecular Subtypes of Preeclampsia from a 2,971-Person Prospective Trial Using cfRNA Profiling

Using systems and methods of the present disclosure, early differentially expressed molecular markers of preeclampsia were identified in maternal blood samples using a prospective cohort of 2,971 pregnant females described in Example 8. All samples were processed using a unified experimental and computational NGS pipeline for cell-free (cfRNA) sequencing to generate gene counts. Twin pregnancies were excluded.

Plasma samples were collected in the gestational age range of 17.5-22 weeks and sequencing must yield a minimum number of assigned gene counts. QC metrics were applied to require that the number of genes with CPM above 0 to be less than 24,000. Assigned gene counts for each sample was downsampled to a target count (e.g. 5.5 million) by a binomial procedure and Log 2 transformed. Gene candidates included genes significant to pregnancy with a mean CPM greater than 1.

First, modeling of preterm preeclampsia was performed for cases when participants developed preeclampsia during pregnancy, and newborn babies were delivered by medically-indicated intervention before 37 weeks of gestational age.

Preterm preeclampsia samples were defined as maternal subjects with preeclampsia who delivered before 37 weeks (<37 weeks) (n=91), and all else (including maternal subjects with preeclampsia who delivered at term >=37 weeks) were treated as controls (n=2259). Fifty 70/30 train/test splits were sampled from the dataset. Within each fold, feature discovery and model training were performed on the train set (64 cases/1581 controls) and model evaluation was performed on the test set (27 cases/678 controls).

During feature discovery, differential expression based on the Mann-Whitney U test for preterm preeclampsia was performed on linear model residuals corrected for fetal fraction based on VGLL3 expression levels. An L2 logistic model was trained to predict preterm preeclampsia based on the top 5 differentially expressed features combined with fetal fraction correction feature VGLL3 and technical corrections for assay stability (the number of assigned counts, the environmental temperature a sample is exposed to, delta qPCR ct values for housekeeping gene ACTB in extraction or ERCC spike-ins in library, and the percent of deduplication in the pipeline).

Features were scaled by median and interquartile range parameterized on the training data to be robust to outliers. 43 gene features discovered across the fifty train/test folds are shown in Table 21. When the models also include clinical factors BMI, diastolic blood pressure prior to the time of collection, chronic hypertension status, and prior PE status, 98% of the folds exceed performance of 70% sensitivity and 70% specificity within the test folds. When considering clinical features BMI, diastolic blood pressure prior to the time of collection, chronic hypertension status, and prior preeclampsia status or prior preterm birth status, 98% or 100% of the folds exceed performance of 70% sensitivity and 70% specificity within the test folds, respectively.

TABLE 21
Set of 43 genes identified for preterm preeclampsia
(<37 weeks) with VGLL3 correction across 50 train/test
splits ordered by detected frequency across the folds.

| | | % folds |
|---|---|---|
| Index | Gene | identified |
| 1 | PAPPA2 | 100 |
| 2 | LILRB5 | 72 |
| 3 | KISS1 | 46 |
| 4 | CD163 | 38 |
| 5 | ADAM12 | 34 |
| 6 | MDGA1 | 26 |
| 7 | SH3PXD2A | 20 |
| 8 | VSIG4 | 14 |
| 9 | FBXW11 | 12 |
| 10 | TCHH | 12 |
| 11 | PCBP2 | 12 |
| 12 | ZNF595 | 8 |
| 13 | IPO11 | 8 |
| 14 | TBCEL | 6 |
| 15 | CYP11A1 | 6 |
| 16 | ZNF768 | 6 |
| 17 | FBXO9 | 6 |
| 18 | THRB | 6 |
| 19 | EFHD1 | 6 |
| 20 | KCNT2 | 4 |
| 21 | XAGE2 | 4 |
| 22 | CCNI | 4 |
| 23 | SNX6 | 4 |
| 24 | TTC30A | 4 |
| 25 | CD209 | 4 |
| 26 | GYPB | 4 |
| 27 | PPP1R14A | 2 |
| 28 | CGA | 2 |
| 29 | MAFK | 2 |
| 30 | CLEC4C | 2 |
| 31 | MBD3 | 2 |
| 32 | FLT3 | 2 |
| 33 | ATP6V1E1 | 2 |
| 34 | TPCN2 | 2 |
| 35 | HIST2H2BF | 2 |
| 36 | DAGLB | 2 |
| 37 | YBX1P2 | 2 |
| 38 | SMG1P6 | 2 |
| 39 | LMTK2 | 2 |
| 40 | NEK11 | 2 |
| 41 | TCEAL9 | 2 |
| 42 | GPNMB | 2 |
| 43 | ARG2 | 2 |

Second, modeling was performed on a combined group: 1) preterm preeclampsia with delivery <37 weeks, and 2) term preeclampsia with severe features delivered after >−=37 weeks.

In order to increase the power for discovering features for severe preeclampsia, moms with preeclampsia with severe features who managed to deliver at term (>=37 weeks) were combined with moms with preterm preeclampsia (delivered early due to severity of disease) to form the case group (n=170) and were compared to 2206 controls.

The controls included maternal subjects with term preeclampsia without severe features. One hundred 70/30 train/test splits were sampled from the dataset. Within each fold, feature discovery and model training was performed on the train set (119 cases/1544 controls) and model evaluation was performed on the test set (51 cases/662 controls).

During feature discovery, differential expression based on the Mann-Whitney U test for preterm preeclampsia and term preeclampsia with severe features was performed on linear model residuals corrected for BMI and fetal fraction based on VGLL3 expression levels. 81 gene features discovered across the one hundred train/test folds are shown in Table 22 across the full dataset for term preeclampsia with severe features.

TABLE 22
Set of 81 genes identified for severe preeclampsia
(preterm preeclampsia (<37 weeks) plus term
preeclampsia with severe features (>=37 weeks))
based on BMI and VGLL3 across discovery
of 100 train/test splits sorted by detected
frequency across the folds.

| | | % folds |
|---|---|---|
| Index | gene | identified |
| 1 | PAPPA2 | 100 |
| 2 | NSRP1 | 47 |
| 3 | FBXW11 | 31 |
| 4 | EBI3 | 26 |
| 5 | DYNC112 | 24 |
| 6 | SREK1 | 23 |
| 7 | ADAM12 | 22 |
| 8 | CD163 | 19 |
| 9 | SH3PXD2A | 17 |
| 10 | MUC3A | 14 |
| 11 | MMRN2 | 14 |
| 12 | EFHD1 | 11 |
| 13 | FAT4 | 11 |
| 14 | CENPB | 10 |
| 15 | KISS1 | 8 |
| 16 | RNASE1 | 8 |
| 17 | SPTLC3 | 8 |
| 18 | TPM3P9 | 7 |
| 19 | ABCB4 | 7 |
| 20 | LILRB5 | 5 |
| 21 | GPNMB | 5 |
| 22 | ZNF768 | 5 |
| 23 | NFE2L1 | 3 |
| 24 | CGA | 3 |
| 25 | EIF5B | 3 |
| 26 | DMWD | 3 |
| 27 | GTF2H2B | 2 |
| 28 | ZNF358 | 2 |
| 29 | ZSCAN18 | 2 |
| 30 | RHOC | 2 |
| 31 | ERCC6L | 2 |
| 32 | CGNL1 | 2 |
| 33 | KLF11 | 2 |
| 34 | RAB30 | 2 |
| 35 | IL18RAP | 2 |
| 36 | GGA2 | 2 |
| 37 | BIRC3 | 2 |
| 38 | PHC3 | 1 |
| 39 | SLC25A12 | 1 |
| 40 | PCBP1 | 1 |
| 41 | RASGEF1B | 1 |
| 42 | ATF6 | 1 |
| 43 | STAG3L4 | 1 |
| 44 | SMC3 | 1 |
| 45 | INPP5F | 1 |
| 46 | TRIM65 | 1 |
| 47 | BTLA | 1 |
| 48 | TTC28 | 1 |
| 49 | MACROD2 | 1 |
| 50 | UPF3A | 1 |
| 51 | RYR2 | 1 |
| 52 | LYRM9 | 1 |
| 53 | IGIP | 1 |
| 54 | TCEAL4 | 1 |
| 55 | LPGAT1 | 1 |
| 56 | PCBP2 | 1 |
| 57 | TRAF3IP1 | 1 |
| 58 | CAPN6 | 1 |
| 59 | SPIB | 1 |
| 60 | BRAP | 1 |
| 61 | N4BP3 | 1 |
| 62 | LMTK2 | 1 |
| 63 | TCHH | 1 |
| 64 | NCOA3 | 1 |
| 65 | JUN | 1 |
| 66 | TCEAL9 | 1 |
| 67 | TNFSF4 | 1 |
| 68 | EBF1 | 1 |
| 69 | HIST2H2BF | 1 |
| 70 | CD34 | 1 |
| 71 | CXCR5 | 1 |
| 72 | CCDC57 | 1 |
| 73 | CACYBPP2 | 1 |
| 74 | PLAG1 | 1 |
| 75 | ZEB1 | 1 |
| 76 | PTPRG | 1 |
| 77 | DYNC1I2P1 | 1 |
| 78 | SPRY1 | 1 |
| 79 | NEK11 | 1 |
| 80 | CD209 | 1 |
| 81 | VSIG4 | 1 |

Third, discovery for preterm preeclampsia without major risk factors.

Preeclampsia without major risk factors is defined as moms with preeclampsia without any high risk factors as defined by the U.S. Preventive Services Task Force (USPSTF) guidelines and recommendations, such as no history of preeclampsia, chronic hypertension, or pregestational diabetes, etc.

Samples were excluded if they were positive for any of the high risk factors regardless of delivery gestational age or preeclampsia status. Preterm preeclampsia without major risk factors are moms with preeclampsia without major risk factors who delivered before 37 weeks (n=49). Preeclampsia moms without major risk factors who delivered at term >=37 weeks were treated as controls and combined with moms without preeclampsia and also without major risk factors (n=1967). One hundred 80/20 train/test splits were sampled from the dataset. Within each fold, feature discovery and model training was performed on the train set (39 cases/1573 controls) and model evaluation was performed on the test set (10 cases/394 controls).

During feature discovery, differential expression based on the Mann-Whitney U test for preterm preeclampsia without major risk factors was performed on linear model residuals corrected for fetal fraction based on VGLL3 expression levels. An L2 logistic model was trained to predict preterm preeclampsia without major risk factors based on the top 5 differentially expressed features combined with fetal fraction correction feature VGLL3 and technical corrections for assay stability (the number of assigned counts, the environmental temperature a sample is exposed to, delta qPCR ct values for housekeeping genes ACTB in extraction, and the percent of deduplication in the pipeline). Features were scaled by median and interquartile range parameterized by controls in training to be robust to outliers.

50 gene features discovered across the hundred train/test folds are shown in Table 23. When the models include clinical factors body mass index (BMI) and diastolic blood pressure prior to the time of collection, 82% of the folds exceed performance of 70% sensitivity and 70% specificity within the test folds, where median test AUC is 0.84 [95% CI: 0.75-0.94].

TABLE 23
Set of 50 genes identified for preterm preeclampsia (<37
weeks) without major risk factors with VGLL3 across 100
train/test splits ordered by detected frequency across the folds.

| Index | Gene | % folds identified |
|---|---|---|
| 1 | PAPPA2 | 100 |
| 2 | ADAM12 | 58 |
| 3 | KISS1 | 44 |
| 4 | EBI3 | 43 |
| 5 | FBXW11 | 39 |
| 6 | CCNI | 31 |
| 7 | HIST2H3D | 20 |
| 8 | CACYBPP2 | 16 |
| 9 | FABP1 | 15 |
| 10 | RHAG | 14 |
| 11 | N4BP3 | 12 |
| 12 | PPP1R2P3 | 11 |
| 13 | TBCEL | 10 |
| 14 | HIST1H3I | 9 |
| 15 | PCBP2 | 6 |
| 16 | HIST1H3D | 5 |
| 17 | ARSK | 5 |
| 18 | PLAC4 | 4 |
| 19 | ACTG1 | 4 |
| 20 | RAB30 | 4 |
| 21 | CCDC61 | 3 |
| 22 | KCNN3 | 3 |
| 23 | NCOA3 | 3 |
| 24 | GPATCH2L | 3 |
| 25 | TFDP1 | 3 |
| 26 | HIST1H1E | 2 |
| 27 | ARPP19 | 2 |
| 28 | GYPB | 2 |
| 29 | ALB | 2 |
| 30 | CYP11A1 | 2 |
| 31 | SOX12 | 2 |
| 32 | ZNF595 | 2 |
| 33 | YWHAEP5 | 2 |
| 34 | DYNC112 | 2 |
| 35 | TCEAL9 | 2 |
| 36 | HIST1H4B | 1 |
| 37 | SMAD6 | 1 |
| 38 | CGA | 1 |
| 39 | HIST1H1C | 1 |
| 40 | SLC2A1 | 1 |
| 41 | PCBP1 | 1 |
| 42 | TM9SF1 | 1 |
| 43 | MTMR12 | 1 |
| 44 | POLR2J3 | 1 |
| 45 | CSRNP1 | 1 |
| 46 | GFPT1 | 1 |
| 47 | DNM1L | 1 |
| 48 | PHACTR1 | 1 |
| 49 | EVI2B | 1 |
| 50 | NUDT6 | 1 |

### Example 13: Methodologies to Correct Differential Gene Analyses to Improve Modeling for Developing a Robust Preeclampsia Classifier

### 1) Correction for Technical Variation, Such as Computationally Stabilizing Assay Variation.

Achieve assay variation stability computationally by applying technical corrections to gene counts by accounting for measurable metrics, such as those descriptive of the sample, laboratory processes, sequencing, or bioinformatics pipeline. Target metrics were identified based on minimizing the number of genes significant to pregnancy that are differentially expressed across a train and test dataset split, where the expected number of genes that are differentially expressed is zero in the absence of systematic sources of variance. A set of technical corrections were defined based on the largest reduction in the number of differential genes across the dataset split with consideration for boosting performance with each additional metric incorporated to minimize overfitting. The residuals from a linear model estimating gene counts from the technical corrections for each gene of interest were compared by differential expression to evaluate performance. Metrics were identified by looking for differential performance across the train/test split and measuring orthogonality by correlation. An example of identified metrics include the number of assigned counts, the environmental temperature a sample is exposed to, delta qPCR cycle threshold (Ct) values for housekeeping genes such as ACTB in extraction or ERCC spike-ins in library, or the percent of deduplication in the pipeline. Correcting by these 5 technical corrections can decrease variance across genes significant to pregnancy with a mean CPM >1 by 10%.

### 2) Correction for Sample-to-Sample Variation, Such as Normalizing or Correcting Using Genes (e.g., Identified by Negative Binomial Dispersion Residuals).

Gene based corrections were identified to control for sample-to-sample variation. Count based discrete distributions like the negative binomial apply well to modeling gene counts measured by sequencing of cell-free RNA. The negative binomial quantifies count variance as a quadratic relationship to the count mean parameterized by the dispersion parameter:

$$variance = mean + dispersion parameter * mean2$$

Twenty technical repeats were developed from plasma combined from pregnant moms. In this dataset, the dispersion relationship was fit to placenta genes significant to pregnancy with a minimal Log 2 fold change amplitude of 1.5 after calculating the residuals with respect to the percent deduplicated. Genes were sorted based on descending magnitude of their residual to the fit dispersion relationship relative to their means to generate a list of highly variable genes. Correction genes were selected by evaluating the compression in variance of the significant pregnancy genes with mean CPM >1 based on the residuals with respect to these variable genes while maintaining the explained variance between the original and corrected variance (r2>0.9). Correction genes KISS1, XAGE2, PSG2, ADAM12, PSG9, and CYP19A1 in combination resulted in boosted preterm preeclampsia L2 logistic classifier performance, where the test AUC significantly improved (p<0.01) when compared to the models developed without correction by these genes as well as when compared to a correction by a random set of 6 genes. Performance across 50 train/test splits of median train size of 57 cases/1422 controls versus a test set size of 25 cases/608 controls had a test performance of AUC=0.76 [95% CI: 0.67-0.84] for genes discovered with the corrections in the presence of technical corrections for the number of assigned counts, the environmental temperature a sample is exposed to, delta qPCR Ct values for housekeeping genes such as ACTB in extraction or ERCC spike-ins in library, and the percent of deduplication in the pipeline.

Preterm preeclampsia effect size can be further boosted by applying this technique to identify variable genes significant to preterm preeclampsia by combining variable genes identified from technical repeat with those identified from samples without preeclampsia (1865 controls) and from samples with preterm preeclampsia (82 cases) by fitting gene counts in each group corrected for the environmental temperature a sample is exposed to, delta qPCR Ct values for housekeeping genes such as ACTB in extraction or ERCC spike-ins in library, and the percent of deduplication in the pipeline with the negative binomial dispersion relationship. Variable genes were determined to be significant to preterm preeclampsia based on the Akaike Information Criterion (AIC). Gene corrections identified through this method are KISS1, ADAM12, CSH1, HSD17B1, and CSH2. The effect size for preterm preeclampsia for PAPPA2, KRT7, and KRT8 residuals after correcting for these 5 genes were found to be significantly larger than the effect size for preterm preeclampsia resulting from correcting with 5 random genes (50 sets compared). Genes were corrected for the total assigned counts, the environmental temperature a sample is exposed to, delta qPCR Ct values for housekeeping genes such as ACTB in extraction or ERCC spike-ins in library, and the percent of deduplication in the pipeline.

### 3) Correcting for Collinearity Effects (e.g., Due to Correlated and Biochemically Related Genes)

Correction genes can be identified to reduce noise in a target feature used in a classifier by searching for genes that are correlated with the feature of interest and also not correlated to the label predicted by the classifier. For a given feature, its Log 2 CPM gene expression levels are fitted against a gene search list (e.g. genes with means greater than 20 counts) and sorted in descending order based on the coefficient of determination based off a robust linear regression using Huber's T for M estimation. The top gene is identified based on the maximal coefficient of determination for those with a non-significant p-value by Mann-Whitney U test for the label. For preeclampsia, SVEP1 was identified as a correction gene for PAPPA2 (1498 samples with assigned counts of at least 2 million collected in 17-22 weeks gestational age and adjusted for cohort effects) with a non-significant p-value for preeclampsia (p>0.05, 156 preeclampsia cases/1307 controls). Correcting PAPPA2 by BMI with & without SVEP1 shows that its differential expression for preeclampsia improved by an order of magnitude (156 preeclampsia cases/1307 controls), where the correction reduces variance across samples.

Correction genes can also be genes in the same biochemical pathway as features important to the pregnancy complication to power the discovery of additional gene features for a classification model. For example, PAPPA2 is a strong signal for preterm preeclampsia (<37 weeks), and IGFBP5 is a known substrate of the PAPPA2 enzyme where PAPPA2 cleaves IGFBP5 to release sequestered IGF for signaling. Significant pregnancy genes with CPM greater than 1 when corrected by IGFBP5, alongside a fetal fraction correction gene VGLL3, increases the signal for preterm preeclampsia, such as for genes like PAPPA2 and SH3PXD2A, and also boosts the power for genes that previously were not significant to become significant for use as a classification feature, e.g., TCHH, VSIG4, RNASE1, and TCEAL9 (p<0.05) presented in Table 24.

TABLE 24
Set of 10 genes identified after additional connection
IGFBP5 for preterm preeclampsia (<37 weeks) following
VGLL3 and IGFBP5 correction on Log2 transformed
gene counts based on the Mann-Whitney
U test, and the p-values corrected for multiple
tests are shown when VGLL3 and IGFBP5 are
used to correct versus correction by VGLL3 alone.

| | | 1. p-value, | p-value, |
|---|---|---|---|
| Index | Gene | VGLL3 + IGFBP5 | VGLL3 |
| 1 | PAPPA2 | 6.00E−10 | 1.62E−09 |
| 2 | LILRB5 | 4.25E−03 | 3.92E−03 |
| 3 | SH3PXD2A | 0.01 | 0.10 |
| 4 | CD163 | 0.01 | 0.01 |
| 5 | KISS1 | 0.01 | 0.01 |
| 6 | ADAM12 | 0.04 | 0.03 |
| 7 | TCHH | 0.04 | 0.06 |
| 8 | VSIG4 | 0.04 | 0.06 |
| 9 | RNASE1 | 0.04 | 0.24 |
| 10 | TCEAL9 | 0.04 | 0.17 |

Correction factors can also be applied as ratios with respect to the target gene, e.g. PAPPA2 and CGA, as an alternative treatment to linear correction, to achieve similar improvement to signal-to-noise when building a classifier.

### 4) Fetal Fraction Correction Based on Gene Correlations to Male Fetus Signals (VGLL3).

Cell-free RNA from the fetus varies between samples. To control for fetal fraction variation across samples, correction genes are identified by searching for significant placenta genes that are correlated to a known fetal signature. Since genes on Chr Y are specific to males, the expression levels for genes on Chr Y in pregnancies with male fetuses estimate the fetal signatures in cell-free RNA. Correlations against the total gene expression level across Chr Y genes in Log 2 counts downsampled to 3M assigned counts per sample were performed with placenta genes significant to pregnancy to identify placental genes that can correct for the variation in fetal fraction (1507 samples). Fits are sorted in descending order by the coefficient of determination calculated with a robust linear regression using Huber's T for M estimation. The top gene that was correlated to the sum across Chr Y genes in samples from pregnancies with male fetuses was VGLL3 (1507 samples). Cell free RNA expression levels summed across the placenta genes when corrected by VGLL3 show no differential levels within pregnancies between male or female fetuses (p>0.05), as well as when looking across baby sex within preterm preeclampsia cases or within controls by Mann-Whitney U test for Log 2 transformed counts (p>0.05).

### Example 14: Clinical Intervention Care Pathway to Improve Pre-Term Birth Outcomes Based at Least in Part on Molecular Subtype Prediction Test Performed in Second Trimester

Using systems and methods of the present disclosure, a clinical intervention care plan algorithm is developed to improve spontaneous pre-term birth (sPTB) outcomes based at least in part the results of molecular subtype predictive tests that may be performed on a pregnant subject in the second trimester.

Currently, there is no reliable pre-term birth test available for an asymptomatic general population without prior preterm history, and a majority of pregnancies are followed to routine prenatal care pathway. Moreover, there is no ability to predict spontaneous pre-term birth cases before 34 weeks of gestation age. The earlier in pregnancy a baby is born, the more likely they may be to have health problems. Babies born before 34 weeks of pregnancy are most likely to have health problems, but babies born between 34 and 37 weeks of pregnancy are also at increased risk of having health problems related to premature birth. For example, some premature babies need to spend time in a hospital's newborn intensive care unit (NICU).

Common main health complications for babies born at 34 weeks of gestation include difficulty with lung capacity and breathing. The earlier a baby is born, the greater the likelihood of bleeding in the brain. Therefore, there remains an urgent unmet need for a molecular test capable of identifying pregnant subjects with increased risk of spontaneous preterm birth before 34 weeks gestational age, which allows the opportunity for early clinical action and to use a variety of potentially useful clinical interventions to prevent or delay delivery.

A clinical intervention care pathway algorithm for pregnant subjects with increased risk of early preterm birth (before 34 weeks) may include three specific phases, including the delay or prevention of onset of labor, starting early serial monitoring for signs of labor onset, and the proactive or prophylactic administration of corticosteroids in the event of threatening labor.

In the first phase care pathway, more aggressive clinical intervention can be used to delay spontaneous labor. Various labor delay treatments or procedures may include use low-dose aspirin, consideration of cerclage placement, pessary placement, or treatment with progesterone. Additional testing that may be of potential benefit include nucleic acid amplification testing (in addition to routine screening for sexually transmitted infections) for the identification of bacterial vaginosis², Mycoplasma genitalium³, and Ureaplasma parvum³ and urealyticum⁴ which may identify underlying infections that have been associated with sPTB.

In the second phase care pathway, early active monitoring of labor signs may allow for clinical preparation for preterm delivery. The monitoring may include use of serial cervical length assessment with ultrasound or serial testing with fetal fibronectin.

In the third phase care pathway, there may be an increased likelihood of administration of corticosteroids for fetal benefit in the event of threatened preterm labor, to allow faster fetal maturation of the unborn baby to reduce the risk of serious complications or the newborn dying.

### REFERENCES

1. Groom K M, David A L. The role of aspirin, heparin, and other interventions in the prevention and treatment of fetal growth restriction. Am J Obstet Gynecol. 2018 February; 218(2S):S829-S840. doi: 10.1016/j.ajog.2017.11.565. Epub 2017 Dec. 8. PMID: 29229321 is incorporated by reference herein in its entirety.
2. Leitich H, Brunbauer M, Bodner-Adler B, Kaider A, Egarter C, Husslein P. Antibiotic treatment of bacterial vaginosis in pregnancy: a meta-analysis. Am J Obstet Gynecol. 2003 March; 188(3):752-8. doi: 10.1067/mob.2003.167. PMID: 12634652 is incorporated by reference herein in its entirety.
3. Donders G G G, Ruban K, Bellen G, Petricevic L. Mycoplasma/Ureaplasma infection in pregnancy: to screen or not to screen. J Perinat Med. 2017 Jul. 26; 45(5):505-515. doi: 10.1515/jpm-2016-0111. PMID: 28099135 is incorporated by reference herein in its entirety.
4. Raynes-Greenow C H, Roberts C L, Bell J C, Peat B, Gilbert G L. Antibiotics for ureaplasma in the vagina in pregnancy. Cochrane Database Syst Rev. 2004; (1):CD003767. doi: 10.1002/14651858.CD003767.pub2. Update in: Cochrane Database Syst Rev. 2011; (9):CD003767. PMID: 14974036 is incorporated by reference herein in its entirety.

### Example 15: Clinical Intervention Care Pathway to Improve Preeclampsia (PE) Outcomes Based at Least in Part on Molecular Subtype Prediction Test Performed in Second Trimester

Using systems and methods of the present disclosure, a clinical intervention care plan algorithm is developed to improve preeclampsia outcomes based at least in part on results of molecular subtype predictive tests that may be performed on a pregnant subject in the second trimester.

Currently, there is no preeclampsia test available for an asymptomatic general population without prior history of hypertension or prior preeclampsia, and a majority of pregnancies are followed to routine prenatal care pathway. Also, preventive management of preeclampsia may be only provided to high risk pregnant subjects, which may be determined based on, for example, prior history, demographic, socioeconomic, or clinical factors. For example, recommended preeclampsia management in high-risk individuals is summarized in www.ajog.org/article/S0002-9378(23)00260-0/fulltext, which is incorporated by reference herein in its entirety, and may include:

Increased monitoring: Home blood pressure monitoring may be associated with up to a 50% reduction in preeclampsia.¹ Baseline laboratory evaluation is also recommended, and sleep study is suggested in individuals with symptoms suggestive of obstructive sleep apnea, which may be associated with an increased risk of hypertensive disorders of pregnancy. (www.ajog.org/article/S0002-9378(23)00260-0/fulltext, which is incorporated by reference herein in its entirety).

Low-dose aspirin: Low-dose aspirin may be associated with a reduction in preeclampsia ranging from 8-43%.²⁻⁵

Vitamin D supplementation: Vitamin D supplementation may be associated with a 52% reduction in preeclampsia.⁶

Calcium supplementation if insufficient intake: Calcium supplementation may be associated with a 55% reduction in preeclampsia in a low-calcium population.⁷

Exercise: Regular exercise may be associated with a 46% reduction in preeclampsia.⁸

Mediterranean diet: Mediterranean diet may be associated with a 28% reduction in preeclampsia.⁹

The clinical management for the management of individuals at high risk of preeclampsia may also include maintenance of blood pressure at <140/90 for individuals with chronic hypertension, which may be associated with a 21% reduction in preeclampsia.¹⁰

Therefore, there remains an urgent unmet need for a predictive molecular subtype test capable of identifying pregnant subjects with increased risk of different type of preeclampsia, which allows the opportunity for early clinical action and to use a variety of potentially useful clinical interventions to prevent and manage preeclampsia effectively for previously unaddressed pregnancies without prior history or clinical factors, and reducing the overtreatment of pregnancies from the high-risk group.

A highly predictive molecular subtype test may be most impactful for identification of pregnant subjects who are at risk of developing preterm preeclampsia with most sever features causing medically-indicated deliveries <37 weeks.

A clinical intervention care pathway algorithm for pregnant subjects with increased risk of early preterm preeclampsia or preeclampsia with severe features may include three specific phases, including adding those pregnancies to current guidelines to prevent or reduce the probability of developing preeclampsia, starting early serial monitoring for signs of preeclampsia, and the proactive or prophylactic administration of therapeutics to manage blood pressure or improve kidney or liver functions.

In the first phase care pathway, preventive or prophylactic clinical intervention can be used to delay onset of preeclampsia. Various preventive or prophylactic delay treatments or procedures may include use of low-dose aspirin, vitamin D, and calcium supplementations, change in lifestyle (e.g., sleep, diet, and exercise), and medicated management of blood pressure for pregnant subjects with chronic hypertension.

In the second phase care pathway, early active monitoring of blood pressure, and serial testing for kidney and liver functions may allow for clinical detection of early onset of preeclampsia.

In the third phase care pathway, there may be an increased likelihood of administration of therapeutics, including but not limited to metformin¹¹, statins¹², proton pump inhibitors,¹³ sulfasalazine,¹³ eculizumab¹³, and molecular-target strategies (e.g., monoclonal antibodies targeting tumor necrosis factor alpha, placental growth factor, and short interfering RNA.)¹³. Any of these therapeutic options may provide clinical benefit for certain molecular subtypes of preeclampsia but not for others (e.g., for the prevention of preeclampsia with severe features at <37 weeks, without associated reduction in term preeclampsia observed).

Finally, in maternal subjects at high risk of any of the above subtypes of preeclampsia, a risk-reducing induction of labor at 39-40 weeks estimated gestational age may be performed to further reduce the incidence of preeclampsia in pregnant subjects who reach that gestational age.

### REFERENCES

1. Kalafat E, Benlioglu C, Thilaganathan B, Khalil A. Home blood pressure monitoring in the antenatal and postpartum period: A systematic review meta-analysis. Pregnancy Hypertens. 2020 January; 19:44-51. doi: 10.1016/j.preghy.2019.12.001. Epub 2020 Jan. 2. PMID: 31901652 is incorporated by reference herein in its entirety.
2. Henderson J T, Vesco K K, Senger C A, Thomas R G, Redmond N. Aspirin Use to Prevent Preeclampsia and Related Morbidity and Mortality: Updated Evidence Report and Systematic Review for the US Preventive Services Task Force. JAMA. 2021 Sep. 28; 326(12):1192-1206. doi: 10.1001/jama.2021.8551. PMID: 34581730 is incorporated by reference herein in its entirety.
3. LeFevre M L; U.S. Preventive Services Task Force. Low-dose aspirin use for the prevention of morbidity and mortality from preeclampsia: U.S. Preventive Services Task Force recommendation statement. Ann Intern Med. 2014 Dec. 2; 161(11):819-26. doi: 10.7326/M14-1884. PMID: 25200125 is incorporated by reference herein in its entirety.
4. Askie L M, Duley L, Henderson-Smart D J, Stewart L A; PARIS Collaborative Group. Antiplatelet agents for prevention of pre-eclampsia: a meta-analysis of individual patient data. Lancet. 2007 May 26; 369(9575):1791-1798. doi: 10.1016/50140-6736(07)60712-0. PMID: 17512048 is incorporated by reference herein in its entirety.
5. Duley L, Henderson-Smart D J, Meher S, King J F. Antiplatelet agents for preventing pre-eclampsia and its complications. Cochrane Database Syst Rev. 2007 Apr. 18; (2):CD004659. doi: 10.1002/14651858.CD004659.pub2. Update in: Cochrane Database Syst Rev. 2019 Oct. 30; 2019(10): PMID: 17443552 is incorporated by reference herein in its entirety.
6. Palacios C, Kostiuk L K, Peña-Rosas J P. Vitamin D supplementation for women during pregnancy. Cochrane Database Syst Rev. 2019 Jul. 26; 7(7):CD008873. doi: 10.1002/14651858.CD008873.pub4. PMID: 31348529; PMCID: PMC6659840 is incorporated by reference herein in its entirety.
7. Hofmeyr G J, Lawrie T A, Atallah A N, Torloni M R. Calcium supplementation during pregnancy for preventing hypertensive disorders and related problems. Cochrane Database Syst Rev. 2018 Oct. 1; 10(10):CD001059. doi: 10.1002/14651858.CD001059.pub5. PMID: 30277579; PMCID: PMC6517256 is incorporated by reference herein in its entirety.
8. Danielli M, Gillies C, Thomas R C, Melford S E, Baker P N, Yates T, Khunti K, Tan B K. Effects of Supervised Exercise on the Development of Hypertensive Disorders of Pregnancy: A Systematic Review and Meta-Analysis. J Clin Med. 2022 Feb. 1; 11(3):793. doi: 10.3390/jcm11030793. PMID: 35160245; PMCID: PMC8836524 is incorporated by reference herein in its entirety.
9. Makarem N, Chau K, Miller E C, Gyamfi-Bannerman C, Tous I, Booker W, Catov J M, Haas D M, Grobman W A, Levine L D, McNeil R, Bairey Merz C N, Reddy U, Wapner R J, Wong M S, Bello N A. Association of a Mediterranean Diet Pattern With Adverse Pregnancy Outcomes Among US Women. JAMA Netw Open. 2022 Dec. 1; 5(12):e2248165. Doi: 10.1001/jamanetworkopen.2022.48165. PMID: 36547978; PMCID: PMC9857221 is incorporated by reference herein in its entirety.
10. Tita A T, Szychowski J M, Boggess K, Dugoff L, Sibai B, Lawrence K, Hughes B L, Bell J, Aagaard K, Edwards R K, Gibson K, Haas D M, Plante L, Metz T, Casey B, Esplin S, Longo S, Hoffman M, Saade G R, Hoppe K K, Foroutan J, Tuuli M, Owens M Y, Simhan H N, Frey H, Rosen T, Palatnik A, Baker S, August P, Reddy U M, Kinzler W, Su E, Krishna I, Nguyen N, Norton M E, Skupski D, El-Sayed Y Y, Ogunyemi D, Galis Z S, Harper L, Ambalavanan N, Geller N L, Oparil S, Cutter G R, Andrews W W; Chronic Hypertension and Pregnancy (CHAP) Trial Consortium. Treatment for Mild Chronic Hypertension during Pregnancy. N Engl J Med. 2022 May 12; 386(19):1781-1792. doi: 10.1056/NEJMoa2201295. Epub 2022 Apr. 2. PMID: 35363951; PMCID: PMC9575330 is incorporated by reference herein in its entirety.
11. Romero R, Erez O, Huttemann M, Maymon E, Panaitescu B, Conde-Agudelo A, Pacora P, Yoon B H, Grossman L I. Metformin, the aspirin of the 21st century: its role in gestational diabetes mellitus, prevention of preeclampsia and cancer, and the promotion of longevity. Am J Obstet Gynecol. 2017 September; 217(3):282-302. doi: 10.1016/j.ajog.2017.06.003. Epub 2017 Jun. 12. PMID: 28619690; PMCID: PMC6084482 is incorporated by reference herein in its entirety.
12. Smith D D, Costantine M M. The role of statins in the prevention of preeclampsia. Am J Obstet Gynecol. 2022 February; 226(2S):S1171-S1181. doi: 10.1016/j.ajog.2020.08.040. Epub 2020 Aug. 17. PMID: 32818477; PMCID: PMC8237152 is incorporated by reference herein in its entirety.
13. Tong S, Kaitu'u-Lino T J, Hastie R, Brownfoot F, Cluver C, Hannan N. Pravastatin, proton-pump inhibitors, metformin, micronutrients, and biologics: new horizons for the prevention or treatment of preeclampsia. Am J Obstet Gynecol. 2022 February; 226(2S):S1157-S1170. doi: 10.1016/j.ajog.2020.09.014. Epub 2020 Sep. 16. PMID: 32946849 is incorporated by reference herein in its entirety.

### Example 16: Clinical Intervention Care Pathway to Improve Gestational Diabetes Mellitus (GDM) Outcomes Based at Least in Part on Molecular Subtype Prediction Test Performed in Second Trimester

Using systems and methods of the present disclosure, a clinical intervention care plan algorithm is developed to improve GDM outcomes based at least in part on results of molecular subtypes predictive tests that may be performed on a pregnant subject in the second trimester.

Currently, there is no gestational diabetes mellitus test available for an asymptomatic general population in early second trimester, and a majority of pregnancies are followed to routine prenatal care pathway, which may include a diagnostic oral glucose tolerance test at 24-28 weeks of gestational age.

Currently, clinical standard of care may include screening pregnant subjects at high risk based on clinical factors (e.g., history of GDM in a prior pregnancy, obesity, etc.) for type 2 diabetes mellitus (DM) at the first pregnancy visit. In those pregnant subjects who screen negative on their initial testing, and in all a priori average-risk pregnant subjects, screening for GDM is performed between 24-28 weeks estimated gestational age. Screening may be performed via an oral glucose challenge, in which the pregnant subject drinks a beverage with a prespecified carbohydrate load, and her blood sugar is measured thereafter. There are a substantial number of pregnant subjects who are unable tolerate the oral glucose challenge and for whom an alternative may be useful¹. The glucose drink may be associated with a variety of gastrointestinal complaints in the general pregnant population¹ and dumping syndrome in those who have had a Roux-en-Y gastric bypass procedure².

With current standard of care, pregnant subjects who screen positive for gestational diabetes on their one-hour test may require a follow-up test with a three-hour diagnostic test, at which time gestational diabetes is diagnosed.³ After diagnosis, pregnant subjects are advised to implement a carbohydrate-controlled diet and glucose monitoring throughout pregnancy. However, there is currently no way to predict which pregnant subjects may develop either A1GDM or A2GDM subtypes, and the diagnosis may be made based on the blood sugar trend after appropriate nutritional and lifestyle interventions have been implemented.

Moreover, early onset of hyperglycemia may start significantly earlier in pregnancy and may develop into impaired glucose in tolerance detectable by OGTT test at 24-28 weeks of gestational age. At that time, for many pregnant subjects, the insulin injection to control hyperglycemia is the only treatment option.

Therefore, there remains an urgent unmet need for a predictive molecular subtype test which may be performed early in pregnancy (18-20 weeks) to assess the risk of developing GDM, which allows the opportunity for early clinical action, such as developing specific clinical intervention care plans early.

A clinical intervention care pathway algorithm for pregnant subjects with increased risk of development of either GDMA1 or GDMA2 subtypes may include three specific phases. First, starting earlier on healthy diet and lifestyle may more likely benefit the GDMA1 population, and may delay the start of medication use for GDMA2 group. Second, the implementation of a predictive test in the second trimester of pregnancy to predict GDMA1 and GDMA2 may simplify the glucose tolerance test procedure. The GDMA1 population may forego the one-hour test and/or the 3-hour diagnostic test, and the GDMA2 population may proceed directly to a 3 hour diagnostic test. In the third phase, the GDMA2 group may consider starting treatment earlier initiation of insulin, oral hypoglycemic agents, or other therapeutics to mitigate and control hyperglycemia to further reduce fetal risks for macrosomia, neonatal hypoglycemia, hyperbilirubinemia, and respiratory distress syndrome, and the maternal subject's risk for developing type 2 DM later in life.

### REFERENCES

1. Agarwal M M, Punnose J, Dhatt G S. Gestational diabetes: problems associated with the oral glucose tolerance test. Diabetes Res Clin Pract. 2004 January; 63(1):73-4. doi: 10.1016/j.diabres.2003.08.005. PMID: 14693415 is incorporated by reference herein in its entirety.
2. Guelinckx I, Devlieger R, Vansant G. Reproductive outcome after bariatric surgery: a critical review. Hum Reprod Update. 2009 March-April; 15(2):189-201. doi: 10.1093/humupd/dmn057. Epub 2009 Jan. 8. PMID: 19136457 is incorporated by reference herein in its entirety.
3. ACOG Practice Bulletin No. 190: Gestational Diabetes Mellitus. Obstet Gynecol. 2018 February; 131(2):e49-e64. doi: 10.1097/AOG.0000000000002501. PMID: 29370047 is incorporated by reference herein in its entirety.

### Example 17: Clinical Intervention Care Pathway to Improve Intrauterine Growth Restriction or Fetal Growth Restriction Outcomes Based at Least in Part on Molecular Subtype Prediction Test Performed in Second Trimester

Using systems and methods of the present disclosure, a clinical intervention care plan algorithm is developed to improve outcomes for pregnant subjects identified to be at high risk of having a fetus with intrauterine growth restriction or fetal growth restriction resulting in a small-for-gestational-age (SGA) newborn, based at least in part on the results of molecular predictive tests that may be performed on a pregnant subject in the second trimester.

Intrauterine growth restriction is a leading cause of perinatal morbidity and mortality¹, and poses a management challenge to obstetricians. The current clinical standard of care fails to identify the majority of SGA infants², which is notable because SGA fetuses not identified prior to delivery have a four-fold increased risk of adverse fetal outcomes compared to those who are identified and monitored antenatally.³

Therefore, there remains an urgent unmet need for a predictive molecular subtype test capable of identifying pregnant subjects with increased risk of SGA3 or SGA 3-10 early in pregnancy (18-20 weeks), who may benefit from a specific intervention care path to improve outcome for those pregnancies.

A clinical intervention care pathway algorithm for pregnant subjects with increased risk of fetal growth restriction may include three specific phases.

At present, most average-risk pregnancies do not receive further assessment of fetal growth after the 20-week anatomy survey. In the first phase of clinical care pathway phase for early assessment and surveillance, a population of pregnant subjects positive for high-risk SGA3 or SGA3-10 may benefit from increased surveillance in pregnancy, including interventions such as serial ultrasound assessment of growth, amniotic fluid index, assessment of fetal wellbeing (e.g., non-stress tests, biophysical profiles), and umbilical artery Doppler assessment. These assessments may be performed in pregnancy when fetal growth restriction has been identified, however they may not improve outcomes in an unselected population. Therefore, the availability of a non-invasive molecular test to predict SGA infants may allow the selection of the appropriate risk group of pregnant subjects to receive additional surveillance, thereby translating to improved detection of fetal growth restriction and improved fetal as well as maternal clinical outcomes.

In addition, based on the molecular subtypes identified, a subgroup of infants are identified who are constitutionally small, rather than being pathologically growth-restricted. In this case, a more relaxed fetal surveillance program may be appropriate.

In the second phase, there may be an increased likelihood of the use of therapeutic treatment to prevent or reduce intrauterine growth restriction outcome. Such therapeutic treatments may include phosphodiesterase-5 enzyme inhibitors⁴, statins⁵,low-dose aspirin⁶, heparin and low-molecular-weight heparin,⁷ micro RNAs⁷, vascular endothelial growth factor gene therapy⁷, proton pump inhibitors⁷, melatonin⁷, creatine⁷, and N-acetylcysteine⁷, among others. With the availability of a molecular test to predict SGA at various severity levels, the appropriate groups of pregnant may be selected for these and other potential therapeutic agents in the future.

In the third phase, to reduce the incidence of still birth or cerebral palsy, medically-indicated birth may be considered with delivery at 38 weeks.

### REFERENCES

1. Resnik R. Intrauterine growth restriction. Obstet Gynecol. 2002 March; 99(3):490-6. doi: 10.1016/s0029-7844(01)01780-x. PMID: 11864679 is incorporated by reference herein in its entirety.
2. Zafman K B, Cudjoe E, Levine L D, Srinivas S K, Schwartz N. Undetected Fetal Growth Restriction During the Coronavirus Disease 2019 (COVID-19) Pandemic. Obstet Gynecol. 2023 Feb. 1; 141(2):414-417. doi: 10.1097/AOG.0000000000005052. Epub 2023 Jan. 4. PMID: 36649315 is incorporated by reference herein in its entirety.
3. Lindqvist P G, Molin J. Does antenatal identification of small-for-gestational age fetuses significantly improve their outcome? Ultrasound Obstet Gynecol. 2005 March; 25(3):258-64. doi: 10.1002/uog.1806. PMID: 15717289 is incorporated by reference herein in its entirety.
4. Ferreira RDDS, Negrini R, Bernardo W M, Simões R, Piato S. The effects of sildenafil in maternal and fetal outcomes in pregnancy: A systematic review and meta-analysis. PLoS One. 2019 Jul. 24; 14(7):e0219732. doi: 10.1371/journal.pone.0219732. PMID: 31339910; PMCID: PMC6655684 is incorporated by reference herein in its entirety.
5. Mendoza M, Ferrer-Oliveras R, Bonacina E, Garcia-Manau P, Rodo C, Carreras E, Alijotas-Reig J. Evaluating the Effect of Pravastatin in Early-Onset Fetal Growth Restriction: A Nonrandomized and Historically Controlled Pilot Study. Am J Perinatol. 2021 December; 38(14):1472-1479. doi: 10.1055/s-0040-1713651. Epub 2020 Jul. 2. PMID: 32615618 is incorporated by reference herein in its entirety.
6. Roberge S, Nicolaides K, Demers S, Hyett J, Chaillet N, Bujold E. The role of aspirin dose on the prevention of preeclampsia and fetal growth restriction: systematic review and meta-analysis. Am J Obstet Gynecol. 2017 February; 216(2):110-120.e6. doi: 10.1016/j.ajog.2016.09.076. Epub 2016 Sep. 15. PMID: 27640943 is incorporated by reference herein in its entirety.
7. Groom K M, David A L. The role of aspirin, heparin, and other interventions in the prevention and treatment of fetal growth restriction. Am J Obstet Gynecol. 2018 February; 218(2S):S829-S840. doi: 10.1016/j.ajog.2017.11.565. Epub 2017 Dec. 8. PMID: 29229321 is incorporated by reference herein in its entirety.

### Example 18: Methods for Defining Molecular Subtypes of Preeclampsia and Prediction Performance within the Molecularly Defined Group

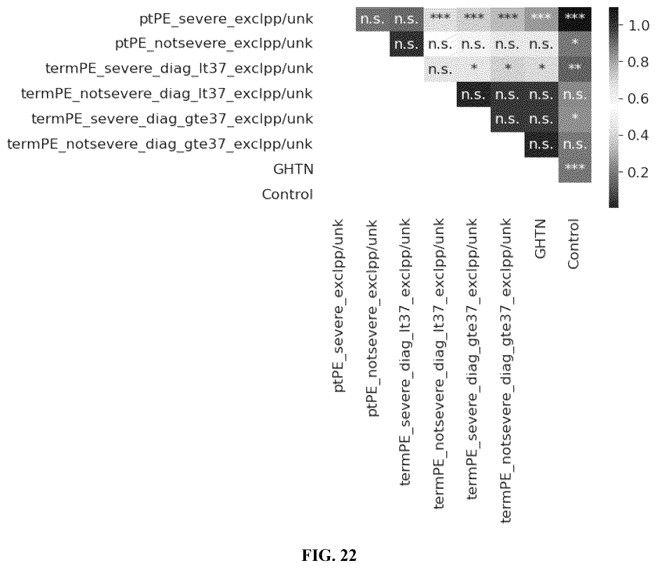

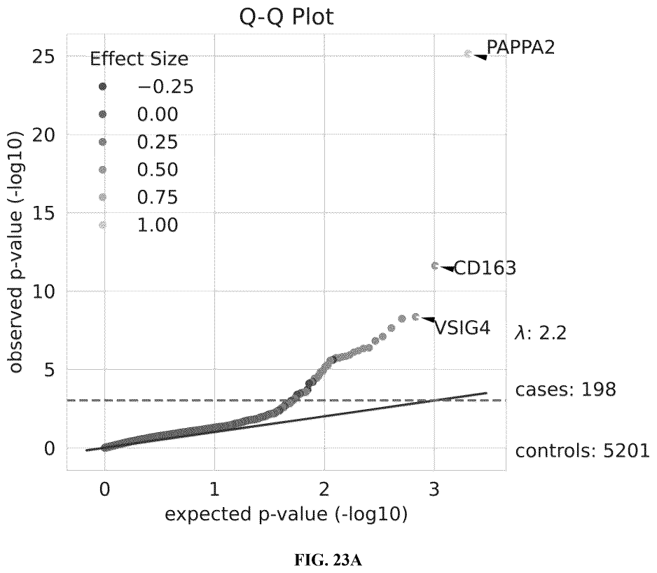

Preterm preeclampsia may be driven by PAPPA2, and a molecular subtype of preeclampsia can be defined based on gene expression profile. PAPPA2 was observed to be the most differentially expressed gene in a prospective cohort of 5,399 individuals described in Example 5. Overlaying PAPPA2 expression with a split of the data along 3 dimensions—diagnosis time before or after 37 weeks, delivery time before or after 37 weeks, and presence or absence of severe features—shows PAPPA2 signal clustering with preterm preeclampsia diagnosed before 37 weeks, delivered before 37 weeks and with severe features if diagnosed before 37 weeks (FIG. 22), this group is referred to as PAPPA2+ preeclampsia.

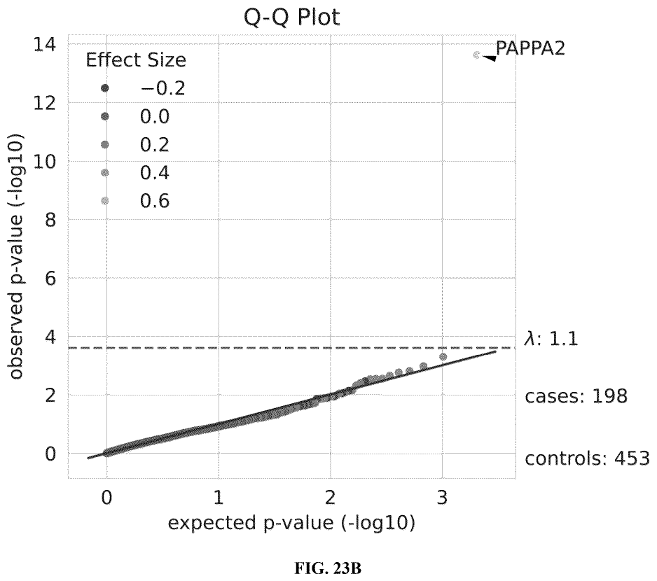

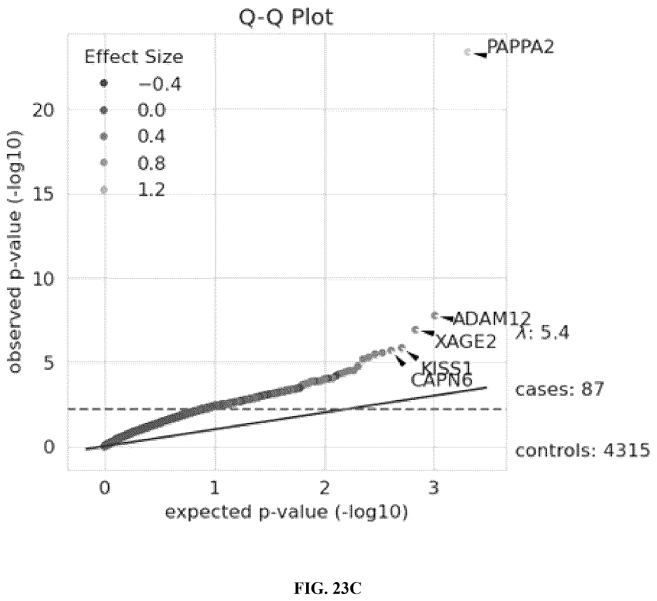

Within the PAPPA2+ preeclampsia group, several genes were observed to be differentially expressed, as shown in FIG. 23A. Table 25 shows the top 20 differentially expressed genes in PAPPA2+ samples, highlighting raw and multiple testing p-values by Mann-Whitney U and effect size measured by Cohen's d. The results also show that within individuals diagnosed with preeclampsia, PAPPA2 is the only gene among the set that is differentially expressed between PAPPA2+ and PAPPA2− preeclampsia (FIG. 23B).

TABLE 25
Set of top 20 differentially expressed
genes in PAPPA2+ samples.

| Gene | p-value | Adjusted p-value | Effect size |
|---|---|---|---|
| PAPPA2 | 7.40E−26 | 1.51E−22 | 1.00617678 |
| CD163 | 2.46E−12 | 2.51E−09 | 0.53036464 |
| VSIG4 | 4.43E−09 | 2.96E−06 | 0.47982131 |
| KISS1 | 5.81E−09 | 2.96E−06 | 0.43363383 |
| TCHH | 2.30E−08 | 9.35E−06 | 0.46812431 |
| LILRB5 | 7.93E−08 | 2.69E−05 | 0.4294584 |
| XAGE2 | 1.49E−07 | 4.33E−05 | 0.37179956 |
| AXL | 4.14E−07 | 0.00010409 | 0.40433318 |
| IGSF21 | 4.60E−07 | 0.00010409 | 0.39400427 |
| SLCO2B1 | 6.46E−07 | 0.00013147 | 0.42359824 |
| CD209 | 8.01E−07 | 0.0001483 | 0.40260435 |
| CIQC | 1.22E−06 | 0.00020615 | 0.3684506 |
| CAPN6 | 1.50E−06 | 0.00023521 | 0.40746687 |
| STAB1 | 1.65E−06 | 0.0002402 | 0.37082141 |
| EBI3 | 1.91E−06 | 0.00024801 | 0.3721501 |
| PSG11 | 1.95E−06 | 0.00024801 | 0.349332 |
| GYPB | 2.57E−06 | 0.00030733 | −0.3586697 |
| ADAM12 | 2.86E−06 | 0.00032303 | 0.3165463 |
| MRC1 | 5.51E−06 | 0.00059088 | 0.36656933 |
| FCGBP | 7.12E−06 | 0.00072457 | 0.36991638 |
| GPR34 | 1.26E−05 | 0.00122467 | 0.33073272 |

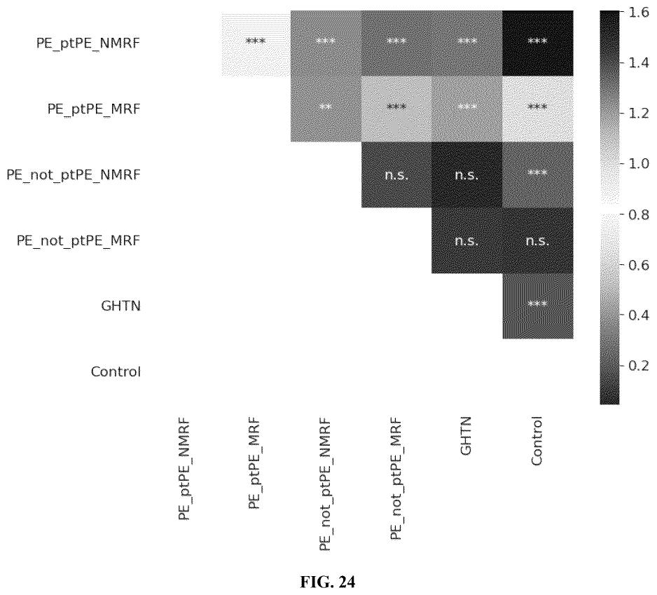

Further, the signal for preeclampsia was also dependent on presence or absence of preexisting major risk factor for preeclampsia, as defined by the USPSTF. In preeclampsia cases with PAPPA2+ preeclampsia the signal, as measured by Cohen's d effect size, was significantly larger in individuals without preexisting major risk factors (FIG. 24). Samples with major risk factors were still significantly differentiated from PAPPA2− preeclampsia, gestational hypertension, or individuals with any hypertensive disorder of pregnancy (FIG. 24).

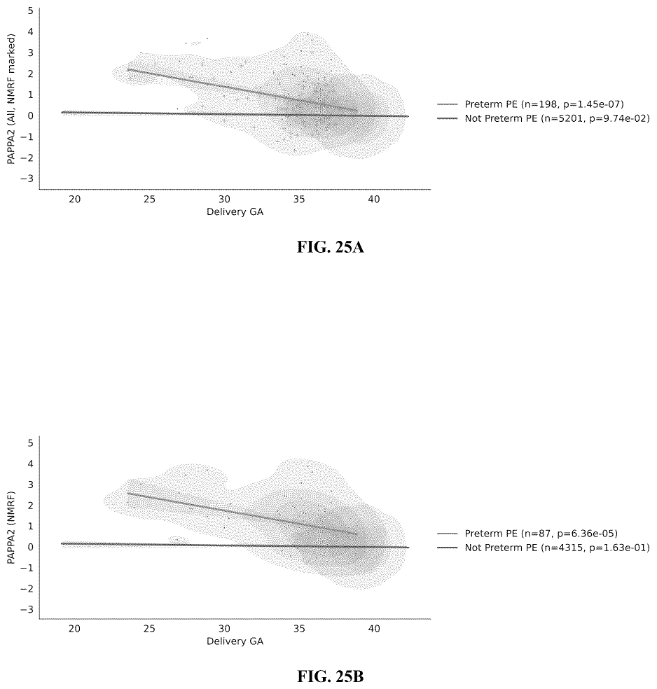

The results indicate a dose-dependence between PAPPA2 signal and gestational age at delivery, such that individuals with PAPPA2+ preeclampsia are more likely to deliver earlier as their PAPPA2 value increased, as was observed for all samples (FIG. 25A) and for samples without major risk factors (FIG. 25B). Significance is evaluated by the slope on the linear fit, which was p=1.45e-7 and p=6.36e-5 for all PAPPA2+ samples and for PAPPA2+ samples without major risk factors, respectively. Average separation was best for individuals without major risk factors, as seen in FIG. 24.

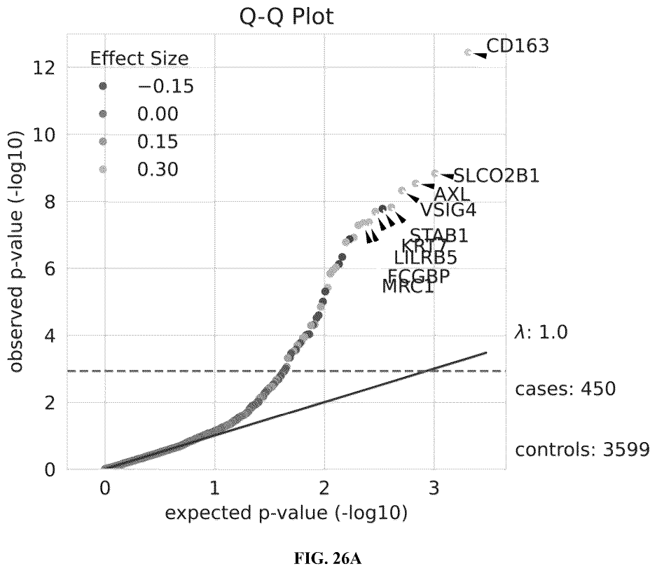

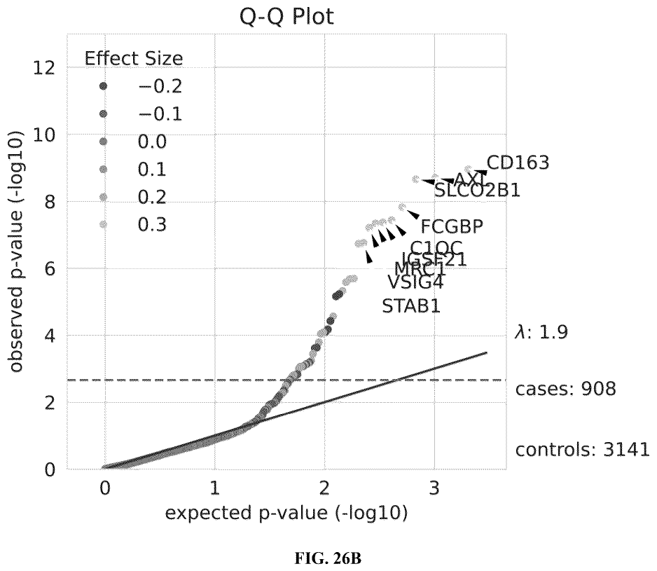

For samples not in the top quartile of PAPPA2 expression, several immune genes were observed in the set of top 20 most differentially expressed genes, which applies whether the group of all preeclampsia cases was considered (FIG. 26A) or the group of all hypertensive disorders of pregnancy cases was considered (FIG. 26B). The top differentially expressed genes in both of those groups was CD163. A full list of the top 20 differentially expressed genes in the bottom three quartiles of PAPPA2 expression is provided in Table 26 for the group of all preeclampsia cases and Table 27 for the group of all hypertensive disorders of pregnancy cases.

TABLE 26
Differentially expressed genes from the bottom three
quartiles of PAPPA2 expression for all preeclampsia cases.

| Gene | p-value | Adjusted p-value | Effect size |
|---|---|---|---|
| CD163 | 1.06E−13 | 2.15E−10 | 0.3892958 |
| SLCO2B1 | 6.10E−10 | 6.21E−07 | 0.32283568 |
| AXL | 1.25E−09 | 8.47E−07 | 0.31290607 |
| VSIG4 | 2.07E−09 | 1.05E−06 | 0.31017291 |
| STAB1 | 7.07E−09 | 2.69E−06 | 0.29822054 |
| KRT7 | 7.92E−09 | 2.69E−06 | −0.282642 |
| LILRB5 | 9.71E−09 | 2.82E−06 | 0.30200022 |
| FCGBP | 2.04E−08 | 4.91E−06 | 0.29850122 |
| MRC1 | 2.17E−08 | 4.91E−06 | 0.29311568 |
| IGSF21 | 2.54E−08 | 5.17E−06 | 0.29312645 |
| CIQC | 6.10E−08 | 1.13E−05 | 0.28292079 |
| KRT8 | 6.97E−08 | 1.18E−05 | −0.2743366 |
| PAPPA2 | 8.60E−08 | 1.35E−05 | 0.27806583 |
| FBLN1 | 2.49E−07 | 3.61E−05 | −0.2346565 |
| KRT18 | 4.14E−07 | 5.62E−05 | −0.2465164 |
| CD209 | 5.26E−07 | 6.69E−05 | 0.2579084 |
| TCHH | 6.37E−07 | 7.63E−05 | 0.27006128 |
| EBI3 | 8.21E−07 | 9.29E−05 | 0.2573759 |
| LYVE1 | 2.23E−06 | 0.00023931 | 0.29265494 |
| GYPB | 2.96E−06 | 0.00030174 | −0.24205 |
| MAOA | 6.10E−06 | 0.00059114 | −0.2309443 |

TABLE 27
Differentially expressed genes from the bottom
three quartiles of PAPPA2 expression for all
hypertensive disorders of pregnancy cases.

| Gene | p-value | Adjusted p-value | Effect size |
|---|---|---|---|
| CD163 | 5.18E−18 | 1.06E−14 | 0.33269797 |
| AXL | 1.63E−17 | 1.32E−14 | 0.31666584 |
| SLCO2B1 | 1.95E−17 | 1.32E−14 | 0.31644813 |
| FCGBP | 8.12E−16 | 4.13E−13 | 0.3176084 |
| C1QC | 4.55E−15 | 1.85E−12 | 0.2945852 |
| IGSF21 | 6.15E−15 | 2.03E−12 | 0.29320946 |
| MRC1 | 6.97E−15 | 2.03E−12 | 0.29495223 |
| VSIG4 | 1.23E−14 | 3.14E−12 | 0.29890577 |
| STAB1 | 9.25E−14 | 2.09E−11 | 0.27832253 |
| LILRB5 | 1.04E−13 | 2.12E−11 | 0.28853037 |
| LYVE1 | 1.06E−11 | 1.90E−09 | 0.27391528 |
| CD209 | 1.12E−11 | 1.90E−09 | 0.25662571 |
| C1QB | 1.71E−11 | 2.68E−09 | 0.24878198 |
| TCHH | 5.43E−11 | 7.89E−09 | 0.25388609 |
| GYPB | 8.46E−11 | 1.15E−08 | −0.2404548 |
| KRT7 | 1.13E−10 | 1.43E−08 | −0.2492409 |
| ITPR1 | 1.54E−09 | 1.84E−07 | 0.22462216 |
| MOSPD1 | 2.96E−09 | 3.34E−07 | −0.2242338 |
| MAOA | 9.03E−09 | 9.68E−07 | −0.2198233 |
| KRT8 | 1.26E−08 | 1.28E−06 | −0.2144989 |
| PAPPA2 | 1.32E−08 | 1.28E−06 | 0.22052029 |

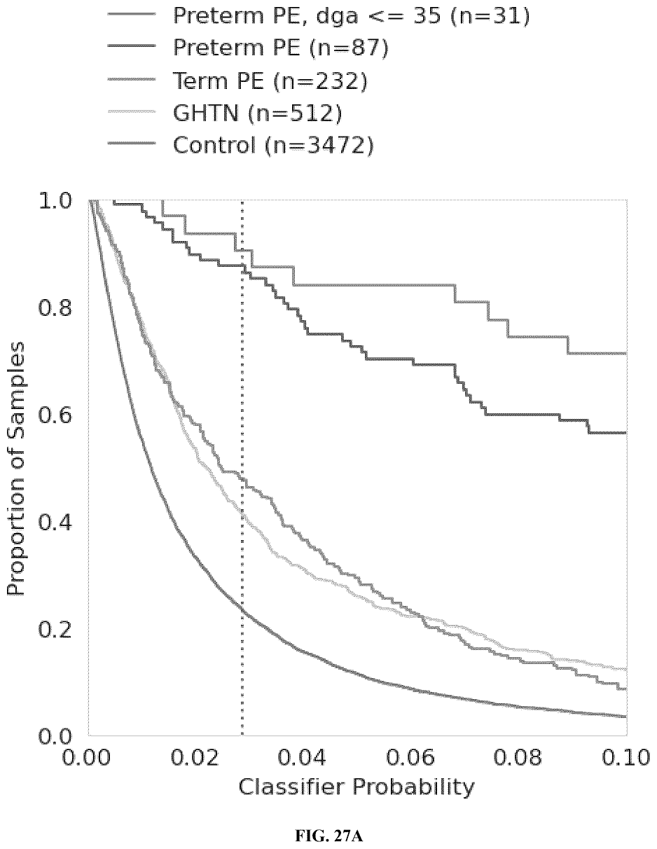

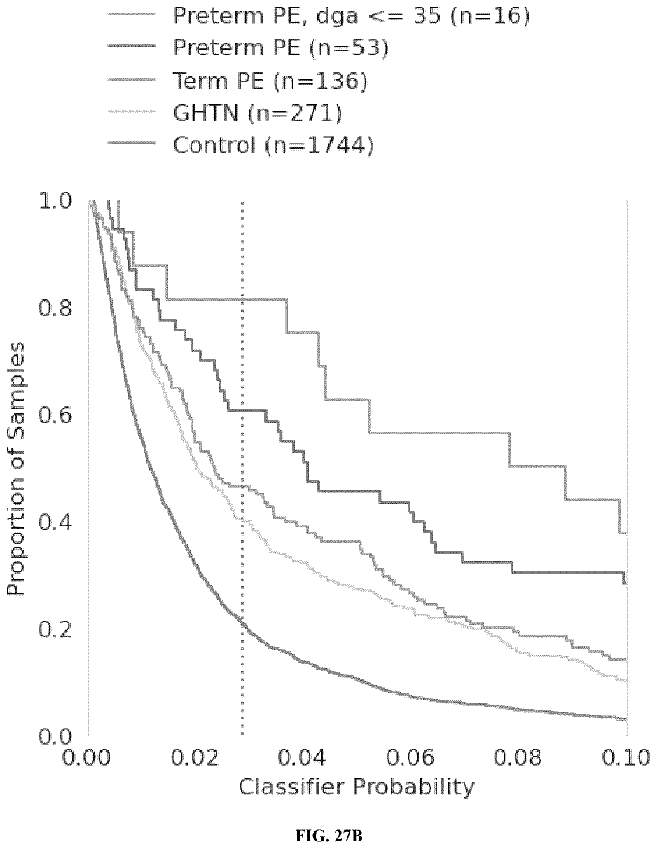

A classifier was developed to predict preterm preeclampsia (PAPPA2+ preeclampsia), which can also detect other forms of preeclampsia and hypertensive disorder of pregnancy, albeit with slightly lower sensitivity. Separation of the different severity levels was observed in the Kaplan-Meier plot for training (FIG. 27A) and the validation set (FIG. 27B). In both plots, the ranking of severity is persevered with PAPPA2+PE delivered <=35 weeks at the top, followed by all PAPPA2+PE, then PAPPA2− PE and GHTN, all of which were clearly separated from individuals not diagnosed with a hypertensive disorder of pregnancy (control cases).

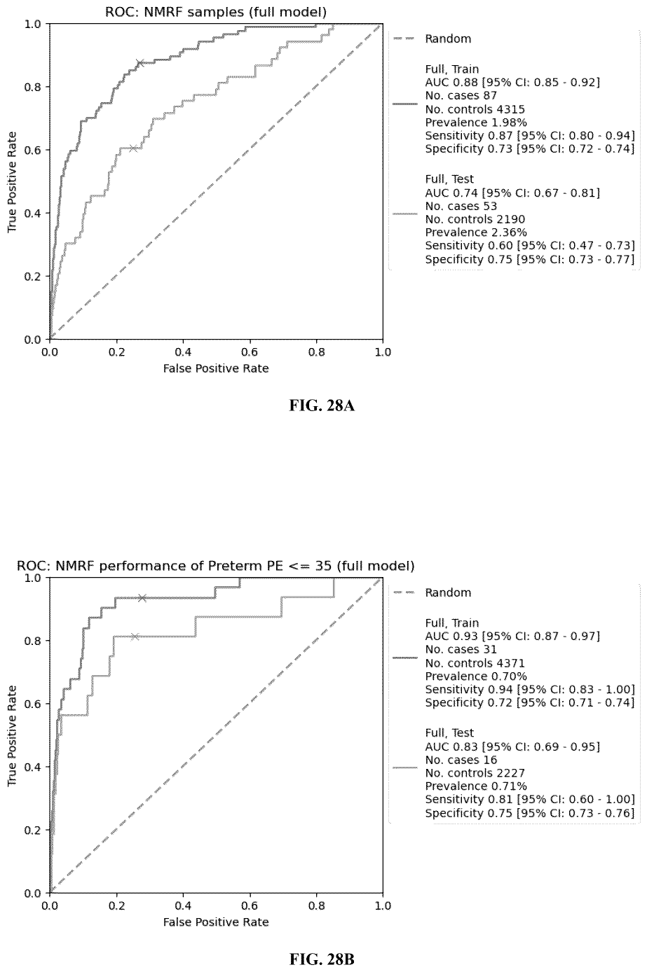

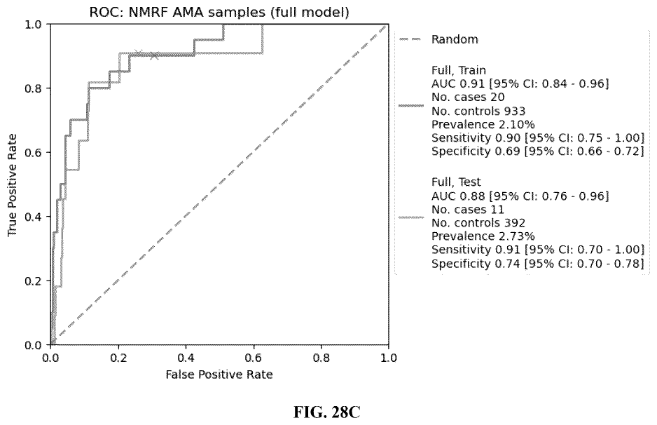

The classifier achieved a training area under the curve (AUC) of 0.88 and validated performance AUC of 0.74 (95% CI 0.67-0.81) with sensitivity=0.60 and specificity=0.75 (N=53 cases and N=2,190 controls) for predicting preterm preeclampsia (PAPPA2+ preeclampsia) in the population without any major risk factors, as shown in FIG. 28A.

The classifier achieved a training area under the curve (AUC) of 0.93 and validated performance AUC of 0.83 (95% CI 0.69-0.95) with sensitivity=0.81 and specificity=0.75 (N=16 cases and N=2,227 controls) for predicting preterm preeclampsia (PAPPA2+ preeclampsia) with delivery less than or equal to 35 weeks of gestation in the population without any major risk factors, as shown in FIG. 28B.

The classifier achieved a training area under the curve (AUC) of 0.91 and validated performance AUC of 0.88 (95% CI 0.76-0.96) with sensitivity=0.91 and specificity=0.74 (N=11 cases and N=392 controls) for predicting preterm preeclampsia (PAPPA2+ preeclampsia) in the population without any major risk factors and maternal age over 35 years, as shown in FIG. 28C.

### Example 19: Molecular Phenotypes of Hypertensive Disorders of Pregnancy

Hypertensive disorders of pregnancy (HDP), such as preeclampsia (PE), may be key drivers of maternal morbidity and mortality. Currently, HDP may encompass diverse clinical phenotypes that are assumed to share similar biological determinants and thus, clinical strategies lack precision-based approaches to care. Using systems and methods of the present disclosure, RNA profiles from maternal blood were used to determine different molecular pathways that drive different phenotypes of HDP.

A geographically, ethnically, and racially diverse prospective cohort of 10,718 singleton pregnancies were collected. To train and validate biological signatures of HDP, which affected 23% of study participants, full transcriptome analysis was performed. From differential gene expression analyses, it was shown that overexpression of the placental gene PAPPA2 (p_{adj}=2×10⁻²²) was correlated with earlier delivery for PE in a dose-dependent manner (p=1.45×10⁻⁷). In subjects with HDP, who were not in the top quartile for PAPPA2 expression, the molecular signal for HDP was instead dominated by immune genes, such as CD163 (p_{adj,HDP}=10⁻¹⁴). Within cases, PAPPA2 was overexpressed in individuals without major risk factors (p=8.41×10⁻⁷), such as hypertension or history of PE. A PAPPA2-driven classifier detects cases without a known risk factor most accurately, In the validation dataset for this group, the classifier predicted PE diagnosed <37 weeks leading to preterm birth and/or severe features (n=2,243, AUC 0.74); for a subgroup with early preterm PE<=35 week delivery (n=2,243, AUC 0.83); and for a subgroup with advanced maternal age (n=403, AUC 0.88). The results revealed dose-dependent molecular drivers of the most severe forms of HDP in those without prior identifiable risk factors, thereby creating improved methods to apply precision-based diagnostics, treatment, monitoring, and clinical assessment to maternal health.

HDPs are on the rise in most parts of the world, and is a leading cause of morbidity and mortality among pregnant people. The etiology is poorly understood, with hypotheses such as viral causes, through immune reaction, or placental dysfunction. HDP may be a multi-etiology disorder, for which molecular underpinnings were investigated through full transcriptomic analysis from a large prospective cohort.

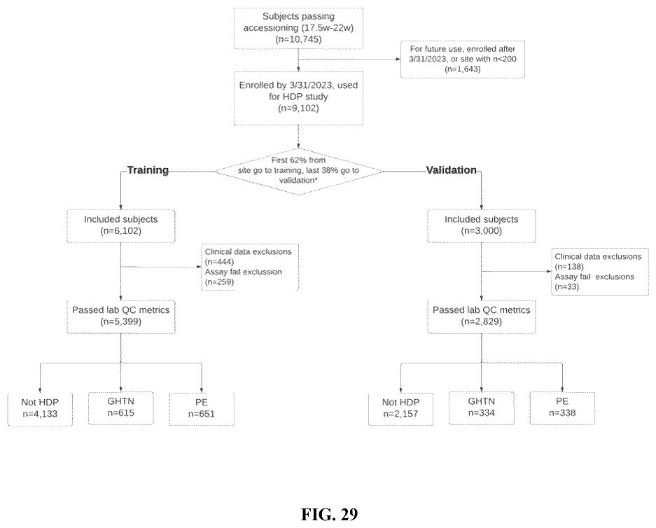

To investigate the underlying biology that drives different pregnancy complications, a prospective collection was performed of 10,745 blood samples from pregnant individuals between gestational weeks of 17.5 and 22. For the HDP studies presented in this example, a set of 9,102 samples were included. For sites where source data verification was not possible, all samples were included in the training data set. For all other sites, samples were split such that the first 62% collected were in the training set and the last 38% collected were in the validation data set (FIG. 29). All data was collected following best clinical practices, and all case labels of preeclampsia and any sample with unusual findings were adjudicated by an independent panel of maternal fetal medicine experts.

FIG. 29 shows a CONSORT diagram for studies in which molecular data is analyzed for association with clinical phenotypes, such that pregnancy-related disease states can be defined and assessed at a molecular level.

HDP is split into gestational hypertension and preeclampsia when following clinical guidelines. To explore how gene expression aligns with this split, the differentially expressed genes between preeclampsia and all other samples were investigated. PAPPA2 was revealed as the gene with lowest p-value and largest effect size. This result aligns well with studies of single-cell gene expression in early onset preeclampsia. Single-cell expression analysis revealed a differential expression of PAPPA2 in the nucleus of extravillous trophoblast and syncytiotrophoblast. Generally, PAPPA2 is expressed most highly in placenta and at a lower level in kidney; in this data, PAPPA2 expression correlated most with other genes expressed highly in placental cells, indicating placental origin.

Studies on cfDNA indicate that samples have large variation in fetal fraction measured in a blood sample. As the PAPPA2 signal from cfRNA is derived from placenta, a search was conducted for genes that may serve as corrections for variation in placental fraction. The search was started in samples from pregnant individuals carrying male fetuses, and identified three genes, VGLL3, SVEP1 and KRT7, that correlated strongly with total count from genes expressed from the Y chromosome. When applying a correction by regressing out signal from these genes across all placental genes, an increase in effect size was observed for PAPPA2 in the differential gene expression analyses.

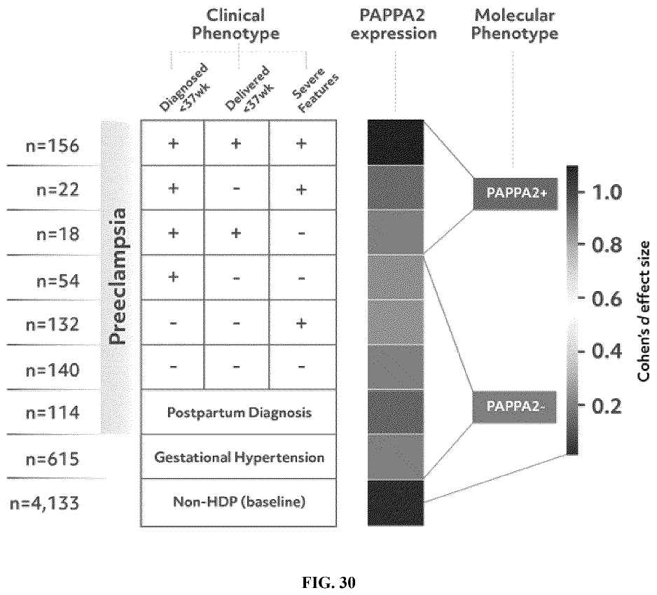

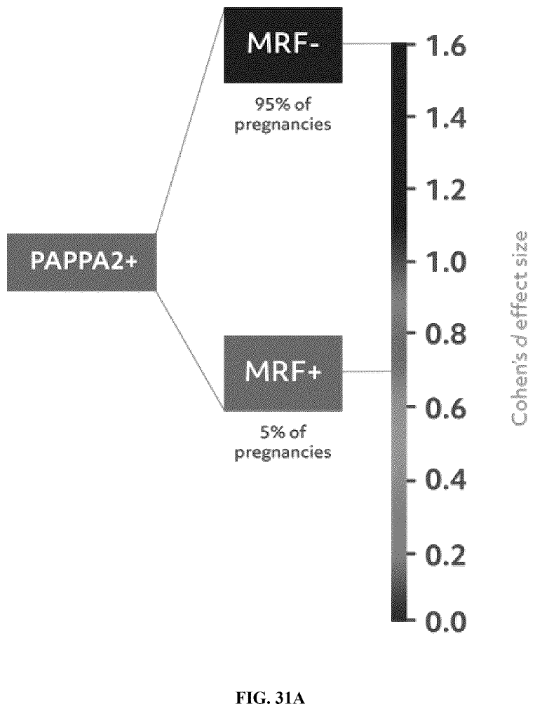

Clinically preeclampsia is often bucketed by time of diagnosis, time of delivery, and if preeclampsia presents with severe features. Preeclampsia samples were grouped by these labels and the effect size of PAPPA2 was overlaid for each of these groups (FIG. 30). Three subgroups emerged as having a large effect size for PAPPA2 (Cohen's d>0.8), with the remaining 5 subgroups all indicating a small to medium effect size. With this mapping of molecular signal to clinical phenotypes, two molecular distinct forms of preeclampsia were defined: 1) PAPPA2+ indicated by samples diagnosed before 37 weeks of gestation and having a preterm delivery and/or develop severe features, and 2) PAPPA2− preeclampsia which covers most forms of term preeclampsia, preeclampsia with postpartum diagnosis and gestational hypertension.

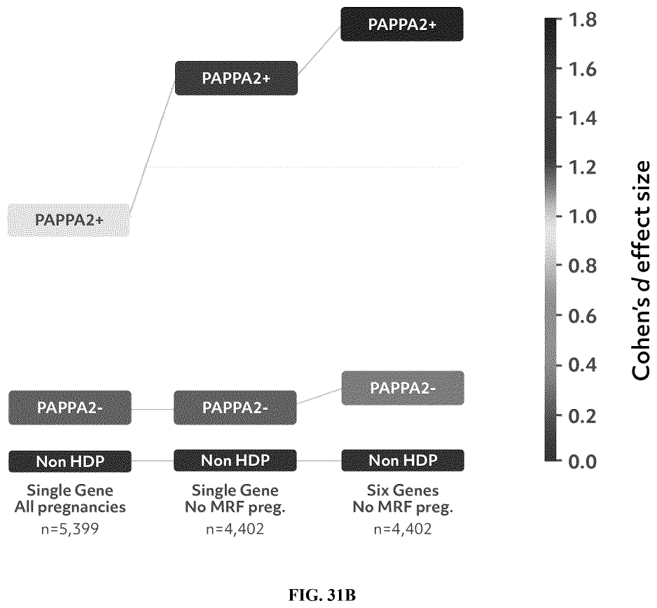

Several clinical risk factors for preeclampsia exist with some considered major risk factors: chronic hypertension, preeclampsia in previous pregnancy, diabetes mellitus, chronic kidney disease, systemic lupus erythematosus (SLE), Antiphospholipid syndrome (APS), and twin pregnancies (excluded in this study) (ACOG guideline). When the data was grouped by presence of a major risk factor, the effect size of PAPPA2+ samples diverged, where samples that have a major risk factor (MRF+) have lower effect size and samples no major risk factors (MRF−) have a very large effect size for PAPPA2 (FIG. 31A). Samples in the MRF− group also represent the group with the biggest unmet clinical need, as samples in MRF+ are all recommended for aspirin and closer monitoring.

Tracking the Cohen's d effect size across different groups shows a clear progression of signal, starting from all samples using a single gene, PAPPA2, to separate the groups (as defined in [0481]) into PAPPA2+, PAPPA2− and non HDP, when looking only at the samples with no major risk factors a much stronger effect is seen in the PAPPA2+ group, and finally when adding more genes to the classifier to use 6 predictive genes (PAPPA2, CD163, VSIG4, ADAM12, XAGE2, KISS1) the biggest effect size is achieved (FIG. 31B). For the latter group there is also a clear elevation of the PAPPA2− group, indicating that the additional genes are helping separate not only PAPPA2+ but also PAPPA2− from the non HDP group. This latter finding was further validated in a set of unseen validation samples, as described in the CONSORT diagram (FIG. 29), and a significant separation by genes only was confirmed.

When analyzing PAPPA2 expression across MRF− cases and comparing PAPPA2+ cases to non-PAPPA2+ cases, a dose-dependence was observed with earlier delivery in PAPPA2+ samples, but not in other samples, such that higher PAPPA2 expression is correlated with earlier delivery (FIGS. 25A-25B). Other causes of early delivery such as spontaneous preterm birth have no elevation in PAPPA2 signal.

Focusing on the PAPPA2− population, a differential gene expression analysis revealed a signature dominated by immune genes, which are shared between PAPPA2− preeclampsia and gestational hypertension, indicating that these subgroups of HDP are molecularly more similar (as can be shown by QQ plots for the two groups).

Further, SLCO2B1 was observed to be differentially expressed by preeclampsia status. In control cases, this gene was observed to be significantly differentially expressed by aspirin prescription, with higher expression in individuals who were prescribed aspirin. As SLCO2B1 is involved in cellular aspirin transport, these results may indicate a mechanistic reasons for the beneficial effect of aspirin on treating preeclampsia.

When considering other placental disorders such as the lowest three percentiles of birth weights, these samples are not driven by PAPPA2 but instead COL24A1, a collagen gene family member expressed in the cervix and involved in regulation of fibrillogenesis enduring fetal development.

To design and construct a classifier for samples in the PAPPA2+ group, a search was conducted for genes that can provide an orthogonal signal to that of PAPPA2. As shown by the Q-Q plots in FIGS. 23A and 23C, the search was conducted across the set of all PAPPA2+ samples (FIG. 23A) and the subset of samples that are MRF− (FIG. 23C).

Genes for inclusion in the classifier were selected based on cross validation splits in the training data, with the final list observed in at least a pre-determined percentage of splits. Genes selected for inclusion in the classifier included PAPPA2, CD163, VSIG4, KISS1, XAGE2, and ADAM12.

To optimize predictive accuracy of the classifier, additional clinical metrics were investigated that may further boost classifier performance. When adding clinical metrics to the search, clinical metrics including mean arterial blood pressure (e.g., measured just prior to blood draw) and body mass index (BMI) were observed to increase predictive accuracy. BMI may be anti-correlated with fetal fraction in cfDNA studies, adding more support to the correction genes for fetal fraction estimation.

In training, it was observed that the correction genes, in particular VGLL3, introduce a sex bias in the classification, giving females a slightly higher sensitivity and lower specificity. To balance performance between the fetal sexes, a fetal sex predictor was constructed, and the probability output of that model was used as an input in the classifier for PAPPA2+ preeclampsia, thereby fully removing sex bias in the classifier.

To validate the classifier, performance was assessed across the MRF− subset of the 3,000 reserved validation cases. Here, an AUC of 0.74 was observed, with a sensitivity of 60% and a specificity of 75% (FIG. 28A).

Further, with the dose-dependent effect observed for PAPPA2, cases with deliveries less than or equal to 35 0/7 weeks were also analyzed, for which an AUC of 0.83 was observed with a sensitivity of 81% and a specificity of 75% (FIG. 28B).

Further, classifier performance was assessed across the moderate risk factor groups defined in ACOG guidelines (“Preeclampsia and Pregnancy”, American College of Obstetricians and Gynecologists, December 2021, accessed at www.acog.org/-/media/project/acog/acogorg/womens-health/files/infographics/preeclampsia-and-pregnancy.pdf, which is incorporated by reference herein in its entirety). Here, similar classifier performance was observed for various risk factor group characteristics (including nulliparous status, black race, and obesity); further, an AUC of 0.88 was observed in the advanced maternal age group, with a sensitivity of 91% and specificity of 74% (FIG. 28C).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations, or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-116. (canceled)

117. A method for providing a clinical intervention to a pregnant subject, comprising:
(a) obtaining a cell-free biological sample from the pregnant subject;
(b) assaying the cell-free biological sample to determine an expression level of at least one pregnancy-related hypertensive disorder associated gene, wherein the at least one pregnancy-related hypertensive disorder-associated gene is differentially expressed in a first population of subjects with pregnancy-related hypertensive disorder as compared to a second population of subjects without the pregnancy-related hypertensive disorder;
(c) computer processing the expression level of the at least one pregnancy-related hypertensive disorder-associated gene determined in (b) (i) with a trained machine learning algorithm or (ii) against a reference value;
(d) determining, based at least in part on the computer processing in (c), that the pregnant subject has an elevated risk of having the pregnancy-related hypertensive disorder; and
(e) providing the clinical intervention to the pregnant subject to reduce the elevated risk of having the pregnancy-related hypertensive disorder, responsive to the determining in (d).

118. The method of claim 117, wherein the cell-free biological sample comprises cell-free ribonucleic acid (cfRNA), cell-free deoxyribonucleic acid (cfDNA), or protein.

119. The method of claim 117, wherein the cell-free biological sample is obtained from plasma, serum, urine, saliva, amniotic fluid, or derivatives thereof.

120. The method of claim 117, wherein the pregnant subject is asymptomatic for the pregnancy-related hypertensive disorder.

121. The method of claim 117, wherein the pregnancy-related hypertensive disorder comprises a molecular subtype of pregnancy-related hypertensive disorder.

122. The method of claim 121, wherein the molecular subtype of pregnancy-related hypertensive disorder is selected from the group consisting of chronic or pre-existing hypertension, superimposed preeclampsia, gestational hypertension, preeclampsia with severe features, preeclampsia without severe features, eclampsia, and HELLP syndrome.

123. The method of claim 121, wherein the molecular subtype of pregnancy-related hypertensive disorder is selected from the group consisting of history of chronic or pre-existing hypertension, superimposed pregnancy-related hypertensive disorder, presence or history of gestational hypertension, presence or history of pregnancy-related hypertensive disorder without severe features, presence or history of pregnancy-related hypertensive disorder with severe features, presence or history of eclampsia, and presence or history of HELLP syndrome.

124. The method of claim 117, wherein the at least one pregnancy-related hypertensive disorder-associated gene is selected from the group consisting of genes listed in Table 5, genes listed in Table 6, genes listed in Table 7, genes listed in Table 8, genes listed in Table 21, genes listed in Table 22, genes listed in Table 23, genes listed in Table 24, genes listed in Table 25, genes listed in Table 26, and genes listed in Table 27.

125. The method of claim 117, wherein computer processing the expression level of the at least one pregnancy-related hypertensive disorder-associated gene determined in (b) is with the trained machine learning algorithm.

126. The method of claim 117, wherein computer processing the expression level of the at least one pregnancy-related hypertensive disorder-associated gene determined in (b) is against the reference value.

127. The method of claim 117, wherein the clinical intervention comprises administering a therapeutic to the pregnant subject.

128. The method of claim 127, wherein the therapeutic comprises metformin, statins, proton pump inhibitors, sulfasalazine, eculizumab, or molecular-target strategies, or any combination thereof.

129. The method of claim 117, wherein the clinical intervention comprises use of aspirin, vitamin D, or calcium supplementations.

130. The method of claim 117, wherein the assaying comprises sequencing, mass spectrometry, chromatography, high performance liquid chromatography, ion mobility spectrometry, immunoassay, polymerase chain reaction (PCR), or a combination thereof.

131. A method for detecting a presence or an elevated risk of fetal growth restriction (FGR) of a fetus of a pregnant subject, comprising:
(a) obtaining a cell-free biological sample from the pregnant subject;
(b) assaying the cell-free biological sample to determine an expression level of at least one FGR-associated gene, wherein the at least one FGR-associated gene is differentially expressed in a first population of maternal subjects with a fetus having FGR as compared to a second population of maternal subjects with a fetus not having FGR;
(c) processing the expression level of the at least one FGR-associated gene determined in (b) (i) with a trained machine learning algorithm or (ii) against a reference value;
(d) determining, based at least in part on the processing in (c), that the fetus of the pregnant subject has the presence or the elevated risk of the fetal growth restriction; and
(e) administering a treatment to the pregnant subject for the presence or the elevated risk of the fetal growth restriction, responsive to the determining in (d).

132. The method of claim 131, wherein the cell-free biological sample comprises cell-free ribonucleic acid (cfRNA), cell-free deoxyribonucleic acid (cfDNA), or protein.

133. The method of claim 131, wherein the cell-free biological sample is obtained from plasma, serum, urine, saliva, amniotic fluid, or derivatives thereof.

134. The method of claim 131, wherein the fetal growth restriction is associated with baby birth weight at birth percentiles less than or equal to 10th, 9th, 8th, 7th, 6th, 5th, 4th, 3rd, 2nd, or 1st percentile in a reference population of baby birth weights.

135. The method of claim 131, wherein the FGR comprises a molecular subtype of FGR, wherein the molecular subtype of FGR comprises a subtype of intrauterine growth resulting in a baby that is born small for gestational age (SGA).

136. The method of claim 131, wherein the at least one FGR-associated gene is selected from the group consisting of RHBDD1, PTK2, SIRPA, HCK, GCA, NLRC4, CEBPD, ZC3H12C, B4GALT5, ST3GAL3, ZFPM2, PTP4A2P2, PLCB4, COL24A1, IMPDH1, RAB3D, SLA, GRAP2, LRRC4, RGS6, ALOX5, NCKAP1, GTF2IP4, PDGFRA, PFKFB4, LPCAT2, RASGRP4, HSPA1A, PRAM1, ARHGAP6, ASAP2, CDC14B, RP2, genes listed in Table 19, and genes listed in Table 20.

137. The method of claim 131, wherein the assaying comprises sequencing, mass spectrometry, chromatography, high performance liquid chromatography, ion mobility spectrometry, immunoassay, polymerase chain reaction (PCR), or a combination thereof.
